(19) United States
(12) Patent Application Publication
(10) Pub. No.: US 2008/0213377 A1
(43) Pub. Date: Sep. 4, 2008

(54) Delivery of Nanoparticles and/or Agents to Cells

(76) Inventors: Sangeeta N. Bhatia, Lexington, MA (US); Todd Harris, Winthrop, MA (US); Amit Agrawal, Medford, MA (US); Dal-Hee Min, Daejeon (KR); Austin M. Derfus, Solana Beach, CA (US); Geoffrey von Maltzahn, Boston, MA (US)

Correspondence Address: CHOATE, HALL & STEWART LLP, TWO INTERNATIONAL PLACE, BOSTON, MA, 02110, US

(21) Appl. No.: 11/952,614

(22) Filed: Dec. 7, 2007

Related U.S. Application Data
(60) Provisional application No. 60/969,389, filed on Aug. 31, 2007.
(60) Provisional application No. 60/873,897, filed on Dec. 8, 2006.

(51) Int. Cl. A61K 9/14; C07K 2/00; C07H 21/00; C07C 59/00; C07H 1/00

(52) U.S. Cl. 424/489; 530/300; 536/22.1; 554/1; 536/1.11

(57) ABSTRACT

The present invention provides systems, methods, and compositions for targeted delivery of nanoparticles and/or agents to tissues, cells, and/or subcellular locales. In general, compositions comprise a nanoparticle (e.g. quantum dot, polymeric particle, etc.), at least one modulating entity (such as a targeting moiety, transfection reagent, protective entity, etc.), and at least one agent to be delivered (e.g. therapeutic, prophylactic, and/or diagnostic agent). The present invention provides methods of making and using nanoparticle entities in accordance with the present invention.

### RELATED APPLICATIONS

This application claims priority under 35 U.S.C. § 119(e) to U.S. Provisional Patent Applications 60/873,897, filed Dec. 8, 2006 (“the '897 application”), and 60/969,389, filed Aug. 31, 2007 (“the '389 application”). The entire contents of the '897 application and the '389 application are incorporated herein by reference in their entirety.

### GOVERNMENTAL SUPPORT

The United States Government has provided grant support utilized in the development of the present invention. In particular, National Institutes of Health (contract numbers N01-C0-37117, R01-CA-124427-01, U54 CA119349, U54 CA119335, and EB 006324) have supported development of this invention. The United States Government has certain rights in the invention.

### BACKGROUND OF THE INVENTION

Considerable attention has been devoted to developing reagents and methods for delivering agents to particular tissues, cells, and/or subcellular locations. To give but one example, significant efforts have centered on the delivery of relatively large DNA constructs containing a gene of interest into the nucleus of eukaryotic cells in order to achieve either stable or transient increases in expression of the gene. More recently, with the discovery of RNA interference (RNAi), there has been increased interest in reagents and methods for delivering RNA to cells.

RNAi is a gene silencing mechanism triggered by double-stranded RNA (dsRNA) that has emerged as a powerful tool for studying gene function. Since the discovery of RNAi (Fire et al., Nature, 391:806; incorporated herein by reference), the evolutionarily conserved process has been exploited to analyze the functions of nearly every gene in model organisms C. elegans (Kamath et al., 2003, Nature, 421:231; and Maeda et al., 2001, Curr. Biol., 11: 171; Boutros et al., 2004, Science, 303:832; all of which are incorporated herein by reference) and a host of mammalian genes including approximately 23% of the sequenced human genes (Zheng et al., 2004, Proc. Natl. Acad. Sci., USA, 101: 135; and Novina and Sharp, 2004, Nature, 430:161; both of which are incorporated herein by reference). RNAi has also been used to effectively inhibit expression of viral genes in mammalian cells, resulting in inhibition of viral infection (Ge et al., 2004, Proc. Natl. Acad. Sci., USA, 101:8676; Radhakrishnan et al., 2004, Virology, 323:173; and Hu et al., 2004, Virus Res., 102:59; all of which are incorporated herein by reference). In addition to viral target genes, RNAi has been used to silence expression of a wide range of endogenous disease-related genes in mammalian cells, suggesting a variety of potential therapeutic applications (see, e.g. Dykxhhorn et al., 2003, Nat. Rev. Mol. Cell. Biol., 4:457; incorporated herein by reference).

RNAi is frequently achieved in mammalian cell culture or in vivo by the administration of short dsRNA duplexes, typically with symmetric 2-3 nucleotide 3′ overhangs, referred to as siRNA. If the RNAi effector sequence is potent and the siRNA delivered efficiently throughout the cell culture, remarkably specific post-transcriptional inhibition of gene expression can be achieved (Chi et al., 2003, Proc. Natl. Acad. Sci., USA, 100:6343; and Semizarov et al., 2003, Proc. Natl. Acad. Sci., USA, 100:6347; both of which are incorporated herein by reference). However, inefficient and heterogeneous delivery of siRNA is frequently observed in cell cultures, causing variable levels of gene silencing and potentially confounding the interpretation of genotype/phenotype correlations (Raab and Stephanopoulos, 2004, Biotechnol. Bioeng., 88:121; Huppi et al., 2005, Mol. Cell, 17: 1; Spagnou et al., 2004, Biochemistry, 43:13348; and Oberdoerffer et al., 2005, Mol. Cell, 25:3896; all of which are incorporated herein by reference). Without the means to address and resolve transfection variability, the utility of RNAi in eukaryotes will only be fully realized in cell types that have been thoroughly optimized for siRNA delivery (McManus and Sharp, 2002, Nat. Rev. Genet., 3:737; incorporated herein by reference).

The importance of high transfection efficiency has been spotlighted by numerous reports investigating methods to either improve RNAi delivery (Muratovska and Eccles, 2004, FEBS Lett., 558:63; Lorenz et al., 2004, Bioorg. Med. Chem. Lett., 14:4975; Schiffelers et al., 2004, Nuc. Acid. Res., 32:e149; and Itaka et al, 2004, J. Am. Chem. Soc., 126:13612; all of which are incorporated herein by reference) or screen for efficient knockdown. In the latter case, typical strategies involve monitoring fluorescently end-modified siRNAs (Manoharan, 2004, Curr. Opin. Chem. Biol., 8:570; and Chiu et al., 2004, Chem. Biol., 11:1165; both of which are incorporated herein by reference) or co-transfecting reporter plasmids and selecting for high transfection by fluorescence or antibiotic-resistance (Kumar et al., 2003, Genome Res., 13:2333; incorporated herein by reference). These techniques enable one-time selection of highly transfected cells yet discard moderately-silenced cells, which may be of interest to the study. For example, varying degrees of RNAi-mediated downregulation in the tumor suppressor gene Trp53 have been shown to modulate expression of distinct pathological phenotypes both in vitro and in vivo (Hemann et al., 2003, Nat. Genet., 33:396; incorporated herein by reference). Moreover, rapid photobleaching of organic fluorophores and the limited selection of available reporters currently prevent RNAi tracking from being feasible in either long-term or multiplexed studies. The dyes commonly used to label siRNAs lose over half the intensity of fluorescent signal in 5-10 seconds (Wu et al., 2003, Nat. Biotechnol., 21:41; and Dahan et al., 2003, Science, 302:442; both of which are incorporated herein by reference). Meanwhile, fluorescent reporter plasmids, although meant to be continuously expressed by the cells, can require as long as 2 hours after transcription for the functional protein to be observable (Tsien, 1998, Ann. Rev. Biochem., 67:509; incorporated herein by reference). In addition, due to the limited availability of fluorophores and reporter proteins that have non-overlapping emission spectra, current screening methods that rely on exogenous administration of siRNAs to cells are incapable of simultaneous monitoring of multiple siRNA molecules.

Development of more effective methods for delivery of siRNA in vivo would enhance and expand the therapeutic possibilities of this technology. However, it has thus far been difficult to study siRNA delivery in animal models of human disease such as mice and rats. This difficulty confounds attempts to evaluate new siRNA delivery vehicles or to compare the efficacy and/or side effects of different siRNA sequences in vivo.

Thus there is a specific need in the art for improved methods for delivering functional RNAs such as siRNA to eukaryotic cells. There is also a general need for improved methods and systems for achieving targeted delivery of agents.

### SUMMARY OF THE INVENTION

The present invention provides compositions and methods for delivery of nanoparticle entities to specific locations such as tissues, cells, and/or subcellular locales. In some embodiments, nanoparticle entities are optically or magnetically detectable nanoparticles.

In some embodiments, nanoparticle entities are associated with one or more entities that modulate nanoparticle delivery. A modulating entity may be physically associated with the nanoparticle. In some embodiments, a modulating entity and a nanoparticle are either covalently or non-covalently conjugated to one another.

In some embodiments, a modulating entity may be selected from the group consisting of targeting entities, transfection reagents, translocation entities, endosome escape entities, entities that alter activity of an agent, entities that mediate controlled release of an agent, etc. In specific embodiments, a modulating entity is a targeting entity which directs a nanoparticle to a specific tissue, cell, or subcellular locale.

The present invention provides compositions and methods for delivery of an agent to specific locations such as tissues, cells, and/or subcellular locales. In some embodiments, one or more agents to be delivered are associated with one or more nanoparticle entities. An agent to be delivered may be physically associated with a nanoparticle. In some embodiments, an agent to be delivered and a nanoparticle are either covalently or non-covalently conjugated to one another. In some embodiments, an agent to be delivered is releasably associated with a nanoparticle. In some such embodiments, a modulating entity alters release of the agent from the nanoparticle. A modulating entity may or may not remain associated with the nanoparticle.

Thus, the present invention provides compositions in which a modulating entity and/or an agent to be delivered is/are associated with a nanoparticle entity such that the modulating entity directs delivery of the nanoparticle entity and/or the agent to be delivered to the desired location.

In some embodiments, the agent to be delivered is a therapeutic, diagnostic, and/or prophylactic agent. Exemplary agents to be delivered in accordance with the present invention include, but are not limited to, small molecules and drugs, nucleic acids, proteins and peptides (including antibodies), lipids, carbohydrates, vaccines etc., and/or combinations thereof. In specific embodiments, the biologically active agent is or includes a functional RNA. Such a functional RNA may, for example, be selected from the group consisting of: siRNAs, shRNAs, tRNAs, and ribozymes.

In some embodiments, the invention provides cells comprising a modulating entity, an optically or magnetically detectable nanoparticle, and a functional RNA, wherein the functional RNA was not synthesized by the cell.

The invention provides methods of preparing a composition comprising the step of contacting an optically or magnetically detectable nanoparticle, an agent, and a modulating entity. The invention provides complexes comprising an optically or magnetically detectable nanoparticle, an agent, and a modulating entity. In some embodiments, the nanoparticle is a quantum dot and the agent is an RNAi agent (e.g. an siRNA or shRNA). In some embodiments, the modulating entity is a transfection reagent. In some embodiments, the modulating entity is a transfection reagent. In some embodiments, the modulating entity is a targeting entity. In some embodiments, the targeting entity is a peptide. In some embodiments, the modulating entity is polyethylene glycol. While not wishing to be bound by any theory, PEG may function as a modulating entity by improving circulation time of a nanoparticle and/or reducing degradation of an agent. In some embodiments, the modulating entity may mediate triggered release of an agent. Exemplary modulating entities that may mediate triggered release of an agent include, but are not limited to, transfection reagents, light, or heat.

In some embodiments, the invention provides methods of monitoring delivery of an agent to a cell comprising steps of: (a) contacting the cell with an optically or magnetically detectable nanoparticle and an agent; and (b) analyzing the cell to detect the presence, absence, or amount of the nanoparticle in the cell, wherein presence of the nanoparticle in the cell is indicative of presence of the agent in the cell. In some embodiments, the amount of the nanoparticle in the cell is indicative of the amount and/or activity of the agent in the cell. In certain embodiments, the agent is an RNAi agent (e.g. an siRNA or shRNA), and the nanoparticle is a quantum dot.

In some embodiments, the invention provides kits comprising at least one nanoparticle, at least one modulating entity, and at least one agent to be delivered. In certain embodiments, the agent is an RNAi agent and the nanoparticle is a quantum dot.

The invention provides compositions and methods such as those described above comprising a multiplicity of different agents and a multiplicity of optically or magnetically distinguishable nanoparticles, wherein each of a multiplicity of different agents is physically associated with a nanoparticle that is distinguishable from nanoparticles associated with other agents. The invention may be used to target the delivery of one agent or of multiple agents in vivo.

In various embodiments, the invention provides methods for the identification and/or selection of cells that have taken up siRNAs in an amount sufficient to silence one or more target genes, cells that have taken up approximately equal amounts of the same siRNA or of different siRNAs, cells that have taken up siRNAs in amounts that do not saturate the RNAi machinery, cells that have taken up siRNAs in amounts that do not result in non-sequence specific effects, cells that have taken up siRNAs in amounts that do not result in “off-target” silencing, etc.

This application refers to various patent publications, all of which are incorporated herein by reference. For purposes of the present invention, the chemical elements are identified in accordance with the Periodic Table of the Elements, CAS version, Handbook of Chemistry and Physics, 75th Ed., inside cover, and specific functional groups are generally defined as described therein.

### BRIEF DESCRIPTION OF THE DRAWING

FIG. 1: Quantum dot/siRNA complexes allow sorting of gene silencing in cell populations. (Panel A) Schematic representation of cells co-transfected with quantum dots (QDs) and siRNA and analyzed for intracellular fluorescence by flow cytometry. Histograms depict fluorescence distributions of control murine fibroblast cells, Lmna siRNA-treated cells, and Lmna siRNA/QD-treated cells. FACS was used to gate and sort the high 10% (H) fluorescence and low 10% (L) fluorescence of each distribution. L⁻ and H⁻ point to gates for the siRNA only histogram. L⁺ and H⁺ indicate gates for the siRNA/QD histogram. (Panel B) Representative Western blot of Lamin A/C protein expression levels in sorted cells with β-actin as loading control. Control lanes are protein from cells mock-transfected with liposome reagent only and sorted (L, H). The absence of QDs is indicated by a minus sign (−) and the presence of QDs is indicate by a plus sign (+). (Panel C) Band densitometry analysis of Western blots from replicate experiments. Error bars represent standard error of the mean (n=3). ***P<0.001 (one-way ANOVA).

FIG. 2: Immunofluorescence staining of Lamin A/C nuclear protein. (Panel A) Unsorted cells (U) transfected with Lmna siRNA alone display heterogenous staining for Lamin A/C nuclear protein (red) throughout the cell population. White arrows highlight examples of cells with weak lamin staining among cells stained strongly for lamin. (Panel B) Cells co-transfected with Lmna siRNA and green QDs exhibit bright lamin staining and lack of QDs in low-gated (L⁺) cell subpopulations and (Panel C) weak lamin staining and presence of QDs in high-gated (H⁺) cell subpopulations (shown enlarged in inset). Scale bars 75 μm.

FIG. 3: Optimization of QD concentration for siRNA tracking. Lmna siRNA (100 nM) and 1 μg, 2.5 μg, 5 μg, or 10 μg QD were co-transfected into murine fibroblasts and the cells FACS-sorted for the low 10% (L⁺) and high 10% (H⁺) of intracellular fluorescence distribution. (Panel A) Protein expression of sorted cells assayed by Western blot, β-actin loading control. Unsorted, lipofectamine only control (U) represented 100% lamin A/C protein expression. (Panel B) Western blot band densitometry analysis of L⁺ and H⁺ bands shows an optimum QD concentration for obtaining high-efficiency silencing. (Panel C) Band density difference (L⁺ minus H⁺) reveals an optimum QD concentration for sorting most efficiently silenced from least efficiently silenced subpopulations.

FIG. 4: Sorting the effects of double gene knockdowns using two colors of QDs. (Panel A) Schematic representation of cells transfected simultaneously with Lmna siRNA/green QD complexes and T-cad siRNA/orange QD complexes. The low 8% (L⁺⁺, where ++ designates the presence of two colors of QDs) and high 8% (H⁺⁺) of the dual fluorescence dot plot was gated and isolated using FACS. (Panel B) Representative Western blot and (Panel C) corresponding band densitometry analysis of lamin A/C and T-cadherin protein levels in control unsorted (U) cells, unsorted (U) T-cad siRNA-treated cells, sorted T-cad/QD-treated cells (L⁺, H⁺), and sorted dual siRNA/dual QD-treated (L⁺⁺, H⁺⁺) cells.

FIG. 5: Fluorescence/phase micrographs of two color QD transfections. (Panel A) Low-gated cells (L⁺⁺, where ++ indicates the presence of two colors of QD) nearly lack orange or green QDs. (Panel B) High-gated cells (H⁺⁺) fluoresce brightly with punctate green and orange QDs (enlarged in inset). Scale bars 100 μm.

FIG. 6: Significant downstream gene knockdown effects of T-cadherin gene silencing are observed only in a homogenously silenced cell population. Murine 3T3 fibroblasts transfected with T-cad siRNA alone or with T-cad siRNA/QD complexes were FACS-sorted for low 10% (L) or high 10% (H) intracellular fluorescence. Symbols − and + indicate the absence or presence of QD during transfection. To study the stabilizing effect of non-parenchymal cell (3T3 fibroblast) protein expression on liver-specific function, control or transfected/sorted 3T3 cells were added to hepatocyte cultures 24 hours after hepatocyte seeding. Liver-specific function was assayed by measuring albumin synthesis and cytochrome P450 1A1 (CYP1A1) activity of cultured media sampled at 72 and 96 hours after 3T3 seeding and averaged. Error bars represent standard error of the mean (n=3). *P<0.05, **P<0.01, ***P<0.001 (one-way ANOVA statistical analysis test).

FIG. 7: Knockdown efficacy is not improved by transfecting higher doses of siRNA. 3T3 murine fibroblasts were transfected with 100 nM, 200 nM, 300 nM, or 400 nM Lmna siRNA and harvested for protein after 72 hours. (Panel A) Representative Western blot of Lamin A/C protein levels, β-actin loading control. (Panel B) Band densitometry analysis from replicate experiments, where error bars represent standard error of the mean (n=2).

FIG. 8: QD-labeled and fluorescein-labeled siRNA fluorescence in 3T3 murine fibroblasts. After continuous mercury lamp exposure, QD fluorescence is shown in Panel A and siRNA fluorescence is shown in Panel B. Scale bars are 25 μm.

FIG. 9: Silencing activity of QD/siRNA conjugates in mammalian cells. The upper portion of the figure shows reagents used to synthesize the conjugates. The lower left portion of the figure shows silencing activity of siRNA or QD/siRNA conjugates in HeLa cells. The lower right portion of the figure shows signal obtained from the internalized QD/siRNA conjugates.

FIG. 10: Schematic diagram illustrating multifunctional nanoparticles for siRNA delivery.

FIG. 11: Uptake of unconjugated QDs or QDs conjugated with a variety of different moieties. A fluorescence histogram shows uptake by HeLa cells of unconjugated QDs or QDs conjugated with a variety of different moieties.

FIG. 12. Attachment of F3 peptide leads to QD internalization in HeLa cells. Thiolated peptides (F3 and KAREC control) and siRNA were conjugated to PEG-amino QD705 particles using sulfo-SMCC. Particles were filtered to remove excess peptide or siRNA, and incubated with HeLa cell monolayers for 4 hours. Flow cytometry indicated the F3 peptide is required for cell entry (Panel A). The addition of free F3 peptide inhibits F3-QD uptake, while KAREC peptide does not, suggesting the F3 peptide and F3-labeled particles target the same receptor (Panel B). In Panel C, the relationship between number of F3 peptides per QD and cell uptake was examined. In these experiments, FITC-labeled peptide was conjugated to QDs using sulfo-LC-SPDP. For each formulation (black circles), peptide:QD ratio was determined by measuring the QD concentration by absorbance, then treating the conjugate with 2-mercaptoethanol, filtering out the QDs, and measuring the FITC fluorescence. Cell uptake increases dramatically with peptide number, but appears to saturate around 10-15 F3s per QD.

FIG. 13. Conjugation of siRNA to QDs with cleavable or non-cleavable cross-linkers. Thiol-modified siRNA was attached to PEG-amino QDs using the water-soluble heterobifunctional cross-linkers sulfo-SMCC and sulfo-LC-SPDP (Panel A). The cross-link produced by SPDP is cleavable with 2-mercaptoethanol (2-ME), while the bond attained with SMCC is covalent. Gel electrophoresis of the disulfide-linked conjugates indicated that no siRNA are electrostatically bound to the conjugate (Panel B). Upon treatment with 2-ME, the QD/siRNA cross-link is reduced and the siRNA migrated down the gel alongside siRNA standards (Panel C). QD/siRNA conjugates (or siRNA alone) were delivered to EGFP-expressing HeLa cells using Lipofectamine 2000 (cationic liposome reagent). Cells were trypsinized and assayed by flow cytometry 48 hours later. Comparison with control cells (treated with Lipofectamine alone) indicated the disulfide bond leads to superior EGFP knockdown (% reduction in geometric mean fluorescence) (Panel D). Comparing a dot-plot of cells treated with Lipofectamine alone (Panel E) or disulfide-linked QD/siRNA (Panel F) revealed a negative correlation between QD uptake and EGFP signal. Thus, the QD label can serve as a means of quantifying siRNA delivery and thus knockdown.

FIG. 14. Co-attachment of F3 peptide and siRNA cargo allows targeted EGFP knockdown upon delivery and endosome escape. Due to a limited number of attachment sites on the QDs, the goal of co-attachment was to maximize siRNA loading while conjugating sufficient F3 peptides to allow internalization (>15). Varying the F3:siRNA ratio resulted in a number of formulations (black circles, Panel A), with superior QDs observed using a reaction ratio of 4:1 and resulting in approximately 20 F3 peptide and approximately 1 siRNA per QD. EGFP-expressing HeLa cells were treated with 50 nM F3/siRNA-QDs for four hours and then washed with cell media. When assayed for green fluorescence 48 hours later, no knockdown was observed (“control,” Panel B). When these cells were treated with cationic liposomes (Lipofectamine 2000) immediately after removing the QDs and washing, an approximately 29% reduction in EGFP was observed. A lower concentration of QDs (10 nM) is less effective (21% knockdown). Incubation with KAREC-labeled particles followed by cationic liposomes leads to minimal particle internalization, and thus no knockdown. Fluorescence imaging of cells incubated with F3/siRNA QDs showed a reduced green fluorescence (Panel D), compared with control cells incubated with Lipofectamine alone (Panel C).

FIG. 15. Photoactivation of endosomal escape. Photosensitizers can effectively induce endosomal escape when combined with targeting peptide. A targeting peptide (cycCARSKNKDC; SEQ ID NO: 1), which binds to heparan sulfate proteoglycans, is conjugated to fluorecein, a photosensitizer, and incubated with glioblastoma cells (Panel A). After light irradiation for three minutes, fluorescence of the peptide was more evenly distributed, which indicates endosomal escape of the targeting peptide (Panel B).

FIG. 16. siRNA and targeting peptide are conjugated to nanoparticles via protease-cleavable peptide. Proteases such as matrix metalloproteases (MMPs) are upregulated in many types of tumors. Therefore, agents that are associated with nanoparticles via protease-cleavable bonds (red linker) are released from nanoparticles when nanoparticles reach tumor sites in vivo. Upon release, siRNAs can be internalized into cells.

FIG. 17. Multifunctional nanoparticles are multivalent, can be remotely actuated, and imaged noninvasively in vivo. (Panel A) Superparamagnetic nanoparticles embedded in tissue transduce external electromagnetic energy to heat, thereby melting oligonucleotide duplexes that act as heat-labile tethers to model drugs. (Panel B) In vitro, nanoparticles hybridized to fluorescein-conjugated 18mer were embedded in hydrogel plugs. Repeated EMF pulses of 5 minutes resulted in corresponding release of fluorescein (left). Alteration of oligonucleotide duplex length shifts response of heat-labile tether enabling complex release profiles. Low power EMF exposure results in release of fluorescein-conjugated 12mer whereas higher power results in simultaneous melting of both 12mer and 24mer tethers (right). (Panel C) Multifunctional nanoparticles were embedded in tumor phantoms and implanted subcutaneously in mice. Tumor phantoms were visualized using magnetic resonance imaging (right). Application of EMF to implanted phantoms with 18mer tethers resulted in release of model drugs and penetration into surrounding tissue (+EMF, right) when compared to unexposed controls (−EMF, left, scale bar=100 μm).

FIG. 18. siRNA degradation by serum can be reduced by co-immobilization with polyethylene glycol (PEG). PEG can be utilized to protect siRNA from serum nucleases by providing steric hindrance. siRNAs are conjugated to gold nanoparticles with PEG (Panel C) or without PEG (Panel B). siRNA content was analyzed by gel electrophoresis after incubation with 50% serum at 37° C. at various timepoints. Relatively strong gel band intensity corresponding to siRNA was observed in case of PEG protected siRNA-gold nanoparticles (Panel C) even after 24 hr incubation, compared to non-PEGylated siRNA (Panel B) or naked siRNA (Panel A).

FIG. 19: Schematic depiction of removable polymer coatings that veil and unveil bioactive ligands on a nanoparticle surface. A hydrophilic polymer (wavy-gray) linked via MMP cleavable substrates (jagged-yellow) veils the activity of a cell-internalizing domain (jagged-blue) on the surface of a magnetofluorescent nanoparticle. Veiled particles have extended circulation times that enable their passive accumulation in tumors. Extravasated particles are activated by MMP-2 in the microenvironment to unveil internalizing domains, which associate with the cell membrane and shuttle nanoparticles into cells.

FIG. 20: Optimization and characterization of nanoparticle veiling, activation, and internalization. (A) A library of nanoparticles with removable polymer coatings and a varying density of internalization ligands was screened for relative uptake by HT-1080 cancer cells before (veiled, green) and after (unveiled, blue) MMP cleavage. A density of 6 cell internalizing peptides per particle demonstrated optimum veiling and internalization. Error bars are standard deviations from three separate experiments. (B) Cells incubated with veiled and unveiled nanoparticles for 5 hours are imaged by (left) a fluorescence scanner or (right) MRI demonstrating the dual contrast properties of the nanoparticles and the correlated fluorescent and magnetic domain uptake of unveiled particles. (C) MMP-mediated removal of polymer coatings relieves TAMRA-iron quenching interactions enabling remote monitoring of protease activation. (D) The K_{cat}/Kₘ for peptide-polymer NPs (red) and free peptide (blue) was determined to be 8.42 and 26.7 μM⁻¹ hr⁻¹ respectively by measuring the cleavage of the substrate by MMP-2 over time. Polymer veiling and immobilization of the cleavable peptide substrate reduces its associated MMP-2 K_{cat}/Kₘ within a practical range, 3.2 fold.

FIG. 21: Effects of removable polymer coatings on the blood-clearance and tumor accumulation of nanoparticles. (A) Nanoparticles bearing removable polymer coatings (veiled) have improved blood clearance times compared with particles that have had the coating removed by MMPs (unveiled). Error bars indicate standard deviation of two or more animals. (B) Fluorescence molecular tomography (FMT) of two representative animals shows intravenous injections of veiled nanoparticles yield greater accumulation in tumors after 48 hours as compared to unveiled controls. (C) Quantitative analysis of nanoparticle accumulation in the tumor at 48 hours by FMT demonstrates superior accumulation of veiled particles as compared to unveiled controls. Error bars represent standard deviation of three animals. (D) Representative histological sections confirm the increased accumulation of veiled nanoparticles versus unveiled controls after 48 hours; nanoparticles (green), blood vessels (red), nuclei (blue). Scale bar is 250 μm. (E) T2 map of tumor and muscle regions of interest (ROIs) after intravenous injection show enhanced contrast from veiled nanoparticles in the tumor versus normal tissue (muscle) at 24 hours post-injection.

FIG. 22: Removable polymer coatings veil nanoparticles in the blood, but are effectively released in tumors. (A) Monitoring the release of TAMRA-iron quenching interactions shows that particles with cleavable L-isomer peptides (L-AA) are activated by MMPs, while particles with uncleavable D-isomer peptides (D-AA) remain intact. (B) Blood circulation time of cleavable (L-AA) particles and uncleavable (D-AA) controls are closely matched and passive accumulation of cleavable and uncleavable nanoparticles in tumors by FMT (inset) are the same. Error bars indicate standard deviation of three animals. (C) Representative RGB merge of nanoparticles (green), removable polymer (red), and nuclei (blue) in tumor sections harvested 48 hours after injection shows decreased colocalization of particles and removable polymer with cleavable peptides, but not uncleavable controls. 2-D fluorescence intensity scatter plots (insert) show quantitative loss in colocalized pixels (yellow), demonstrating the removal of L-AA removable polymers from particles in the tumor. Scale bar is 250 μm.

FIG. 23: Trafficking of unveiled nanoparticles by epifluorescence microscopy. MMP-activated (unveiled) nanoparticles incubated over HT-1080 cells are imaged at 1 hour, 3 hours, and 5 hours. At 1 hour, particles can be seen lining the cell membrane. Over longer time points, particles appear in punctate intracellular organelles that traffic to the nucleus. Internalization of veiled particles is not visible (insert). Scale bar=75 μm.

FIG. 24: Unveiling of nanoparticles initiates cell-uptake in other cell lines. (A) MMP-activated (unveiled) nanoparticles internalize in brain (GLIO 1431), prostate (TRAMP), and breast (MDA-MB-435) cancer cell-culture models. Internalization of veiled nanoparticles is not visible (insert). Scale bar=50 μm. (B) Fold increase in mean internalization of unveiled over veiled nanoparticles after incubation for 5 hours as measured by flow cytometry. Error bars are standard deviations of three separate experiments.

FIG. 25: Recombinant MMP-2 (2.5 μg/ml) or collagenase (20 μg/ml) removes peptide-PEG and relieves TAMRA-iron quenching interactions enabling monitoring of protease activation. Incubation with the broad-spectrum inhibitor, Galardin (25 μM, Biomol), inhibits activation by both enzyme formulations.

FIG. 26: Scheme and preparation of DendriMaPs. (A) Scheme of DendriMaPs. DendriMaPs present amine-terminated dendrons derived from PAMAM dendrimer (generation 4, cystamine core, blue). Positive charges on the surface allow siRNA (yellow) adsorption onto the DendriMaPs. (B) Preparation of DendriMaPs. Aminated MIONs (Magnetic Iron Oxide Nanoparticles, purple) were prepared according to a previously published protocol followed by conjugation of heterobifunctional linker (SPDP) and reduced Dendron resulting in roughly 50-70 dendrons per particle (there are approximately 7 cores in each particle).

FIG. 27: Characterization of DendriMaPs. (A) Characterization of siRNA adsorbed DendriMaPs. Solutions of siRNAs (1 μM) were mixed with DendriMaPs at various concentrations and the mixed solutions were incubated for 10 minutes prior to running a gel. (B) Gel band intensities corresponding to free siRNAs from (A) were used to quantitate free siRNA concentrations in the solutions of DendriMaPs at various concentrations. More than 90% of 1 μM siRNAs were adsorbed onto DendriMaPs at the concentration of 0.1 μM or higher.

FIG. 28: EGFP knockdown by DendriMaPs. (A) EGFP knockdown in stably transfected HeLa cells. DendriMaPs and control group siRNA carriers were incubated with siRNAs for 8 minutes in serum free culture medium and the resulting mixture was placed over the cells for 4 hours. After 4 hours, media was changed to serum containing media. KD was assessed after 48 hours using flow cytometry. (B) Images corresponding to EGFP KD observed in HeLa-GFP cells with or without EGFP siRNA.

FIG. 29: EGFR knockdown in glioblastoma cells using DendriMaPs. (A) Protein quantitation was carried out using western blot analysis. Band intensities corresponding to EGFR were normalized by GAPDH band intensities. More than 80% reduction of EGFR expression was observed at optimal condition. (B) mRNA levels of EGFR and GAPDH were characterized by real time PCR. A 50% reduction of EGFR mRNA was observed after cells were treated with formulation containing 100 nM of siRNA and 100 nM of DendriMaP.

FIG. 30: DendriMaPs promote endosomal escape. HeLa cells were incubated with 0.24 mM Calcein for 1 hour in presence of various delivery agents (dendrimer, MIONs, and/or DendriMaP). Subsequently, cells were washed to remove excess dye and images were taken using 20× objective. (A) The extent to which Calcein is released from the endosomes inside cells in presence of 100 nM siRNA and different delivery agents. A diffuse cellular distribution of the dye implies endosomal disruption, which is absent in Calcein only and Calcein+MION samples. (B) Fraction of cells with endosomal escape was calculated by counting 100-150 cells for each formulation at 4 different siRNA concentrations. While free dendrimers were able to promote some endosomal escape when siRNA concentration is below 100 nM, DendriMaPs were much more efficient at concentrations up to 1 μM. (C) High magnification image of a cell that received Calcein using DendriMaPs. Diffuse cellular distribution and clear nuclear uptake highlight the endosomal release of Calcein. Concentration of dendrimers was approximately 30 μM and dendrimer concentration on the DendriMaPs was equivalent to 7 μM of dendrimers.

FIG. 31: Loading of siRNAs on DendriMaPs compared with that on dendrimers. (A) DendriMaPs carry several free primary amine groups which mediate the electrostatic attachment of negatively charged siRNA. Further, since not all of the primary amines may be accessible due to steric hindrance, DendriMaPs may be able to mediate the uptake of particles into the cells. Also, each DendriMaPs has much greater number of buffering amines (C) compared to individual dendrimers and hence may serve as more efficient endosome lysis agents. (B) Dendrimers may not only consume all of their primary amines for electrostatic binding with the siRNA but also possess fewer buffering amines per dendrimer. These factors are likely to reduce both the uptake and the extent of endosome lysis when dendrimers are used for siRNA delivery. One could promote endosome lysis by using excess of free dendrimer. However, at higher concentrations, dendrimers are fairly toxic which limits their application.

FIG. 32: Coating MIONs with dendrimers induces uptake into lungs. (A) 20 μg of magnetic iron oxide particles (“MIONs”) or DendriMaP (i.e. MION+dendrimer) was injected into the tail vein of a mouse. After blood levels of nanoparticles were stabilized, the animal was sacrificed and organs were removed. Uptake was assessed by imaging IR fluorescent dye coupled to the nanoparticles. (B) Relative uptake of nanoparticles in various organs. (C) % injected dose retained in various organs.

FIG. 33: EGFR/GAPDH ratio is linear over a broad range of protein concentration and can be used to assess the extent of protein expression levels successfully. Average GPDH/EGFR Ratio: 1.183300323 (SD=0.14375537).

FIG. 34: Particles are coated with DendriMaPs and cleavable PEG moieties. Particles are able to circulate freely, and when the PEG moieties are cleaved away, particles are able to accumulate in the target cell (e.g. tumor) where the PEG has been cleaved. The cationic dendrons interact with the cell and are endocytosed, upon which they lyse the endosome and deliver the siRNA to the cytosol.

FIG. 35: Coating Nanoparticles Can Help Stabilize Nanoparticles. C32 polymer degradation at physiological pH reduces transfection efficiency over time (top panel). The present invention provides methods and systems for improving nanoparticle stability. For example, electrostatic peptide-PEG coating can prolong the half-life of C32 polymer complexes and preserve transfection efficiency when activated at malignant sites (bottom panel). C32 Nanoparticles degrade hydrolytically at pH 7.4, destroying their ability to transfect DNA in MDA-MB-432 cells as measured by the % cell population of cells that get transfected with GFP by flow cytometry. Electrostatically adsorbed protease cleavable polymer coatings stabilize C32 nanoparticles for several hours in a polymer concentration-dependent manner. When a coating (e.g. L-AA coating) is removed by protease activity, transfection ability is restored. Uncleavable polymer coatings (e.g. D-AA) remain unable to transfect DNA into MDA-MB-432 cells after incubation with the protease.

### DEFINITIONS

Agent to be delivered: As used herein, the phrase “agent to be delivered” refers to any substance that can be delivered to a tissue, cell, or subcellular locale. In some embodiments, the agent to be delivered is a biologically active agent, i.e., it has activity in a biological system and/or organism. For instance, a substance that, when administered to an organism, has a biological effect on that organism, is considered to be biologically active.

Amino acid: As used herein, term “amino acid,” in its broadest sense, refers to any compound and/or substance that can be incorporated into a polypeptide chain. In some embodiments, an amino acid has the general structure H₂N—C(H)(R)—COOH. In some embodiments, an amino acid is a naturally-occurring amino acid. In some embodiments, an amino acid is a synthetic amino acid; in some embodiments, an amino acid is a D-amino acid; in some embodiments, an amino acid is an L-amino acid. “Standard amino acid” refers to any of the twenty standard L-amino acids commonly found in naturally occurring peptides. “Nonstandard amino acid” refers to any amino acid, other than the standard amino acids, regardless of whether it is prepared synthetically or obtained from a natural source. As used herein, “synthetic amino acid” encompasses chemically modified amino acids, including but not limited to salts, amino acid derivatives (such as amides), and/or substitutions. Amino acids, including carboxy- and/or amino-terminal amino acids in peptides, can be modified by methylation, amidation, acetylation, and/or substitution with other chemical groups that can change the peptide's circulating half-life without adversely affecting their activity. Amino acids may participate in a disulfide bond. The term “amino acid” is used interchangeably with “amino acid residue,” and may refer to a free amino acid and/or to an amino acid residue of a peptide. It will be apparent from the context in which the term is used whether it refers to a free amino acid or a residue of a peptide.

Animal: As used herein, the term “animal” refers to any member of the animal kingdom. In some embodiments, “animal” refers to humans, at any stage of development. In some embodiments, “animal” refers to non-human animals, at any stage of development. In certain embodiments, the non-human animal is a mammal (e.g., a rodent, a mouse, a rat, a rabbit, a monkey, a dog, a cat, a sheep, cattle, a primate, and/or a pig). In some embodiments, animals include, but are not limited to, mammals, birds, reptiles, amphibians, fish, insects, and/or worms. In some embodiments, an animal may be a transgenic animal, genetically-engineered animal, and/or a clone.

Antibody: As used herein, the term “antibody” refers to any immunoglobulin, whether natural or wholly or partially synthetically produced. All derivatives thereof which maintain specific binding ability are also included in the term. The term also covers any protein having a binding domain which is homologous or largely homologous to an immunoglobulin binding domain. Such proteins may be derived from natural sources, or partly or wholly synthetically produced. An antibody may be monoclonal or polyclonal. An antibody may be a member of any immunoglobulin class, including any of the human classes: IgG, IgM, IgA, IgD, and IgE. As used herein, the terms “antibody fragment” or “characteristic portion of an antibody” are used interchangeably and refer to any derivative of an antibody which is less than full-length. In general, an antibody fragment retains at least a significant portion of the full-length antibody's specific binding ability. Examples of antibody fragments include, but are not limited to, Fab, Fab′, F(ab′)2, scFv, Fv, dsFv diabody, and Fd fragments. An antibody fragment may be produced by any means. For example, an antibody fragment may be enzymatically or chemically produced by fragmentation of an intact antibody and/or it may be recombinantly produced from a gene encoding the partial antibody sequence. Alternatively or additionally, an antibody fragment may be wholly or partially synthetically produced. An antibody fragment may optionally comprise a single chain antibody fragment. Alternatively or additionally, an antibody fragment may comprise multiple chains which are linked together, for example, by disulfide linkages. An antibody fragment may optionally comprise a multimolecular complex. A functional antibody fragment typically comprises at least about 50 amino acids and more typically comprises at least about 200 amino acids.

Approximately: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Associated with: As used herein, the terms “associated with,” “conjugated,” “linked,” “attached,” and “tethered,” when used with respect to two or more moieties, means that the moieties are physically associated or connected with one another, either directly or via one or more additional moieties that serves as a linking agent, to form a structure that is sufficiently stable so that the moieties remain physically associated under the conditions in which structure is used, e.g., physiological conditions. In some embodiments, the moieties are attached to one another by one or more covalent bonds. In some embodiments, the moieties are attached to one another by a mechanism that involves specific (but non-covalent) binding (e.g. streptavidin/avidin interactions, antibody/antigen interactions, etc.). In some embodiments, a sufficient number of weaker interactions can provide sufficient stability for moieties to remain physically associated.

Biocompatible: As used herein, the term “biocompatible” refers to substances that are not toxic to cells. In some embodiments, a substance is considered to be “biocompatible” if its addition to cells in vivo does not induce inflammation and/or other adverse effects in vivo. In some embodiments, a substance is considered to be “biocompatible” if its addition to cells in vitro or in vivo results in less than or equal to about 50%, about 45%, about 40%, about 35%, about 30%, about 25%, about 20%, about 15%, about 10%, about 5%, or less than about 5% cell death.

Biodegradable: As used herein, the term “biodegradable” refers to substances that are degraded under physiological conditions. In some embodiments, a biodegradable substance is a substance that is broken down by cellular machinery. In some embodiments, a biodegradable substance is a substance that is broken down by chemical processes.

Biologically active: As used herein, the phrase “biologically active” refers to a characteristic of any substance that has activity in a biological system and/or organism. For instance, a substance that, when administered to an organism, has a biological effect on that organism, is considered to be biologically active. In particular embodiments, where a protein or polypeptide is biologically active, a portion of that protein or polypeptide that shares at least one biological activity of the protein or polypeptide is typically referred to as a “biologically active” portion.

Characteristic portion: As used herein, the term a “characteristic portion” of a substance, in the broadest sense, is one that shares some degree of sequence and/or structural identity and/or at least one functional characteristic with the relevant intact substance. For example, a “characteristic portion” of a protein or polypeptide is one that contains a continuous stretch of amino acids, or a collection of continuous stretches of amino acids, that together are characteristic of a protein or polypeptide. In some embodiments, each such continuous stretch generally will contain at least 2, 5, 10, 15, 20, 50, or more amino acids. A “characteristic portion” of a nucleic acid is one that contains a continuous stretch of nucleotides, or a collection of continuous stretches of nucleotides, that together are characteristic of a nucleic acid. In some embodiments, each such continuous stretch generally will contain at least 2, 5, 10, 15, 20, 50, or more nucleotides. In general, a characteristic portion of a substance (e.g. of a protein, nucleic acid, small molecule, etc.) is one that, in addition to the sequence and/or structural identity specified above, shares at least one functional characteristic with the relevant intact substance. In some embodiments, a characteristic portion may be biologically active.

Conjugated: As used herein, the terms “conjugated,” “linked,” and “attached,” when used with respect to two or more moieties, means that the moieties are physically associated or connected with one another, either directly or via one or more additional moieties that serves as a linking agent, to form a structure that is sufficiently stable so that the moieties remain physically associated under the conditions in which structure is used, e.g., physiological conditions. Typically the moieties are attached either by one or more covalent bonds or by a mechanism that involves specific binding. Alternately, a sufficient number of weaker interactions can provide sufficient stability for moieties to remain physically associated.

Functional: As used herein, a “functional” biological molecule is a biological molecule in a form in which it exhibits a property and/or activity by which it is characterized.

Homolog: As used herein, the term “homology” refers to the overall relatedness between polymeric molecules, e.g. between nucleic acid molecules (e.g. DNA molecules and/or RNA molecules) and/or between polypeptide molecules. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% similar.

Identity: As used herein, the term “identity” refers to the overall relatedness between polymeric molecules, e.g. between nucleic acid molecules (e.g. DNA molecules and/or RNA molecules) and/or between polypeptide molecules. Calculation of the percent identity of two nucleic acid sequences, for example, can be performed by aligning the two sequences for optimal comparison purposes (e.g. gaps can be introduced in one or both of a first and a second nucleic acid sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or substantially 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using the algorithm of Meyers and Miller (CABIOS, 1989, 4:11-17; incorporated herein by reference), which has been incorporated into the ALIGN program (version 2.0) using a PAM120 weight residue table, a gap length penalty of 12 and a gap penalty of 4. The percent identity between two nucleotide sequences can, alternatively, be determined using the GAP program in the GCG software package using an NWSgapdna.CMP matrix.

Inhibit expression of a gene: As used herein, the phrase “inhibit expression of a gene” means to cause a reduction in the amount of an expression product of the gene. The expression product can be an RNA transcribed from the gene (e.g. an mRNA) or a polypeptide translated from an mRNA transcribed from the gene. Typically a reduction in the level of an mRNA results in a reduction in the level of a polypeptide translated therefrom. The level of expression may be determined using standard techniques for measuring mRNA or protein.

In vitro: As used herein, the term “in vitro” refers to events that occur in an artificial environment, e.g., in a test tube or reaction vessel, in cell culture, etc., rather than within a multi-cellular organism.

In vivo: As used herein, the term “in vivo” refers to events that occur within a multi-cellular organism such as a non-human animal.

Isolated: As used herein, the term “isolated” refers to a substance and/or entity that has been (1) separated from at least some of the components with which it was associated when initially produced (whether in nature and/or in an experimental setting), and/or (2) produced, prepared, and/or manufactured by the hand of man. Isolated substances and/or entities may be separated from at least about 10%, about 20%, about 30%, about 40%, about 50%, about 60%, about 70%, about 80%, about 90%, or more of the other components with which they were initially associated. In some embodiments, isolated agents are more than about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components. As used herein, the term “isolated cell” refers to a cell not contained in a multi-cellular organism. In some embodiments, the term “isolated composition” refers to a composition present outside of a cell.

Liposomes: As used herein, the term “liposomes” refers to artificial microscopic spherical particles formed by a lipid-containing bilayer (or multilayers) enclosing an aqueous compartment.

microRNA (miRNA): As used herein, the term “microRNA” or “miRNA” refers to an RNAi agent that is approximately 21 nucleotides (nt)−23 nt in length. miRNAs can range between 18 nt-26 nt in length. Typically, miRNAs are single-stranded. However, in some embodiments, miRNAs may be at least partially double-stranded. In certain embodiments, miRNAs may comprise an RNA duplex (referred to herein as a “duplex region”) and may optionally further comprises one or two single-stranded overhangs. In some embodiments, an RNAi agent comprises a duplex region ranging from 15 bp to 29 bp in length and optionally further comprising one or two single-stranded overhangs. An miRNA may be formed from two RNA molecules that hybridize together, or may alternatively be generated from a single RNA molecule that includes a self-hybridizing portion. In general, free 5′ ends of miRNA molecules have phosphate groups, and free 3′ ends have hydroxyl groups. The duplex portion of an miRNA usually, but does not necessarily, comprise one or more bulges consisting of one or more unpaired nucleotides. One strand of an miRNA includes a portion that hybridizes with a target RNA. In certain embodiments, one strand of the miRNA is not precisely complementary with a region of the target RNA, meaning that the miRNA hybridizes to the target RNA with one or more mismatches. In some embodiments, one strand of the miRNA is precisely complementary with a region of the target RNA, meaning that the miRNA hybridizes to the target RNA with no mismatches. Typically, miRNAs are thought to mediate inhibition of gene expression by inhibiting translation of target transcripts. However, in some embodiments, miRNAs may mediate inhibition of gene expression by causing degradation of target transcripts.

Modulating Entity: As used herein, the term “modulating entity” refers to any entity that can be used to alter or affect delivery and/or efficacy of nanoparticles, protect nanoparticles while in transit, and/or control the delivery and/or efficacy of nanoparticles. In some embodiments, modulating entities can be used to alter or affect delivery and/or efficacy of agents; protect agents while in transit; and/or control the delivery and/or efficacy of agents. In some embodiments, modulating entities are any entities that alter or affect nanoparticle fate. For example, modulating entities may alter or affect the final tissue, cellular, or subcellular distribution of nanoparticles and/or agents. Alternatively or additionally, modulating entities may direct nanoparticles and/or agents to certain organs and/or tissues for excretion and/or breakdown. In some embodiments, modulating entities can protect nanoparticles, increase nanoparticle stability, increase nanoparticle half-life, increase nanoparticle circulation times, and/or combinations thereof. In certain embodiments, a modulating entity is polyethylene glycol. In certain embodiments, a modulating entity is a targeting moiety. In some embodiments, a modulating entity is a transfection reagent (e.g. dendrimer). In some embodiments, a modulating entity is a translocation entity. In some embodiments, a modulating entity is an entity that alters activity of an agent to be delivered. In some embodiments, a modulating entity is an entity that mediates controlled release of an agent. In certain embodiments, a modulating entity is an endosomal escape agent. In some embodiments, modulating entities are associated with nanoparticles. In some embodiments, modulating entities are associated with agents to be delivered. A modulating entity may be physically associated with the nanoparticle and/or agent to be delivered. In some embodiments, a modulating entity, agent, and/or nanoparticle are covalently or non-covalently conjugated to one another.

Nanoparticle: As used herein, the term “nanoparticle” refers to any particle having a diameter of less than 1000 nanometers (nm). In some embodiments, nanoparticles can be optically or magnetically detectable. In some embodiments, intrinsically fluorescent or luminescent nanoparticles, nanoparticles that comprise fluorescent or luminescent moieties, plasmon resonant nanoparticles, and magnetic nanoparticles are among the detectable nanoparticles that are used in various embodiments. In general, the nanoparticles should have dimensions small enough to allow their uptake by eukaryotic cells. Typically the nanoparticles have a longest straight dimension (e.g., diameter) of 200 nm or less. In some embodiments, the nanoparticles have a diameter of 100 nm or less. Smaller nanoparticles, e.g. having diameters of 50 nm or less, e.g., 5 nm-30 nm, are used in some embodiments. In certain embodiments, nanoparticles are quantum dots, i.e., bright, fluorescent nanocrystals with physical dimensions small enough such that the effect of quantum confinement gives rise to unique optical and electronic properties. In certain embodiments, optically detectable nanoparticles are metal nanoparticles. Metals of use in the nanoparticles include, but are not limited to, gold, silver, iron, cobalt, zinc, cadmium, nickel, gadolinium, chromium, copper, manganese, palladium, tin, and alloys and/or oxides thereof. In some embodiments, magnetic nanoparticles are of use in accordance with the invention. “Magnetic particles” refers to magnetically responsive particles that contain one or more metals or oxides or hydroxides thereof.

Nucleic acid: As used herein, the term “nucleic acid,” in its broadest sense, refers to any compound and/or substance that is or can be incorporated into an oligonucleotide chain. In some embodiments, a nucleic acid is a compound and/or substance that is or can be incorporated into an oligonucleotide chain via a phosphodiester linkage. In some embodiments, “nucleic acid” refers to individual nucleic acid residues (e.g. nucleotides and/or nucleosides). In some embodiments, “nucleic acid” refers to an oligonucleotide chain comprising individual nucleic acid residues. As used herein, the terms “oligonucleotide” and “polynucleotide” can be used interchangeably. In some embodiments, “nucleic acid” encompasses RNA as well as single and/or double-stranded DNA and/or cDNA. Furthermore, the terms “nucleic acid,” “DNA,” “RNA,” and/or similar terms include nucleic acid analogs, i.e. analogs having other than a phosphodiester backbone. For example, the so-called “peptide nucleic acids,” which are known in the art and have peptide bonds instead of phosphodiester bonds in the backbone, are considered within the scope of the present invention. The term “nucleotide sequence encoding an amino acid sequence” includes all nucleotide sequences that are degenerate versions of each other and/or encode the same amino acid sequence. Nucleotide sequences that encode proteins and/or RNA may include introns. Nucleic acids can be purified from natural sources, produced using recombinant expression systems and optionally purified, chemically synthesized, etc. Where appropriate, e.g. in the case of chemically synthesized molecules, nucleic acids can comprise nucleoside analogs such as analogs having chemically modified bases or sugars, backbone modifications, etc. A nucleic acid sequence is presented in the 5′ to 3′ direction unless otherwise indicated. The term “nucleic acid segment” is used herein to refer to a nucleic acid sequence that is a portion of a longer nucleic acid sequence. In many embodiments, a nucleic acid segment comprises at least 3, 4, 5, 6, 7, 8, 9, 10, or more residues. In some embodiments, a nucleic acid is or comprises natural nucleosides (e.g. adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages). In some embodiments, the present invention may be specifically directed to “unmodified nucleic acids,” meaning nucleic acids (e.g. polynucleotides and residues, including nucleotides and/or nucleosides) that have not been chemically modified in order to facilitate or achieve delivery.

Protein: As used herein, the term “protein” refers to a polypeptide (i.e., a string of at least two amino acids linked to one another by peptide bonds). Proteins may include moieties other than amino acids (e.g., may be glycoproteins, proteoglycans, etc.) and/or may be otherwise processed or modified. Those of ordinary skill in the art will appreciate that a “protein” can be a complete polypeptide chain as produced by a cell (with or without a signal sequence), or can be a characteristic portion thereof. Those of ordinary skill will appreciate that a protein can sometimes include more than one polypeptide chain, for example linked by one or more disulfide bonds or associated by other means. Polypeptides may contain L-amino acids, D-amino acids, or both and may contain any of a variety of amino acid modifications or analogs known in the art. Useful modifications include, e.g., terminal acetylation, amidation, etc. In some embodiments, proteins may comprise natural amino acids, non-natural amino acids, synthetic amino acids, and combinations thereof. The term “peptide” is generally used to refer to a polypeptide having a length of less than about 100 amino acids.

RNA interference (RNAi): As used herein, the term “RNA interference” or “RNAi” refers to sequence-specific inhibition of gene expression and/or reduction in target RNA levels mediated by an at least partly double-stranded RNA, which RNA comprises a portion that is substantially complementary to a target RNA. Typically, at least part of the substantially complementary portion is within the double stranded region of the RNA. In some embodiments, RNAi can occur via selective intracellular degradation of RNA. In some embodiments, RNAi can occur by translational repression.

RNAi agent: As used herein, the term “RNAi agent” refers to an RNA, optionally including one or more nucleotide analogs or modifications, having a structure characteristic of molecules that can mediate inhibition of gene expression through an RNAi mechanism. In some embodiments, RNAi agents mediate inhibition of gene expression by causing degradation of target transcripts. In some embodiments, RNAi agents mediate inhibition of gene expression by inhibiting translation of target transcripts. Generally, an RNAi agent includes a portion that is substantially complementary to a target RNA. In some embodiments, RNAi agents are at least partly double-stranded. In some embodiments, RNAi agents are single-stranded. In some embodiments, exemplary RNAi agents can include siRNA, shRNA, and/or miRNA. In some embodiments, RNAi agents may be composed entirely of natural RNA nucleotides (i.e., adenine, guanine, cytosine, and uracil). In some embodiments, RNAi agents may include one or more non-natural RNA nucleotides (e.g. nucleotide analogs, DNA nucleotides, etc.). Inclusion of non-natural RNA nucleic acid residues may be used to make the RNAi agent more resistant to cellular degradation than RNA. In some embodiments, the term “RNAi agent” may refer to any RNA, RNA derivative, and/or nucleic acid encoding an RNA that induces an RNAi effect (e.g. degradation of target RNA and/or inhibition of translation). In some embodiments, an RNAi agent may comprise a blunt-ended (i.e., without overhangs) dsRNA that can act as a Dicer substrate. For example, such an RNAi agent may comprise a blunt-ended dsRNA which is >25 base pairs length, which may optionally be chemically modified to abrogate an immune response.

RNAi-inducing entity: As used herein, the term “RNAi-inducing entity” encompasses any entity that delivers, regulates, and/or modifies the activity of an RNAi agent. In some embodiments, RNAi-inducing entities may include vectors (other than naturally occurring molecules not modified by the hand of man) whose presence within a cell results in RNAi and leads to reduced expression of a transcript to which the RNAi-inducing entity is targeted. In some embodiments, RNAi-inducing entities are RNAi-inducing vectors. In some embodiments, RNAi-inducing entities are compositions comprising RNAi agents and one or more pharmaceutically acceptable excipients and/or carriers.

RNAi-inducing vector: As used herein, the term “RNAi-inducing vector” refers to a vector whose presence within a cell results in production of one or more RNAs that self-hybridize or hybridize to each other to form an RNAi agent (e.g. siRNA, shRNA, and/or miRNA). In various embodiments, this term encompasses plasmids, e.g., DNA vectors (whose sequence may comprise sequence elements derived from a virus), or viruses (other than naturally occurring viruses or plasmids that have not been modified by the hand of man), whose presence within a cell results in production of one or more RNAs that self-hybridize or hybridize to each other to form an RNAi agent. In general, the vector comprises a nucleic acid operably linked to expression signal(s) so that one or more RNAs that hybridize or self-hybridize to form an RNAi agent are transcribed when the vector is present within a cell. Thus the vector provides a template for intracellular synthesis of the RNA or RNAs or precursors thereof. For purposes of inducing RNAi, presence of a viral genome in a cell (e.g., following fusion of the viral envelope with the cell membrane) is considered sufficient to constitute presence of the virus within the cell. In addition, for purposes of inducing RNAi, a vector is considered to be present within a cell if it is introduced into the cell, enters the cell, or is inherited from a parental cell, regardless of whether it is subsequently modified or processed within the cell. An RNAi-inducing vector is considered to be targeted to a transcript if presence of the vector within a cell results in production of one or more RNAs that hybridize to each other or self-hybridize to form an RNAi agent that is targeted to the transcript, i.e., if presence of the vector within a cell results in production of one or more RNAi agents targeted to the transcript.

Short RNAi agent: As used herein, the term “short RNAi agent” refers to an RNAi agent containing a dsRNA portion that is no greater than 50 base pairs in length, typically 30 base pairs or less in length, e.g., 17 base pairs-29 base pairs in length. The term “short RNAi agent” includes siRNA and shRNA.

Short, interfering RNA (siRNA): As used herein, the term “short, interfering RNA” or “siRNA” refers to an RNAi agent comprising an RNA duplex (referred to herein as a “duplex region”) that is approximately 19 basepairs (bp) in length and optionally further comprises one or two single-stranded overhangs. In some embodiments, an RNAi agent comprises a duplex region ranging from 15 bp to 29 bp in length and optionally further comprising one or two single-stranded overhangs. An siRNA may be formed from two RNA molecules that hybridize together, or may alternatively be generated from a single RNA molecule that includes a self-hybridizing portion. In general, free 5′ ends of siRNA molecules have phosphate groups, and free 3′ ends have hydroxyl groups. The duplex portion of an siRNA may, but typically does not, comprise one or more bulges consisting of one or more unpaired nucleotides. One strand of an siRNA includes a portion that hybridizes with a target RNA. In certain embodiments, one strand of the siRNA is precisely complementary with a region of the target RNA, meaning that the siRNA hybridizes to the target RNA without a single mismatch. In some embodiments, one or more mismatches between the siRNA and the targeted portion of the target RNA may exist. In some embodiments in which perfect complementarity is not achieved, any mismatches are generally located at or near the siRNA termini. In some embodiments, siRNAs mediate inhibition of gene expression by causing degradation of target transcripts.

Short hairpin RNA (shRNA): As used herein, the term “short hairpin RNA” or “shRNA” refers to an RNAi agent comprising an RNA having at least two complementary portions hybridized or capable of hybridizing to form a double-stranded (duplex) structure sufficiently long to mediate RNAi (typically at least approximately 19 bp in length), and at least one single-stranded portion, typically ranging between approximately 1 nucleotide (nt) and approximately 10 nt in length that forms a loop. In some embodiments, an shRNA comprises a duplex portion ranging from 15 bp to 29 bp in length and at least one single-stranded portion, typically ranging between approximately 1 nt and approximately 10 nt in length that forms a loop. The duplex portion may, but typically does not, comprise one or more bulges consisting of one or more unpaired nucleotides. In some embodiments, siRNAs mediate inhibition of gene expression by causing degradation of target transcripts. shRNAs are thought to be processed into siRNAs by the conserved cellular RNAi machinery. Thus shRNAs may be precursors of siRNAs. Regardless, siRNAs in general are capable of inhibiting expression of a target RNA, similar to siRNAs.

Small Molecule: In general, a “small molecule” is understood in the art to be an organic molecule that is less than about 5 kilodaltons (Kd) in size. In some embodiments, the small molecule is less than about 4 Kd, about 3 Kd, about 2 Kd, or about 1 Kd. In some embodiments, the small molecule is less than about 800 daltons (D), about 600 D, about 500 D, about 400 D, about 300 D, about 200 D, or about 100 D. In some embodiments, a small molecule is less than about 2000 g/mol, less than about 1500 g/mol, less than about 1000 g/mol, less than about 800 g/mol, or less than about 500 g/mol. In some embodiments, small molecules are non-polymeric. In some embodiments, small molecules are not proteins, peptides, or amino acids. In some embodiments, small molecules are not nucleic acids or nucleotides. In some embodiments, small molecules are not saccharides or polysaccharides.

Specific binding: As used herein, the term “specific binding” refers to non-covalent physical association of a first and a second moiety wherein the association between the first and second moieties is at least 100 times as strong as the association of either moiety with most or all other moieties present in the environment in which binding occurs. Binding of two or more entities may be considered specific if the equilibrium dissociation constant, K_{d}, is 10⁻⁶ M or less, 10⁻⁷ M or less, 10⁻³ M or less, or 10⁻⁹ M or less under the conditions employed, e.g. under physiological conditions such as those inside a cell or consistent with cell survival. Examples of specific binding interactions include antibody-antigen interactions, avidin-biotin interactions, hybridization between complementary nucleic acids, etc.

Subject: As used herein, the term “subject” or “patient” refers to any organism to which compositions in accordance with the invention may be administered, e.g., for experimental, diagnostic, prophylactic, and/or therapeutic purposes. Typical subjects include animals (e.g., mammals such as mice, rats, rabbits, non-human primates, and humans; insects; worms; etc.).

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Suffering from: An individual who is “suffering from” a disease, disorder, and/or condition has been diagnosed with or displays one or more symptoms of the disease, disorder, and/or condition.

Susceptible to: An individual who is “susceptible to” a disease, disorder, and/or condition has not been diagnosed with the disease, disorder, and/or condition. In some embodiments, an individual who is susceptible to a disease, disorder, and/or condition may not exhibit symptoms of the disease, disorder, and/or condition. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

Target gene: As used herein, the term “target gene” refers to any gene whose expression is inhibited by an RNAi agent.

Target transcript: As used herein, the term “target transcript” refers to any mRNA transcribed from a target gene.

Transfection reagent: As used herein, the term “transfection reagent” refers to any substance that enhances the transfer or uptake of an exogenous nucleic acid into a cell when the cell is contacted with the nucleic acid in the presence of the transfection reagent. In some embodiments, transfection reagents enhance the transfer of an exogenous nucleic acid, e.g. RNA, into mammalian cells.

Therapeutically effective amount: As used herein, the term “therapeutically effective amount” of a therapeutic agent means an amount that is sufficient, when administered to a subject suffering from or susceptible to a disease, disorder, and/or condition, to treat, diagnose, prevent, and/or delay the onset of the symptom(s) of the disease, disorder, and/or condition.

Therapeutic agent: As used herein, the phrase “therapeutic agent” refers to any agent that, when administered to a subject, has a therapeutic effect and/or elicits a desired biological and/or pharmacological effect.

Treating: As used herein, the term “treat,” “treatment,” or “treating” refers to any method used to partially or completely alleviate, ameliorate, relieve, inhibit, prevent, delay onset of, reduce severity of and/or reduce incidence of one or more symptoms or features of a particular disease, disorder, and/or condition. Treatment may be administered to a subject who does not exhibit signs of a disease and/or exhibits only early signs of the disease for the purpose of decreasing the risk of developing pathology associated with the disease.

Unnatural amino acid: As used herein, the term “unnatural amino acid” refers to any amino acid other than the 20 naturally-occurring amino acids found in naturally occurring proteins, and includes amino acid analogues. In general, any compound that can be incorporated into a polypeptide chain can be an unnatural amino acid. In some embodiments, such compounds have the chemical structure H₂N—CHR—CO₂H. The alpha-carbon may be in the L-configuration, as in naturally occurring amino acids, or may be in the D-configuration.

### DETAILED DESCRIPTION OF CERTAIN EMBODIMENTS OF THE INVENTION

The present invention encompasses the recognition that modulating entities can be used to alter delivery and/or activity of nanoparticles, protect nanoparticles while in transit, and/or control the delivery and/or activity of nanoparticles. In some embodiments, such nanoparticles are used for the delivery of agents to tissues, cells, and/or subcellular locales. Thus, the present invention encompasses the recognition that modulating entities can be used to alter delivery, activity, and/or release of agents; protect agents while in transit; and/or control the delivery, activity, and/or release of agents. In some embodiments, modulating entities are any entities that alter or affect nanoparticle fate. For example, modulating entities may alter or affect the final tissue, cellular, or subcellular distribution of nanoparticles and/or agents. Alternatively or additionally, modulating entities may direct nanoparticles and/or agents to certain organs and/or tissues for excretion and/or breakdown.

In some embodiments, the present invention provides for uptake of RNA by particular eukaryotic tissues, cells, and/or subcellular locales. A variety of different classes of RNA molecules can be delivered. For example, the RNA may be a short RNAi agent such as an siRNA that inhibits gene expression or may be a transfer RNA (tRNA) that functions in protein synthesis. In certain embodiments, the amount of RNA delivered to the interior of a cell serves as an indicator of the activity of the RNA in the cell. For example, in certain embodiments, RNA uptake correlates with the activity of the RNA in the cell.

In some embodiments, methods in accordance with the present invention involve contacting a cell or, more typically, a plurality of cells, with a nanoparticle, e.g., an optically or magnetically detectable nanoparticle associated with a modulating entity. The nanoparticle may be further associated with one or more agents to be delivered. In some embodiments, the nanoparticle has dimensions small enough to allow it to enter the cell; in some embodiments, the nanoparticle is delivered to the interior of the cell. Delivery of an agent can be achieved in any of a number of ways as discussed further below.

In certain embodiments, a cell or plurality of cells is contacted with a plurality of nanoparticles comprising or consisting of nanoparticles that have one or more optical and/or magnetic properties. In some embodiments, a population of nanoparticles has substantially uniform optical and/or magnetic properties so that, for example, the population can be distinguished from a different population of nanoparticles and/or from other entities. Typically, individual particles of a population having substantially uniform optical or magnetic properties will be substantially similar in size, shape, and/or composition. When cells are contacted with a population of nanoparticles, the magnitude of the signal acquired from a particular cell is, on the average, indicative of the number of nanoparticles taken up by the cell. Suitable nanoparticles include, e.g. quantum dots (QDs), fluorescent or luminescent nanoparticles, and magnetic nanoparticles.

In certain embodiments, nanoparticles are associated with one or more agents to be delivered to the tissue, cell, and/or subcellular location. The number of nanoparticles taken up by the cell is positively correlated with the amount of agent taken up by the cell. In other words, if the number of nanoparticles present in two cells is compared, the cell that contains a larger number of nanoparticles typically contains a larger amount of agent. The correlation between nanoparticle and agent uptake can be linear or non-linear and can exist over all or part of a range of nanoparticle and/or agent concentrations to which a cell is exposed. In certain embodiments, the nanoparticle and the agent are physically associated, so that they are taken up together. For example, the nanoparticle and the agent may be associated in a complex with a transfection reagent. In certain embodiments, the transfection reagent both enhances uptake of the nanoparticle and the agent by the cell and serves to physically associate the nanoparticle and the agent with one another. In some embodiments, the nanoparticle and agent to be delivered do not remain associated throughout delivery. In some embodiments, the nanoparticle and agent are delivered together; in some embodiments, the nanoparticle and agent are not delivered together.

As described in Examples 1 and 2, using a QD/agent co-delivery technique in accordance with the invention, cellular fluorescence was shown to correlate with level of activity of the agent, allowing collection of a uniformly silenced cell population by fluorescence-activated cell sorting (FACS). The present invention demonstrates that the presence of optically detectable nanoparticles such as QDs within mammalian cells does not interfere with the activity of an agent even when the particles are present in large numbers. The superior brightness and photostability of QD probes in cells sustained not only FACS, but also live imaging, and immunostaining procedures. As described in Example 3, with the use of two QD colors and two siRNAs, the method was used to generate cell populations with multiplexed levels of knockdown. Example 4 shows that a homogenously silenced cell population generated using this method is essential to observing the phenotypic effects of decreased T-cadherin protein expression on cell-cell communication between hepatocytes and non-parenchymal cells, thus providing a sample of the wide range of biologically relevant discoveries that are made possible by the methods in accordance with the invention.

As described in Example 5, QDs demonstrate superior photostability and brightness relative to fluorescent dyes for siRNA tracking. Uptake and silencing activity of quantum dot/agent complexes is demonstrated in Example 6, and targeted delivery of QDs to cells is shown in Example 7.

As described in Example 9, photosensitizers can effectively induce endosomal escape when combined with targeting peptide. A targeting peptide was conjugated to fluorescein (i.e., a photosensitizer) and incubated with glioblastoma cells. After light irradiation for three minutes, fluorescence of the peptide was more evenly distributed inside cells, indicating endosomal escape of the targeting peptide.

As described in Example 10, an agent and targeting peptide are conjugated to nanoparticles via protease-cleavable peptides. Proteases such as matrix metalloproteases (MMPs) are upregulated in many types of tumors. Therefore, agents to be delivered that are conjugated to nanoparticle entities via protease-cleavable bonds are released from nanoparticles when nanoparticles reach tumor sites in vivo.

As described in Example 11, multifunctional nanoparticles are multivalent, can be remotely actuated, and imaged noninvasively in vivo. Superparamagnetic nanoparticles embedded in tissue transduce external electromagnetic energy to heat, thereby melting oligonucleotide duplexes that act as heat-labile tethers to model drugs. In vitro, nanoparticles hybridized to fluorescein-conjugated 18mer were embedded in hydrogel plugs. In vivo, application of EMF to implanted phantoms with 18mer tethers resulted in release of model drugs and penetration into surrounding tissue. Nanoparticle conjugates comprising heat-labile tethers (i.e. “thermally-responsive linkers”) are described in further detail in co-pending U.S. patent application entitled “REMOTELY TRIGGERED RELEASE FROM HEATABLE SURFACES,” filed Dec. 6, 2007 (the entire contents of which are incorporated herein by reference and are attached hereto as Appendix A).

As described in Example 12, when siRNA is associated with nanoparticles and polyethylene glycol (PEG), siRNA degradation can be reduced. PEG can be utilized to protect siRNA from serum nucleases by providing steric hindrance to prevent nuclease binding to siRNA.

As described in Example 13, the present inventors recognize that the ability to reveal bioactive domains on the surface of nanoparticles in response to microenvironmental cues in tumors could provide a powerful means for targeting their activity. Example 13 demonstrates the feasibility of such a design by veiling nanoparticles with protease-removable polymer coatings. Multimodal visualization and quantification of this model system establishes the utility of these coatings to improve nanoparticle delivery and direct the unveiling of bioactive surface groups in the tumor.

### Nanoparticles

In some embodiments, nanoparticles useful in accordance with the present invention are biodegradable and/or biocompatible. In general, a biocompatible substance is not toxic to cells. In some embodiments, a substance is considered to be biocompatible if its addition to cells results in less than a certain threshhold of cell death (e.g. about 50%, about 45%, about 40%, about 35%, about 30%, about 25%, about 20%, about 15%, about 10%, about 5%, or less than about 5% cell death). In some embodiments, a substance is considered to be biocompatible if its addition to cells does not induce adverse effects. In general, a biodegradable substance is one that undergoes breakdown under physiological conditions over the course of a therapeutically relevant time period (e.g., weeks, months, or years). In some embodiments, a biodegradable substance is a substance that can be broken down by cellular machinery. In some embodiments, a biodegradable substance is a substance that can be broken down by chemical processes.

In some embodiments, a particle which is biocompatible and/or biodegradable may be associated with a modulating entity and/or an agent to be delivered that is not biocompatible, is not biodegradable, or is neither biocompatible nor biodegradable. In some embodiments, a particle which is biocompatible and/or biodegradable may be associated with a modulating entity and/or an agent to be delivered is also biocompatible and/or biodegradable.

In general, a particle in accordance with the present invention is any entity having a greatest dimension (e.g. diameter) of less than 100 microns (μm). In some embodiments, particles have a greatest dimension of less than 10 μm. In some embodiments, particles have a greatest dimension of less than 1000 nanometers (nm). In some embodiments, particles have a greatest dimension of less than 900 nm, 800 nm, 700 nm, 600 nm, 500 nm, 400 nm, 300 nm, 200 nm, or 100 nm. Typically, particles have a greatest dimension (e.g. diameter) of 300 nm or less. In some embodiments, particles have a greatest dimension (e.g., diameter) of 250 nm or less. In some embodiments, particles have a greatest dimension (e.g., diameter) of 200 nm or less. In some embodiments, particles have a greatest dimension (e.g., diameter) of 150 nm or less. In some embodiments, particles have a greatest dimension (e.g., diameter) of 100 nm or less. Smaller particles, e.g., having a greatest dimension of 50 nm or less are used in some embodiments of the invention. In some embodiments, particles have a greatest dimension ranging between 5 nm and 1 μm. In some embodiments, particles have a greatest dimension ranging between 25 nm and 200 nm.

In some embodiments, particles have a diameter of approximately 1000 nm. In some embodiments, particles have a diameter of approximately 750 nm. In some embodiments, particles have a diameter of approximately 500 nm. In some embodiments, particles have a diameter of approximately 450 nm. In some embodiments, particles have a diameter of approximately 400 nm. In some embodiments, particles have a diameter of approximately 350 nm. In some embodiments, particles have a diameter of approximately 300 nm. In some embodiments, particles have a diameter of approximately 275 nm. In some embodiments, particles have a diameter of approximately 250 nm. In some embodiments, particles have a diameter of approximately 225 nm. In some embodiments, particles have a diameter of approximately 200 nm. In some embodiments, particles have a diameter of approximately 175 nm. In some embodiments, particles have a diameter of approximately 150 nm. In some embodiments, particles have a diameter of approximately 125 nm. In some embodiments, particles have a diameter of approximately 100 nm. In some embodiments, particles have a diameter of approximately 75 nm. In some embodiments, particles have a diameter of approximately 50 nm. In some embodiments, particles have a diameter of approximately 25 nm.

In certain embodiments, particles are greater in size than the renal excretion limit (e.g. particles having diameters of greater than 6 nm). In specific embodiments, particles have diameters greater than 5 nm, greater than 10 nm, greater than 15 nm, greater than 20 nm, greater than 50 nm, greater than 100 nm, greater than 250 nm, greater than 500 nm, greater than 1000 nm, or larger. In certain embodiments, particles are small enough to avoid clearance of particles from the bloodstream by the liver (e.g. particles having diameters of less than 1000 nm). In specific embodiments, particles have diameters less than 1500 nm, less than 1000 nm, less than 750 nm, less than 500 nm, less than 250 nm, less than 100 nm, or smaller. In general, physiochemical features of particles, including particle size, can be selected to allow a particle to circulate longer in plasma by decreasing renal excretion and/or liver clearance. In some embodiments, particles have diameters ranging from 5 nm to 1500 nm, from 5 nm to 1000 nm, from 5 nm to 750 nm, from 5 nm to 500 nm, from 5 nm to 250 nm, or from 5 nm to 100 nm. In some embodiments, particles have diameters ranging from 10 nm to 1500 nm, from 15 nm to 1500 nm, from 20 nm to 1500 nm, from 50 nm to 1500 nm, from 100 nm to 1500 nm, from 250 nm to 1500 nm, from 500 nm to 1500 nm, or from 1000 nm to 1500 nm. In some embodiments, particles under 100 nm may be easily endocytosed in the reticuloendothelial system (RES). In some embodiments, particles under 400 nm may be characterized by enhanced accumulation in tumors. While not wishing to be bound by any theory, enhanced accumulation in tumors may be caused by the increased permeability of angiogenic tumor vasculature relative to normal vasculature. Particles can diffuse through such “leaky” vasculature, resulting in accumulation of particles in tumors.

It is often desirable to use a population of particles that is relatively uniform in terms of size, shape, and/or composition so that each particle has similar properties. For example, at least 80%, at least 90%, or at least 95% of the particles may have a diameter or greatest dimension that falls within 5%, 10%, or 20% of the average diameter or greatest dimension. In some embodiments, a population of particles may be heterogeneous with respect to size, shape, and/or composition.

Zeta potential is a measurement of surface potential of a particle. In some embodiments, particles have a zeta potential ranging between −50 mV and +50 mV. In some embodiments, particles have a zeta potential ranging between −25 mV and +25 mV. In some embodiments, particles have a zeta potential ranging between −10 mV and +10 mV. In some embodiments, particles have a zeta potential ranging between −5 mV and +5 mV. In some embodiments, particles have a zeta potential ranging between 0 mV and +50 mV. In some embodiments, particles have a zeta potential ranging between 0 mV and +25 mV. In some embodiments, particles have a zeta potential ranging between 0 mV and +10 mV. In some embodiments, particles have a zeta potential ranging between 0 mV and +5 mV. In some embodiments, particles have a zeta potential ranging between −50 mV and 0 mV. In some embodiments, particles have a zeta potential ranging between −25 mV and 0 mV. In some embodiments, particles have a zeta potential ranging between −10 mV and 0 mV. In some embodiments, particles have a zeta potential ranging between −5 mV and 0 mV. In some embodiments, particles have a substantially neutral zeta potential (i.e. approximately 0 mV).

Particles can have a variety of different shapes including spheres, oblate spheroids, cylinders, ovals, ellipses, shells, cubes, cuboids, cones, pyramids, rods (e.g., cylinders or elongated structures having a square or rectangular cross-section), tetrapods (particles having four leg-like appendages), triangles, prisms, etc.

In some embodiments, particles are microparticles (e.g. microspheres). In general, a “microparticle” refers to any particle having a diameter of less than 1000 μm. In some embodiments, particles are nanoparticles (e.g. nanospheres). In general, a “nanoparticle” refers to any particle having a diameter of less than 1000 nm. In some embodiments, particles are picoparticles (e.g. picospheres). In general, a “picoparticle” refers to any particle having a diameter of less than 1 nm. In some embodiments, particles are liposomes. In some embodiments, particles are micelles.

Particles can be solid or hollow and can comprise one or more layers (e.g., nanoshells, nanorings, etc.). Particles may have a core/shell structure, wherein the core(s) and shell(s) can be made of different materials. Particles may comprise gradient or homogeneous alloys. Particles may be composite particles made of two or more materials, of which one, more than one, or all of the materials possesses magnetic properties, electrically detectable properties, and/or optically detectable properties.

In certain embodiments of the invention, a particle is porous, by which is meant that the particle contains holes or channels, which are typically small compared with the size of a particle. For example a particle may be a porous silica particle, e.g. a mesoporous silica nanoparticle or may have a coating of mesoporous silica (Lin et al., 2005, J. Am. Chem. Soc., 17:4570). Particles may have pores ranging from about 1 nm to about 50 nm in diameter, e.g. between about 1 nm and 20 nm in diameter. Between about 10% and 95% of the volume of a particle may consist of voids within the pores or channels.

Particles may have a coating layer. Use of a biocompatible coating layer can be advantageous, e.g., if the particles contain materials that are toxic to cells. Suitable coating materials include, but are not limited to, natural proteins such as bovine serum albumin (BSA), biocompatible hydrophilic polymers such as polyethylene glycol (PEG) or a PEG derivative, phospholipid-(PEG), silica, lipids, polymers, carbohydrates such as dextran, other nanoparticles that can be associated with inventive nanoparticles etc. Coatings may be applied or assembled in a variety of ways such as by dipping, using a layer-by-layer technique, by self-assembly, conjugation, etc. Self-assembly refers to a process of spontaneous assembly of a higher order structure that relies on the natural attraction of the components of the higher order structure (e.g., molecules) for each other. It typically occurs through random movements of the molecules and formation of bonds based on size, shape, composition, or chemical properties.

In some embodiments, particles may optionally comprise one or more dispersion media, surfactants, release-retarding ingredients, or other pharmaceutically acceptable excipient. In some embodiments, particles may optionally comprise one or more plasticizers or additives.

A variety of different nanoparticles are of use in accordance with the invention. In some embodiments, polymeric particles may be used in accordance with the present invention. For example, C32 is a polymer that may be used in accordance with the present invention. Alternatively or additionally, Duncan (2003, Nat. Rev. Drug Discov., 2:347; incorporated herein by reference) and Moghimi et al., (2001, Pharmacol. Rev., 53:283; incorporated herein by reference) describe polymers that can be of use in accordance with the present invention.

Non-Polymeric Particles

In some embodiments, particles may be intrinsically magnetic particles. In some embodiments, fluorescent or luminescent nanoparticles, particles that comprise fluorescent or luminescent moieties, and plasmon resonant particles are among the particles that are used in various embodiments of the invention. In some embodiments, the nanoparticles have detectable optical and/or magnetic properties. An optically detectable nanoparticle is one that can be detected within a living cell using optical means compatible with cell viability. Optical detection is accomplished by detecting the scattering, emission, and/or absorption of light that falls within the optical region of the spectrum, i.e., that portion of the spectrum extending from approximately 180 nm to several microns. Optionally a sample containing cells is exposed to a source of electromagnetic energy. In some embodiments, absorption of electromagnetic energy (e.g. light of a given wavelength) by the nanoparticle or a component thereof is followed by the emission of light at longer wavelengths, and the emitted light is detected. In some embodiments, scattering of light by the nanoparticles is detected. In certain embodiments, light falling within the visible portion of the electromagnetic spectrum, i.e., the portion of the spectrum that is detectable by the human eye (approximately 400 nm to approximately 700 nm) is detected. In some embodiments, light that falls within the infrared or ultraviolet region of the spectrum is detected.

The optical property can be a feature of an absorption, emission, or scattering spectrum or a change in a feature of an absorption, emission, or scattering spectrum. The optical property can be a visually detectable feature such as, for example, color, apparent size, or visibility (i.e. simply whether or not the particle is visible under particular conditions). Features of a spectrum include, for example, peak wavelength or frequency (wavelength or frequency at which maximum emission, scattering intensity, extinction, absorption, etc. occurs), peak magnitude (e.g., peak emission value, peak scattering intensity, peak absorbance value, etc.), peak width at half height, or metrics derived from any of the foregoing such as ratio of peak magnitude to peak width. Certain spectra may contain multiple peaks, of which one is typically the major peak and has significantly greater intensity than the others. Each spectral peak has associated features. Typically, for any particular spectrum, spectral features such as peak wavelength or frequency, peak magnitude, peak width at half height, etc., are determined with reference to the major peak. The features of each peak, number of peaks, separation between peaks, etc., can be considered to be features of the spectrum as a whole. The foregoing features can be measured as a function of the direction of polarization of light illuminating the particles; thus polarization dependence can be measured. Features associated with hyper-Rayleigh scattering can be measured. Fluorescence detection can include detection of fluorescence modes.

Intrinsically fluorescent or luminescent nanoparticles, nanoparticles that comprise fluorescent or luminescent moieties, plasmon resonant nanoparticles, and magnetic nanoparticles are among the detectable nanoparticles that are used in various embodiments in accordance with the invention. Such particles can have a variety of different shapes including spheres, oblate spheroids, cylinders, shells, cubes, pyramids, rods (e.g., cylinders or elongated structures having a square or rectangular cross-section), tetrapods (particles having four leg-like appendages), triangles, prisms, etc.

In general, the nanoparticles should have dimensions small enough to allow their uptake by eukaryotic cells. Typically the nanoparticles have a longest straight dimension (e.g., diameter) of 200 nm or less. In some embodiments, the nanoparticles have a diameter of 100 nm or less. Smaller nanoparticles, e.g. having diameters of 50 nm or less, e.g., 5 nm-30 nm, are used in some embodiments in accordance with the invention. In some embodiments, the term “nanoparticle” encompasses atomic clusters, which have a typical diameter of 1 nm or less and generally contain from several (e.g., 3-4) up to several hundred atoms.

In some embodiments, nanoparticles larger than 5 nm may reduce clearance by the kidney. In some embodiments, nanoparticles under 100 nm may be easily endocytosed in the reticuloendothelial system (RES). In some embodiments, nanoparticles under 400 nm may be characterized by enhanced accumulation in tumors. While not wishing to be bound by any theory, enhanced accumulation in tumors may be caused by the increased permeability of angiogenic tumor vasculature relative to normal vasculature. Nanoparticles can diffuse through such “leaky” vasculature, resulting in accumulation of nanoparticles in tumors.

The nanoparticles can be solid or hollow and can comprise one or more layers (e.g., nanoshells, nanorings). They may have a core/shell structure, wherein the core(s) and shell(s) can be made of different materials. In certain embodiments, they are composed of either gradient or homogeneous alloys. In certain embodiments, nanoparticles are composite particles made of two or more materials, of which one, more than one, or all of the materials possesses an optically or magnetically detectable property.

It is often desirable to use a population of nanoparticles that is relatively uniform in terms of size, shape, and/or composition so that each particle has similar properties, e.g. similar optical or magnetic properties. For example, at least 80%, at least 90%, or at least 95% of the particles may have a diameter or longest straight line dimension that falls within 5%, 10%, or 20% of the average diameter or longest straight line dimension.

In certain embodiments, one or more substantially uniform populations of particles is used, e.g., 2, 3, 4, 5, or more substantially uniform populations having distinguishable optical and/or magnetic properties. Each population of particles is associated with an agent. Use of multiple distinguishable particle populations allows tracking of multiple different agents concurrently. It will be appreciated that a combination of two or more populations having distinguishable properties can be considered to be a single population. It will further be appreciated that combining two or more populations of particles in different ratios can expand the range of coding possibilities (see, e.g. Mattheakis et al., 2004, Anal. Biochem., 327:200; incorporated herein by reference). In some embodiments, the present invention encompasses any suitable means of relating the identity of an agent to a population of nanoparticles such that detecting the nanoparticles in a cell is indicative of the presence of the agent in a cell.

Nanoparticles comprising one or more optically or magnetically detectable materials may have a coating layer. Use of a biocompatible coating layer can be advantageous, e.g., if the particles contain materials that are toxic to cells. In some embodiments, coatings may be useful for protecting the agent to be delivered (e.g. to protect an RNAi entity to be delivered from serum nucleases). Suitable coating materials include, but are not limited to, proteins such as bovine serum albumin (BSA), polyethylene glycol (PEG) or a PEG derivative, phospholipid-(PEG), silica, lipids, carbohydrates such as dextran, etc. Coatings may be applied or assembled in a variety of ways such as by dipping, using a layer-by-layer technique, by self-assembly, etc. Self-assembly refers to a process of spontaneous assembly of a higher order structure that relies on the natural attraction of the components of the higher order structure (e.g., molecules) for each other. It typically occurs through random movements of the molecules and formation of bonds based on size, shape, composition or chemical properties.

In certain embodiments, nanoparticles are quantum dots (QDs). QDs are bright, fluorescent nanocrystals with physical dimensions small enough such that the effect of quantum confinement gives rise to unique optical and electronic properties. Semiconductor QDs are often composed of atoms from groups II-VI or III-V in the periodic table, but other compositions are possible (see, e.g. Zheng et al., 2004, Phys. Rev. Lett., 93(7); incorporated herein by reference; describing gold QDs). By varying their size and composition, the emission wavelength can be tuned (i.e., adjusted in a predictable and controllable manner) from the blue to the near infrared. QDs generally have a broad absorption spectrum and a narrow emission spectrum. Thus different QDs having distinguishable optical properties (e.g., peak emission wavelength) can be excited using a single source. QDs are brighter than most conventional fluorescent dyes by approximately 10-fold (Wu et al., 2003, Nat. Biotechnol., 21:41; and Gao et al., 2004, Nat. Biotechnol., 22:969; both of which are incorporated herein by reference) and have been significantly easier to detect than GFP among background autofluorescence in vivo (Gao et al., 2004, Nat. Biotechnol., 22:969; incorporated herein by reference). Furthermore, QDs are far less susceptible to photobleaching, fluorescing more than 20 times longer than conventional fluorescent dyes under continuous mercury lamp exposure (Derfus et al., 2004, Adv. Mat., 16:961; incorporated herein by reference).

QDs and methods for their synthesis are well known in the art (see, e.g. U.S. Pat. Nos. 6,322,901; 6,576,291; and 6,815,064; all of which are incorporated herein by reference). QDs can be rendered water soluble by applying coating layers comprising a variety of different materials (see, e.g. U.S. Pat. Nos. 6,423,551; 6,251,303; 6,319,426; 6,426,513; 6,444,143; and 6,649,138; all of which are incorporated herein by reference). For example, QDs can be solubilized using amphiphilic polymers. Exemplary polymers that have been employed include octylamine-modified low molecular weight polyacrylic acid, polyethylene-glycol (PEG)-derivatized phospholipids, polyanhydrides, block copolymers, etc. (Gao, 2004, Nat. Biotechnol., 22:969; incorporated herein by reference). QDs can be conjugated with a variety of different biomolecules such as nucleic acids, polypeptides, antibodies, streptavidin, lectins, and polysaccharides, e.g. via any of a number of different functional groups or linking agents that can be directly or indirectly linked to a coating layer (see, e.g. U.S. Pat. Nos. 5,990,479; 6,207,392; 6,251,303; 6,306,610; 6,325,144; and 6,423,551; all of which are incorporated herein by reference).

The inventors and others have shown that QDs can be rendered non-cytotoxic (Derfus et al., 2004, Nano Letters, 4:11; incorporated herein by reference) and innocuous to normal cell physiology and common cellular assays, such as immunostaining and reporter gene expression (Mattheakis et al., 2004, Anal. Biochem., 327:200; incorporated herein by reference). For example, QDs can be coated with PEG as described in Example 1 (e.g., Derfus et al., 2004, Adv. Mat., 16:961; incorporated herein by reference). In some embodiments, QDs are encapsulated with a high molecular weight ABC triblock copolymer (Gao, 2004, Nat. Biotechnol., 22:969; incorporated herein by reference). Features and uses of QDs, optionally modified with affinity agents such as antibodies, have been reviewed (see, e.g. Alivisatos et al., 2005, Ann. Rev. Biomed. Eng., 7:55; and Hotz, 2005, Methods Mol. Biol., 303: 1; both of which are incorporated herein by reference). QDs with a wide variety of absorption and emission spectra are commercially available, e.g., from Quantum Dot Corp. (Hayward Calif.; now owned by Invitrogen) or from Evident Technologies (Troy, N.Y.). For example, QDs having peak emission wavelengths of approximately 525 nm, approximately 535 nm, approximately 545 nm, approximately 565 nm, approximately 585 nm, approximately 605 nm, approximately 655 nm, approximately 705 nm, and approximately 800 nm are available. Thus QDs can have a range of different colors across the visible portion of the spectrum and in some cases even beyond.

Fluorescence or luminescence can be detected using any approach known in the art including, but not limited to, spectrometry, fluorescence microscopy, flow cytometry, etc. Spectrofluorometers and microplate readers are typically used to measure average properties of a sample while fluorescence microscopes resolve fluorescence as a function of spatial coordinates in two or three dimensions for microscopic objects (e.g., less than approximately 0.1 mm diameter). Microscope-based systems are thus suitable for detecting and optionally quantitating nanoparticles inside individual cells.

Flow cytometry measures properties such as light scattering and/or fluorescence on individual cells in a flowing stream, allowing subpopulations within a sample to be identified, analyzed, and optionally quantitated (see, e.g., Mattheakis et al., 2004, Analytical Biochemistry, 327:200; Chattopadhyay et al., 2006, Nat. Med., 12:972; incorporated herein by reference). Multiparameter flow cytometers are available. In certain embodiments, laser scanning cytometery is used (Kamentsky, 2001, Methods Cell Biol., 63:51; incorporated herein by reference). Laser scanning cytometry can provide equivalent data to a flow cytometer but is typically applied to cells on a solid support such as a slide. It allows light scatter and fluorescence measurements and records the position of each measurement. Cells of interest may be re-located, visualized, stained, analyzed, and/or photographed. Laser scanning cytometers are available, e.g., from CompuCyte (Cambridge, Mass.).

In certain embodiments, imaging systems comprising an epifluorescence microscope equipped with a laser (e.g., a 488 nm argon laser) for excitation and appropriate emission filter(s) are used. The filters should allow discrimination between different populations of nanoparticles used in the particular assay. For example, in some embodiments, the microscope is equipped with fifteen 10 nm bandpass filters spaced to cover portion of the spectrum between 520 nm and 660 nm, which would allow the detection of a wide variety of different fluorescent particles. Fluorescence spectra can be obtained from populations of nanoparticles using a standard UV/visible spectrometer.

In certain embodiments, optically detectable nanoparticles are metal nanoparticles. Metals of use in the nanoparticles include, but are not limited to, gold, silver, iron, cobalt, zinc, cadmium, nickel, gadolinium, chromium, copper, manganese, palladium, tin, and alloys thereof. Oxides of any of these metals can be used.

Noble metals (e.g., gold, silver, copper, platinum, palladium) are often used for plasmon resonant particles, which are discussed in further detail below. For example, gold, silver, or an alloy comprising gold, silver, and optionally one or more other metals can be used. Core/shell particles (e.g., having a silver core with an outer shell of gold, or vice versa) can be used. Particles containing a metal core and a nonmetallic inorganic or organic outer shell, or vice versa, can be used. In certain embodiments, the nonmetallic core or shell comprises or consists of a dielectric material such as silica. Composite particles in which a plurality of metal particles are embedded or trapped in a nonmetal (e.g., a polymer or a silica shell) may be used. Hollow metal particles (e.g., hollow nanoshells) having an interior space or cavity are used in some embodiments. In some embodiments, a nanoshell comprising two or more concentric hollow spheres is used. Such a nanoparticle optionally comprises a core, e.g. made of a dielectric material.

In certain embodiments, at least 1%, or typically at least 5%, of the mass or volume of the particle or number of atoms in the particle is contributed by metal atoms. In certain embodiments, the amount of metal in the particle, or in a core or coating layer comprising a metal, ranges from approximately 5% to 100% by mass, volume, or number of atoms, or can assume any value or range between 5% and 100%.

Certain metal nanoparticles, referred to as plasmon resonant particles, exhibit the well known phenomenon of plasmon resonance. When a metal nanoparticle (usually made of a noble metal such as gold, silver, copper, platinum, etc.) is subjected to an external electric field, its conduction electrons are displaced from their equilibrium positions with respect to the nuclei, which in turn exert an attractive, restoring force. If the electric field is oscillating (as in the case of electromagnetic radiation such as light), the result is a collective oscillation of the conduction electrons in the nanoparticle, known as plasmon resonance (Kelly et al., 2003, J. Phys. Chem. B., 107:668; Schultz et al., 2000, Proc. Natl. Acad. Sci., USA, 97:996; and Schultz, 2003, Curr. Op. Biotechnol., 14:13; all of which are incorporated herein by reference). The plasmon resonance phenomenon results in extremely efficient wavelength-dependent scattering and absorption of light by the particles over particular bands of frequencies, often in the visible range. Scattering and absorption give rise to a number of distinctive optical properties that can be detected using various approaches including visually (i.e., by the naked eye or using appropriate microscopic techniques) and/or by obtaining a spectrum, e.g. a scattering spectrum, extinction (scattering+absorption) spectrum, or absorption spectrum from the particle(s).

The features of the spectrum of a plasmon resonant particle (e.g., peak wavelength) depend on a number of factors, including the particle's material composition, the shape and size of the particle, the refractive index or dielectric properties of the surrounding medium, and the presence of other particles in the vicinity. Selection of particular particle shapes, sizes, and compositions makes it possible to produce particles with a wide range of distinguishable optically detectable properties thus allowing for concurrent detection of multiple RNAs by using particles with different properties such as peak scattering wavelenth.

Single plasmon resonant nanoparticles of sufficient size can be individually detected using a variety of approaches. For example, particles larger than about 30 nm in diameter are readily detectable under an optical microscope operating in dark-field. A spectrum from these particles can be obtained, e.g., using a CCD detector or other optical detection device. Despite their small dimensions relative to the wavelength of light, metal nanoparticles can be detected optically because they scatter light very efficiently at their plasmon resonance frequency. An 80 nm particle, for example, would be millions of times brighter than a fluorescein molecule under the same illumination conditions (Schultz et al., 2000, Proc. Natl. Acad. Sci., USA, 97:996; incorporated herein by reference). Individual plasmon resonant particles can be optically detected using a variety of approaches including near-field scanning optical microscopy, differential interference microscopy with video enhancement, total internal reflection microscopy, photo-thermal interference contrast, etc. For measurements on a population of cells, a standard spectrometer, e.g. equipped for detection of UV, visible, and/or infrared light, can be used. In certain embodiments, nanoparticles are optically detected with the use of surface-enhanced Raman scattering (SERS) (Jackson and Halas, 2004, Proc. Natl. Acad. Sci., USA, 101:17930; incorporated herein by reference). Optical properties of metal nanoparticles and methods for synthesis of metal nanoparticles have been reviewed (Link and El-Sayed, 2003, Ann. Rev. Phys. Chem., 54:331; and Masala and Seshadri, 2004, Ann. Rev. Mater. Res., 34:41; both of which are incorporated herein by reference).

Certain lanthanide ion-doped nanoparticles exhibit strong fluorescence and are of use in certain embodiments. A variety of different dopant molecules can be used. For example, fluorescent europium-doped yttrium vanadate (YVO₄) nanoparticles have been produced (Beaureparie et al., 2004, Nano Letters, 4:2079; incorporated herein by reference). These nanoparticles may be synthesized in water and are readily functionalized with biomolecules.

In some embodiments, magnetic nanoparticles are of use in accordance with the invention. “Magnetic particles” refers to magnetically responsive particles that contain one or more metals or oxides or hydroxides thereof. Such particles typically react to magnetic force resulting from a magnetic field. The field can attract or repel the particle towards or away from the source of the magnetic field, respectively, optionally causing acceleration or movement in a desired direction in space. A magnetically detectable nanoparticle is a magnetic particle that can be detected within a living cell as a consequence of its magnetic properties. Magnetic particles may comprise one or more ferrimagnetic, ferromagnetic, paramagnetic, and/or superparamagnetic materials. Useful particles may be made entirely or in part of one or more materials selected from the group consisting of: iron, cobalt, nickel, niobium, magnetic iron oxides, hydroxides such as maghemite (γ-Fe₂O₃), magnetite (Fe₃O₄), feroxyhyte (FeO(OH)), double oxides or hydroxides of two- or three-valent iron with two- or three-valent other metal ions such as those from the first row of transition metals such as Co(II), Mn(II), Cu(II), Ni(II), Cr(III), Gd(III), Dy(III), Sm(III), mixtures of the afore-mentioned oxides or hydroxides, and mixtures of any of the foregoing. See, e.g., U.S. Pat. No. 5,916,539 (incorporated herein by reference) for suitable synthesis methods for certain of these particles. Additional materials that may be used in magnetic particles include yttrium, europium, and vanadium.

A magnetic particle may contain a magnetic material and one or more nonmagnetic materials, which may be a metal or a nonmetal. In certain embodiments, the particle is a composite particle comprising an inner core or layer containing a first material and an outer layer or shell containing a second material, wherein at least one of the materials is magnetic. Optionally both of the materials are metals. In some embodiments, the nanoparticle is an iron oxide nanoparticle, e.g. the particle has a core of iron oxide. Optionally the iron oxide is monocrystalline. In some embodiment, the nanoparticle is a superparamagnetic iron oxide nanoparticle, e.g. the particle has a core of superparamagnetic iron oxide.

Detection of magnetic nanoparticles may be performed using any method known in the art. For example, a magnetometer or a detector based on the phenomenon of magnetic resonance (NMR) can be employed. Superconducting quantum interference devices (SQUID), which use the properties of electron-pair wave coherence and Josephson junctions to detect very small magnetic fields can be used. Magnetic force microscopy or handheld magnetic readers can be used. U.S Patent Publication 2003/009029 (incorporated herein by reference) describes various suitable methods. Magnetic resonance microscopy offers one approach (Wind et al., 2000, J. Magn. Reson., 147:371; incorporated herein by reference).

In certain embodiments, the nanoparticle comprises a bulk material that is not intrinsically fluorescent, luminescent, plasmon resonant, or magnetic. The nanoparticle comprises one or more fluorescent, luminescent, or magnetic moieties. For example, the nanoparticle may comprise QDs, fluorescent or luminescent organic molecules, or smaller particles of a magnetic material. In some embodiments, an optically detectable moiety such as a fluorescent or luminescent dye, etc., is entrapped, embedded, or encapsulated by a nanoparticle core and/or coating layer.

In certain embodiments, the nanoparticle comprises silica (SiO₂). For example, the nanoparticle may consist at least in part of silica, e.g. it may consist essentially of silica or may have an optional coating layer composed of a different material. In some embodiments, the particle has a silica core and an outside layer composed of one or more other materials. In some embodiments, the particle has an outer layer of silica and a core composed of one or more other materials. The amount of silica in the particle, or in a core or coating layer comprising silica, can range from approximately 5% to 100% by mass, volume, or number of atoms, or can assume any value or range between 5% and 100%.

Silica-containing nanoparticles may be made by a variety of methods. Certain of these methods utilize the Stöber synthesis which involves hydrolysis of tetraethoxyorthosilicate (TEOS) catalyzed by ammonia in water/ethanol mixtures, or variations thereof. Microemulsion procedures can be used. For example, a water-in-oil emulsion in which water droplets are dispersed as nanosized liquid entities in a continuous domain of oil and surfactants and serve as nanoreactors for nanoparticle synthesis offer a convenient approach. Silica nanoparticles can be functionalized with biomolecules such as polypeptides and/or “doped” or “loaded” with certain inorganic or organic fluorescent dyes (see, e.g. U.S. Patent Publication 2004/0067503; Bagwe et al., 2004, Langmuir, 20:8336; Van Blaaderen and Vrij, 1992, Langmuir, 8:2921; Lin et al., 2005, J. Am. Chem. Soc., 17:4570; Zhao et al., 2004, Adv. Mat., 16:173; and Wang et al., 2005, Nano Letters, 5:37; all of which are incorporated herein by reference).

In certain embodiments, the particle is made at least in part of a porous material, by which is meant that the material contains many holes or channels, which are typically small compared with the size of the particle. For example the particle may be a porous silica nanoparticle, e.g., a mesoporous silica nanoparticle or may have a coating of mesoporous silica (Lin et al., 2005, J. Am. Chem. Soc., 17:4570; incorporated herein by reference). The particles may have pores ranging in diameter from about 1 nm to about 50 nm in diameter, e.g. between about 1 nm and 20 nm in diameter. Between about 20% and 95% of the volume of the particle may consist of empty space within the pores or channels.

In some embodiments, a nanoparticle composed in part or essentially consisting of an organic polymer is used. A wide variety of organic polymers and methods for forming nanoparticles therefrom are known in the art. For example, particles composed at least in part of polymethylmethacrylate, polyacrylamide, poly(vinyl chloride), carboxylated poly(vinyl chloride), or poly(vinyl chloride-co-vinyl acetate-co-vinyl alcohol) may be used. Optionally the nanoparticle comprises one or more plasticizers or additives. Co-polymers, block co-polymers, and/or grafted co-polymers can be used.

Fluorescent and luminescent moieties include a variety of different organic or inorganic small molecules commonly referred to as “dyes,” “labels,” or “indicators.” Examples include fluorescein, rhodamine, acridine dyes, Alexa dyes, cyanine dyes, etc. Fluorescent and luminescent moieties may include a variety of naturally occurring proteins and derivatives thereof, e.g., genetically engineered variants. For example, fluorescent proteins include green fluorescent protein (GFP), enhanced GFP, red, blue, yellow, cyan, and sapphire fluorescent proteins, reef coral fluorescent protein, etc. Luminescent proteins include luciferase, aequorin and derivatives thereof. Numerous fluorescent and luminescent dyes and proteins are known in the art (see, e.g. Valeur, B., “Molecular Fluorescence: Principles and Applications,” John Wiley and Sons, 2002; Handbook of Fluorescent Probes and Research Products, Molecular Probes, 9^{th} edition, 2002; and The Handbook-A Guide to Fluorescent Probes and Labeling Technologies, Invitrogen, 10^{th} edition, available at the Invitrogen web site).

### Modulating Entities

The present invention provides nanoparticles to be delivered that are associated with one or more entities that modulate delivery and/or activity of nanoparticles, protect nanoparticles while in transit, and/or control the delivery and/or activity of nanoparticles. The present invention provides agents to be delivered that are associated with one or more entities that modulate delivery, activity, and/or release of agents, protect agents while in transit, and/or control the delivery, activity, and/or release of agents. The modulating entity may be physically associated with the nanoparticle and/or agent. In some embodiments, the modulating entity, nanoparticle and/or agent are either covalently or non-covalently conjugated to one another.

In accordance with the present invention, the modulating entity may be any entity that alters or affects the efficiency, specificity, and/or accuracy of delivery or activity of the nanoparticle. In some embodiments, the modulating entity alters delivery or activity of the nanoparticle, protects the nanoparticle while in transit, and/or controls the delivery or activity of the nanoparticle. Alternatively or additionally, in those embodiments in which the nanoparticle is also associated with one or more agents, the modulating entity may enhance delivery or activity of the agent, protect the agent and/or control the delivery or activity of the agent.

In certain embodiments, the modulating entity may be selected from the group consisting of targeting entities, transfection reagents, translocation entities, endosome escape entities, entities that alter activity of an agent, entities that mediate controlled release of an agent, etc.

Targeting Entities

In some embodiments, a modulating entity in accordance with the present invention is or comprises a targeting entity. In general, a targeting entity is any entity that binds to a component associated with an organ, tissue, cell, subcellular locale, and/or extracellular matrix component. In some embodiments, such a component is referred to as a “target” or a “marker,” and these are discussed in further detail below.

A targeting entity may be a nucleic acid, polypeptide, glycoprotein, carbohydrate, lipid, etc. For example, a targeting entity can be a nucleic acid targeting entity (e.g. an aptamer) that binds to a cell type specific marker. In general, an aptamer is an oligonucleotide (e.g., DNA, RNA, or an analog or derivative thereof) that binds to a particular target, such as a polypeptide. In some embodiments, a targeting entity may be a naturally occurring or synthetic ligand for a cell surface receptor, e.g., a growth factor, hormone, LDL, transferrin, etc. A targeting entity can be an antibody, which term is intended to include antibody fragments, characteristic portions of antibodies, single chain antibodies, etc. Synthetic binding proteins such as affibodies, etc., can be used. Peptide targeting entities can be identified, e.g., using procedures such as phage display. This widely used technique has been used to identify cell specific ligands for a variety of different cell types.

In some embodiments, targeting entities bind to an organ, tissue, cell, extracellular matrix component, and/or intracellular compartment that is associated with a specific developmental stage or a specific disease state (i.e. a “target” or “marker”). In some embodiments, a target is an antigen on the surface of a cell, such as a cell surface receptor, an integrin, a transmembrane protein, an ion channel, and/or a membrane transport protein. In some embodiments, a target is an intracellular protein. In some embodiments, a target is a soluble protein, such as immunoglobulin. In some embodiments, a target is more prevalent, accessible, and/or abundant in a diseased locale (e.g. organ, tissue, cell, subcellular locale, and/or extracellular matrix component) than in a healthy locale. To give but one example, in some embodiments, a target is preferentially expressed in tumor tissues versus normal tissues. In some embodiments, a target is more prevalent, accessible, and/or abundant in locales (e.g. organs, tissues, cells, subcellular locales, and/or extracellular matrix components) associated with a particular developmental state than in locales associated with a different developmental state. In some embodiments, targeting entities facilitate the passive entry into target sites by extending circulation time of conjugates, reducing non-specific clearance of conjugates, and/or geometrically enhancing the accumulation of conjugates in target sites.

In certain embodiments, the marker may be expressed in significant amounts mainly on one or a few cell types or in one or a few diseases. A cell type specific marker for a particular cell type is expressed at levels at least 3 fold greater in that cell type than in a reference population of cells which may consist, for example, of a mixture containing cells from a plurality (e.g., 5-10 or more) of different tissues or organs in approximately equal amounts. In some embodiments, the cell type specific marker is present at levels at least 4-fold, between 5-10 fold, or more than 10-fold greater than its average expression in a reference population. Detection or measurement of a cell type specific marker may make it possible to distinguish the cell type or types of interest from cells of many, most, or all other types.

In some embodiments, a targeting entity in accordance with the present invention may be a nucleic acid. As used herein, a “nucleic acid targeting entity” refers to a nucleic acid that binds selectively to a target. In some embodiments, a nucleic acid targeting entity is a nucleic acid aptamer. An aptamer is typically a polynucleotide that binds to a specific target structure that is associated with a particular organ, tissue, cell, subcellular locale, and/or extracellular matrix component. In general, the targeting function of the aptamer is based on the three-dimensional structure of the aptamer and/or target.

In some embodiments, a targeting entity in accordance with the present invention may be a small molecule. In certain embodiments, small molecules are less than about 2000 g/mol in size. In some embodiments, small molecules are less than about 1500 g/mol or less than about 1000 g/mol. In some embodiments, small molecules are less than about 800 g/mol or less than about 500 g/mol. One of ordinary skill in the art will appreciate that any small molecule that specifically binds to a desired target can be used in accordance with the present invention.

In some embodiments, a targeting entity in accordance with the present invention may be a protein or peptide. In certain embodiments, peptides range from about 5 to 100, 10 to 75, 15 to 50, or 20 to 25 amino acids in size. In some embodiments, a peptide sequence can be based on the sequence of a protein. In some embodiments, a peptide sequence can be a random arrangement of amino acids.

The terms “polypeptide” and “peptide” are used interchangeably herein, with “peptide” typically referring to a polypeptide having a length of less than about 100 amino acids. Polypeptides may contain L-amino acids, D-amino acids, or both and may contain any of a variety of amino acid modifications or analogs known in the art. Useful modifications include, e.g., terminal acetylation, amidation, lipidation, phosphorylation, glycosylation, acylation, farnesylation, sulfation, etc.

Exemplary proteins that may be used as targeting moieties in accordance with the present invention include, but are not limited to, antibodies, receptors, cytokines, peptide hormones, proteins derived from combinatorial libraries (e.g. avimers, affibodies, etc.), and characteristic portions thereof.

In some embodiments, a targeting entity may be an antibody and/or characteristic portion thereof. The term “antibody” refers to any immunoglobulin, whether natural or wholly or partially synthetically produced and to derivatives thereof and characteristic portions thereof. An antibody may be monoclonal or polyclonal. An antibody may be a member of any immunoglobulin class, including any of the human classes: IgG, IgM, IgA, IgD, and IgE.

As used herein, an antibody fragment (i.e. characteristic portion of an antibody) refers to any derivative of an antibody which is less than full-length. In general, an antibody fragment retains at least a significant portion of the full-length antibody's specific binding ability. Examples of antibody fragments include, but are not limited to, Fab, Fab′, F(ab′)2, scFv, Fv, dsFv diabody, and Fd fragments.

An antibody fragment may be produced by any means. For example, an antibody fragment may be enzymatically or chemically produced by fragmentation of an intact antibody and/or it may be recombinantly produced from a gene encoding the partial antibody sequence. Alternatively or additionally, an antibody fragment may be wholly or partially synthetically produced. An antibody fragment may optionally comprise a single chain antibody fragment. Alternatively or additionally, an antibody fragment may comprise multiple chains which are linked together, for example, by disulfide linkages. An antibody fragment may optionally comprise a multimolecular complex. A functional antibody fragment will typically comprise at least about 50 amino acids and more typically will comprise at least about 200 amino acids.

In some embodiments, antibodies may include chimeric (e.g. “humanized”) and single chain (recombinant) antibodies. In some embodiments, antibodies may have reduced effector functions and/or bispecific molecules. In some embodiments, antibodies may include fragments produced by a Fab expression library.

Single-chain Fvs (scFvs) are recombinant antibody fragments consisting of only the variable light chain (VL) and variable heavy chain (VH) covalently connected to one another by a polypeptide linker. Either VL or VH may comprise the NH2-terminal domain. The polypeptide linker may be of variable length and composition so long as the two variable domains are bridged without significant steric interference. Typically, linkers primarily comprise stretches of glycine and serine residues with some glutamic acid or lysine residues interspersed for solubility.

Diabodies are dimeric scFvs. Diabodies typically have shorter peptide linkers than most scFvs, and they often show a preference for associating as dimers.

An Fv fragment is an antibody fragment which consists of one VH and one VL domain held together by noncovalent interactions. The term “dsFv” as used herein refers to an Fv with an engineered intermolecular disulfide bond to stabilize the VH-VL pair.

A F(ab′)2 fragment is an antibody fragment essentially equivalent to that obtained from immunoglobulins by digestion with an enzyme pepsin at pH 4.0-4.5. The fragment may be recombinantly produced.

A Fab′ fragment is an antibody fragment essentially equivalent to that obtained by reduction of the disulfide bridge or bridges joining the two heavy chain pieces in the F(ab′)2 fragment. The Fab′ fragment may be recombinantly produced.

A Fab fragment is an antibody fragment essentially equivalent to that obtained by digestion of immunoglobulins with an enzyme (e.g. papain). The Fab fragment may be recombinantly produced. The heavy chain segment of the Fab fragment is the Fd piece.

In some embodiments, a targeting entity in accordance with the present invention may comprise a carbohydrate (e.g. glycoproteins, proteoglycans, etc.). In some embodiments, a carbohydrate may be a polysaccharide comprising simple sugars (or their derivatives) connected by glycosidic bonds, as known in the art. Such sugars may include, but are not limited to, glucose, fructose, galactose, ribose, lactose, sucrose, maltose, trehalose, cellobiose, mannose, xylose, arabinose, glucoronic acid, galactoronic acid, mannuronic acid, glucosamine, galatosamine, and neuramic acid. In some embodiments, a carbohydrate may be one or more of pullulan, cellulose, microcrystalline cellulose, hydroxypropyl methylcellulose, hydroxycellulose, methylcellulose, dextran, cyclodextran, glycogen, starch, hydroxyethylstarch, carageenan, glycon, amylose, chitosan, N,O-carboxylmethylchitosan, algin and alginic acid, starch, chitin, heparin, konjac, glucommannan, pustulan, heparin, hyaluronic acid, curdlan, and xanthan. In some embodiments, the carbohydrate may be aminated, carboxylated, acetylated and/or sulfated. In some embodiments, hydrophilic polysaccharides can be modified to become hydrophobic by introducing a large number of side-chain hydrophobic groups.

In some embodiments, a targeting entity in accordance with the present invention may comprise one or more fatty acid groups or salts thereof (e.g. lipoproteins). In some embodiments, a fatty acid group may comprise digestible, long chain (e.g. C₈-C₅₀), substituted or unsubstituted hydrocarbons. In some embodiments, a fatty acid group may be a C₁₀-C₂₀ fatty acid or salt thereof. In some embodiments, a fatty acid group may be a C₁₅-C₂₀ fatty acid or salt thereof. In some embodiments, a fatty acid group may be a C₁₅-C₂₅ fatty acid or salt thereof. In some embodiments, a fatty acid group may be unsaturated. In some embodiments, a fatty acid group may be monounsaturated. In some embodiments, a fatty acid group may be polyunsaturated. In some embodiments, a double bond of an unsaturated fatty acid group may be in the cis conformation. In some embodiments, a double bond of an unsaturated fatty acid may be in the trans conformation. In some embodiments, a fatty acid group may be one or more of butyric, caproic, caprylic, capric, lauric, myristic, palmitic, stearic, arachidic, behenic, or lignoceric acid. In some embodiments, a fatty acid group may be one or more of palmitoleic, oleic, vaccenic, linoleic, alpha-linoleic, gamma-linoleic, arachidonic, gadoleic, arachidonic, eicosapentaenoic, docosahexaenoic, or erucic acid.

In some embodiments, nanoparticle entities are not targeted to particular locales (e.g. organs, tissues, cells, subcellular locales, and/or extracellular matrix components) by any of the targeting entities described above. In some embodiments, targeting may instead be facilitated by a property intrinsic to a nanoparticle entity (e.g. geometry of the nanoparticle entity and/or assembly of multiple nanoparticle entities).

In some embodiments, an agent to be delivered may function as a targeting entity as described herein. To give but one example, an antibody that is useful for targeting inventive conjugates to specific tissues may also serve as a therapeutic agent. In some embodiments, the agent to be delivered may be distinct from a targeting entity.

Numerous markers are known in the art. Typical markers include cell surface proteins, e.g. receptors. Exemplary receptors include, but are not limited to, the transferrin receptor; LDL receptor; growth factor receptors such as epidermal growth factor receptor family members (e.g., EGFR, HER-2, HER-3, HER-4, HER-2/neu) or vascular endothelial growth factor receptors; cytokine receptors; cell adhesion molecules; integrins; selectins; CD molecules; etc. The marker can be a molecule that is present exclusively or in higher amounts on a malignant cell, e.g. a tumor antigen. For example, prostate-specific membrane antigen (PSMA) is expressed at the surface of prostate cancer cells. In certain embodiments, the marker is an endothelial cell marker.

In certain embodiments, the marker is a tumor marker. The marker may be a polypeptide that is expressed at higher levels on dividing than on non-dividing cells. Nucleolin is an example. The peptide known as F3 is a suitable targeting agent for directing a nanoparticle to nucleolin (Porkka et al., 2002, Proc. Natl. Acad. Sci., USA, 99:444; Christian et al. 2003, J. Cell Biol., 163:871; both of which are incorporated herein by reference). As described in Example 6, conjugating nanoparticles (QDs) with peptide F3 was shown to improve nanoparticle uptake by tumor cells.

It will be appreciated that various changes in the amino acid sequence of a peptide, such as an endosome disrupting peptide, translocation peptide, cell targeting peptide, etc., can be made without substantially affecting the function of the peptide. For example, 1, 2, 3, or more such changes such as deletions, insertions, substitutions, etc. may be made. Typically the resulting peptide will have at least 80% sequence identity, e.g., 90% sequence identity, with the original peptide. Such variations are within the scope of the invention.

FIG. 10 presents a schematic diagram illustrating multifunctional nanoparticles for siRNA delivery in some embodiments. The particles, which are optionally optically or magnetically detectable, contain a core and a coating layer. The surface of the particles is functionalized with a targeting peptide, an endosomal escape peptide, and an agent to be delivered. The targeting entity binds to a cell surface marker that is selectively present on malignant cells. The particle is internalized and enters the endosome. The agent is released from the particle, optionally as a result of cleavage of a labile bond such as a disulfide, and the agent is released from the endosome into the cytoplasm, where it functions in a therapeutically useful manner. The optically or magnetically detectable nanoparticle can be detected to provide an indication of cellular uptake of the agent and/or its activity. The method thus facilitates evaluating the efficacy of different agents, different delivery vehicles, etc. The method is of use to guide dosing for therapy of a disease that is treated by the agent.

Transfection Reagents

In certain embodiments, one or more transfection reagents are employed to alter intracellular delivery of a nanoparticle and/or agent to be delivered. The present invention demonstrates the formation of complexes comprising a transfection reagent, a nanoparticle, and an agent to be delivered. In certain embodiments, the agent is a functional RNA, such as an siRNA. Notably, the invention further demonstrates that such complexes can be efficiently delivered to the interior of mammalian cells and that the siRNA can effectively mediate gene silencing following internalization.

A variety of different transfection reagents are of use in accordance with the invention. A number of transfection reagents have been developed to alter delivery of large DNA molecules (typically several hundred to thousands of base pairs in length), which differ significantly in terms of structure from small RNA species such as short RNAi agents and tRNAs. Nevertheless, certain of these transfection reagents mediate intracellular delivery of short RNAi agents and/or tRNAs.

A transfection reagent of use in accordance with the present invention may contain one or more naturally occurring, synthetic, and/or derivatized lipids. Cationic and/or neutral lipids or mixtures thereof may be used. Many cationic lipids are amphiphilic molecules containing a positively charged polar headgroup linked (e.g. via an anchor) to a hydrophobic domain often comprising two alkyl chains. Structural variations include the length and degree of unsaturation of the alkyl chains (Elouhabi and Ruysschaert, 2005, Mol. Ther., 11:336; and Heyes et al., 2005, J. Cont. Rel., 107:276; both of which are incorporated herein by reference). Cationic lipids include, for example, dimyristyl oxypropyl-3-dimethylhydroxy ethylammonium bromide (DMRIE), dilauryl oxypropyl-3-dimethylhydroxy ethylammonium bromide (DLRIE), N-[1-(2,3-dioleoyloxyl) propal]-n,n,n-trimethylammonium sulfate (DOTAP), dioleoylphosphatidylethanolamine (DOPE), dipalmitoylethylphosphatidylcholine (DPEPC), dioleoylphosphatidylcholine (DOPC), lipopolylysine, didoceyl methylammonium bromide (DDAB), 2,3-dioleoyloxy-N-[2-(sperminecarboxamidoet-hyl]-N,N-di-methyl-1-propanaminium trifluoroacetate (DOSPA), cetyltrimethylammonium bromide (CTAB), beta.-[N,(N′,N′-dimethylaminoethane)-carbamoyl] cholesterol (DC-Cholesterol, also known as DC-Chol), (-alanyl cholesterol, N-[1-(2,3-dioleoyloxy)propyl]-N,N,N-trimethylammonium chloride (DOTMA), N¹-cholesteryloxycarbonyl-3,7-diazanonane-1,9-diamine (CDAN), dipalmitoylphosphatidylethanolamine—5-carboxyspermylamide (DPPES), dicaproylphosphatidylethanolamine (DCPE), 4-dimethylaminopyridine (DMAP), dimyristoylphosphatidylethanolamine (DMPE), dioleoylethylphosphocholine (DOEPC), dioctadecylamidoglycyl spermidine (DOGS), and N-[1-(2,3-dioleoyloxy)propyl]-N-[1-(2-hydroxyethyl)]-N,N-dimethylammonium iodide (DOHME). Some representative cationic lipids include, but are not limited to, phosphatidylethanolamine, phospatidylcholine, glycero-3-ethylphosphatidyl-choline and fatty acyl esters thereof, di- and trimethyl ammonium propane, di- and tri-ethylammonium propane and fatty acyl esters thereof, e.g., N-[1-(2,3-dioleoyloxy)propyl]-N,N-,N-trimethylammonium chloride (DOTMA).

A variety of proprietary transfection reagents, most of which comprise one or more lipids, available commercially from suppliers such as Invitrogen (Carlsbad, Calif.), Quiagen (Valencia, Calif.), Promega (Madison, Wis.), etc., may be used. Examples include Lipofectin®, Lipofectamine®, Lipofectamine 2000®, Optifect®, Cytofectin®, Transfectace®, Transfectam®, Cytofectin®, Oligofectamine®, Effectene®, etc. A variety of transfection reagents have been developed or optimized for delivery of siRNA to mammalian cells. Examples include X-tremeGENE siRNA Transfection Reagent (Roche Applied Science), silMPORTER™ siRNA Transfection Reagent (Upstate), BLOCK-iT™ Technology (Invitrogen), RNAiFect Reagent (QIAGEN), GeneEraser™ siRNA Transfection Reagent (Stratagene), RiboJuice™ siRNA Transfection Reagent (Novagen), EXPRESS-si Delivery Kit (Genospectra, Inc.), HiPerFect Transfection Reagent (QIAGEN), siPORT™, siPORT™ lipid, siPORT™ amine (all from Ambion), DharmaFECT™ (Dharmacon), etc.

Cationic polymers may be used as transfection reagents in accordance with the present invention. Exemplary cationic polymers include polyethylenimine (PEI), polylysine (PLL), polyarginine (PLA), polyvinylpyrrolidone (PVP), chitosan, protamine, polyphosphates, polyphosphoesters (see U.S. Pat. No. 6,852,709; incorporated herein by reference), poly(N-isopropylacrylamide), etc. Certain of these polymers comprise primary amine groups, imine groups, guanidine groups, and/or imidazole groups. Some examples include poly(β-amino ester) (PAE) polymers (such as those described in U.S. Pat. No. 6,998,115 and U.S. Patent Publication 2004/0071654; both of which are incorporated herein by reference). The cationic polymer may be linear or branched. Blends, copolymers, and modified cationic polymers can be used. In certain embodiments, a cationic polymer having a molecular weight of at least about 25 kD is used. In some embodiments, deacylated PEI is used. For example, residual N-acyl moieties can be removed from commercially available PEI, or PEI can be synthesized, e.g., by acid-catalyzed hydrolysis of poly(2-ethyl-2-oxazoline), to yield the pure polycations (88).

Dendrimers are of use as transfection reagents in accordance with the present invention. Dendrimers are polymers that are synthesized as approximately spherical structures typically ranging from 1 nm to about 20 nm in diameter having a center from which chains extend in a tree-like, branching morphology. Molecular weight and the number of terminal groups increase exponentially as a function of generation (the number of layers) of the polymer. Different types of dendrimers can be synthesized based on different core structures. Dendrimers suitable for use in accordance with the present invention include, but are not limited to, polyamidoamine (PAMAM), polypropylamine (POPAM), polyethylenimine, iptycene, aliphatic poly(ether), and/or aromatic polyether dendrimers (see, e.g. U.S. Pat. No. 6,471,968; Derfus et al., 2004, Adv. Mat., 16:961; and Boas and Heegaard, 2004, Chem. Soc. Rev., 33:43; all of which are incorporated herein by reference).

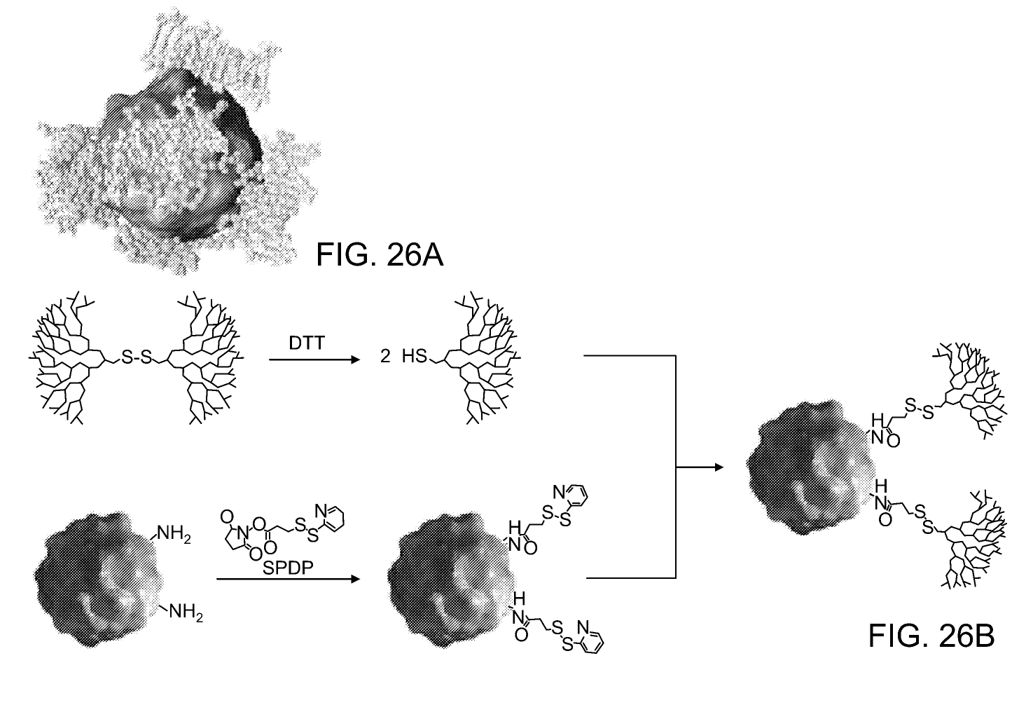

In some embodiments, dendrimers may be associated with nanoparticles comprising a magnetic core (see, e.g. FIG. 26). In some embodiments, such association may be non-covalent (e.g. affinity interactions, metal coordination, physical adsorption, host-guest interactions, hydrophobic interactions, pi stacking interactions, hydrogen bonding interactions, van der Waals interactions, magnetic interactions, electrostatic interactions, dipole-dipole interactions, etc.). In some embodiments, such association may be covalent. In some embodiments, covalent association is mediated by a linker, as described herein. In some embodiments, covalent association is mediated by a cleavable linker, as described herein.

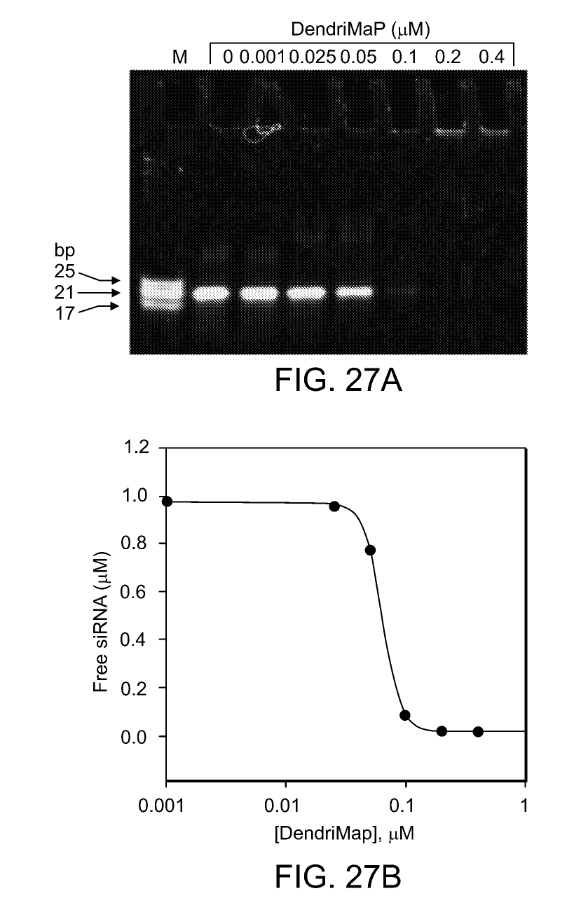

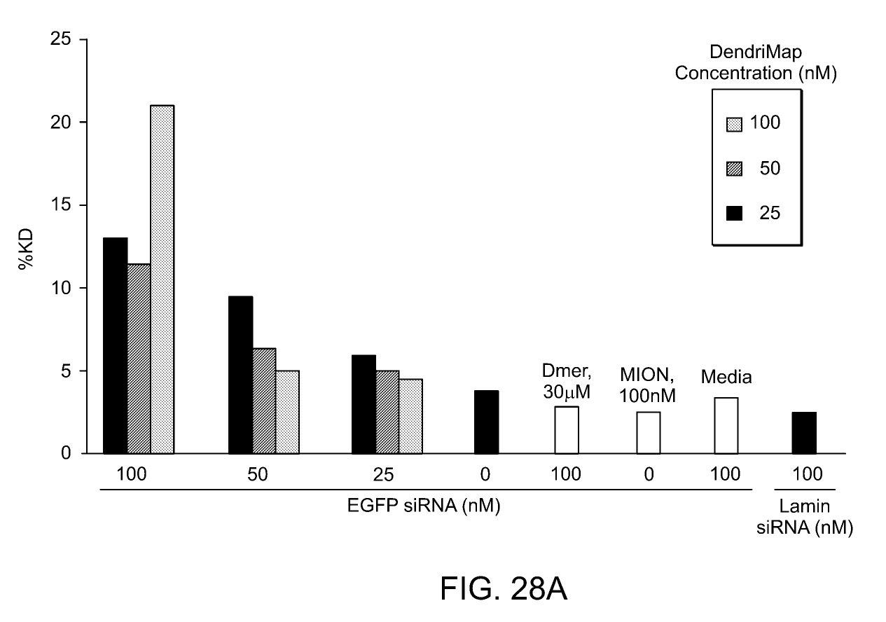

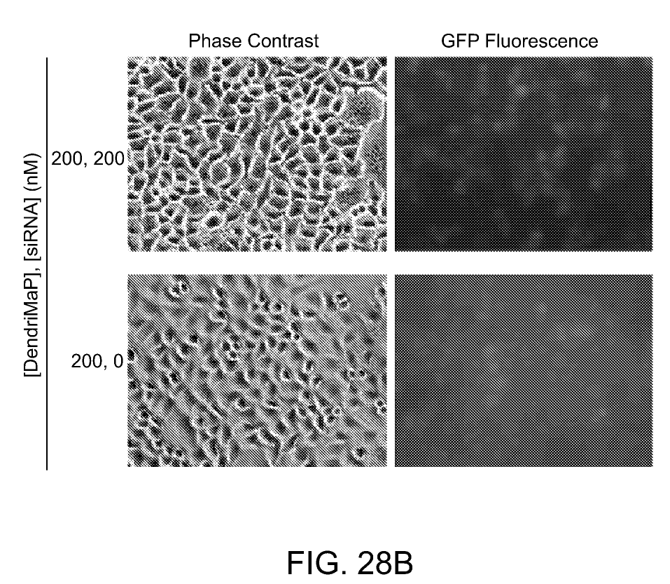

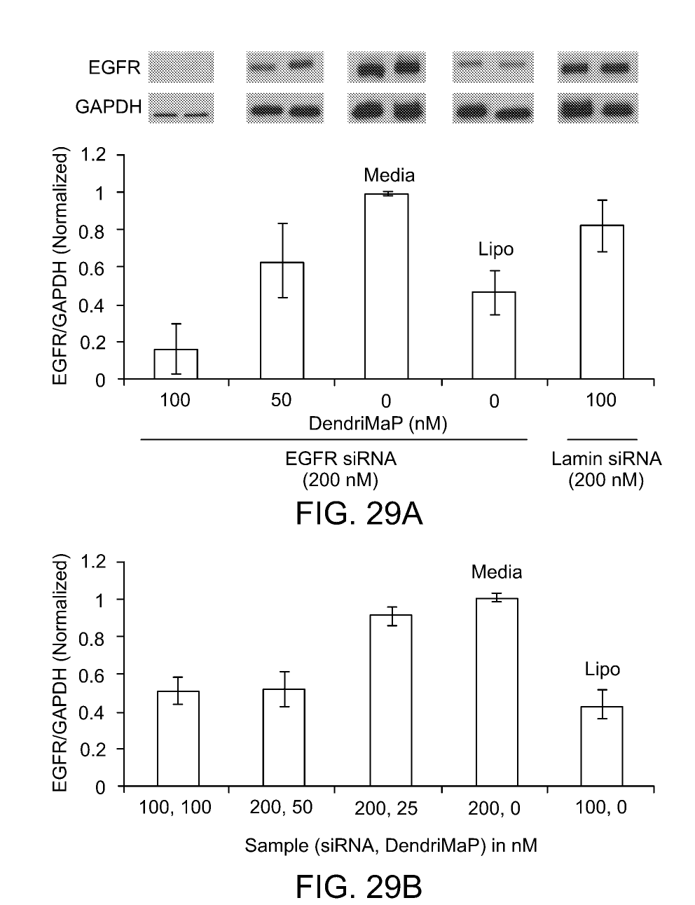

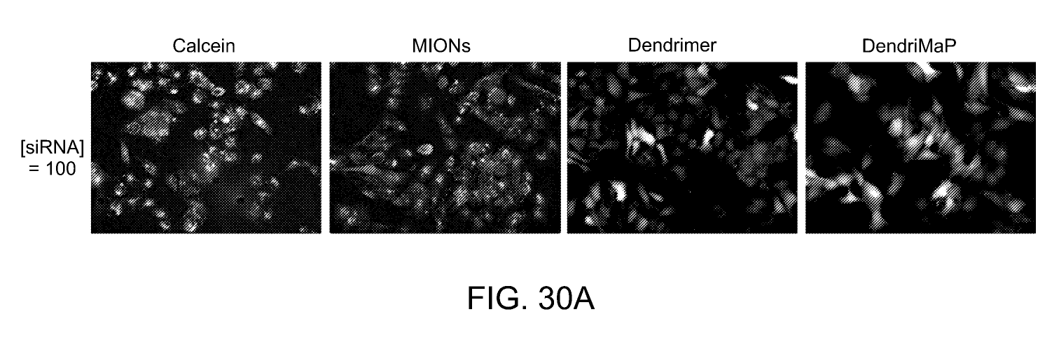

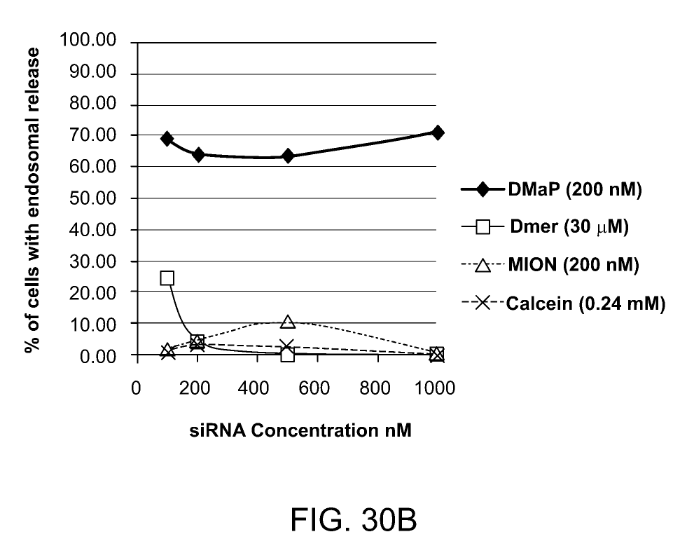

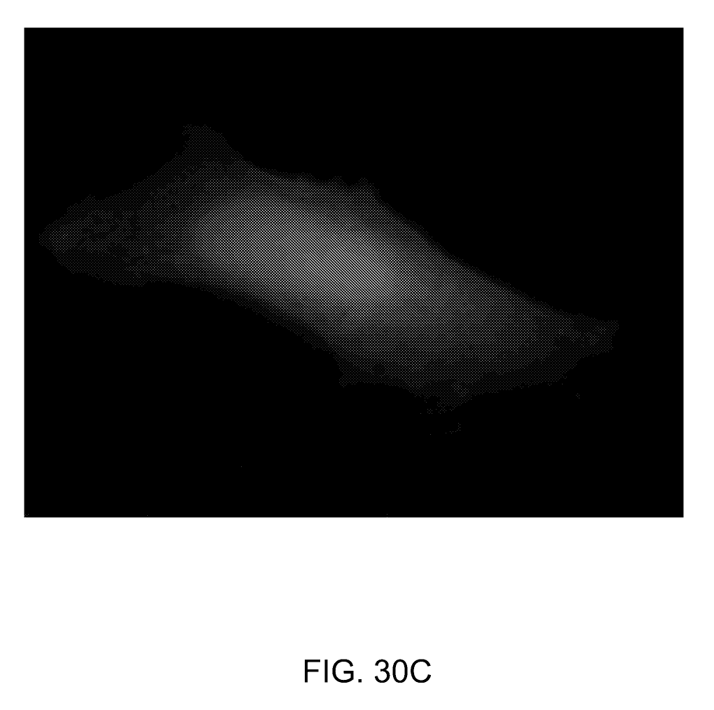

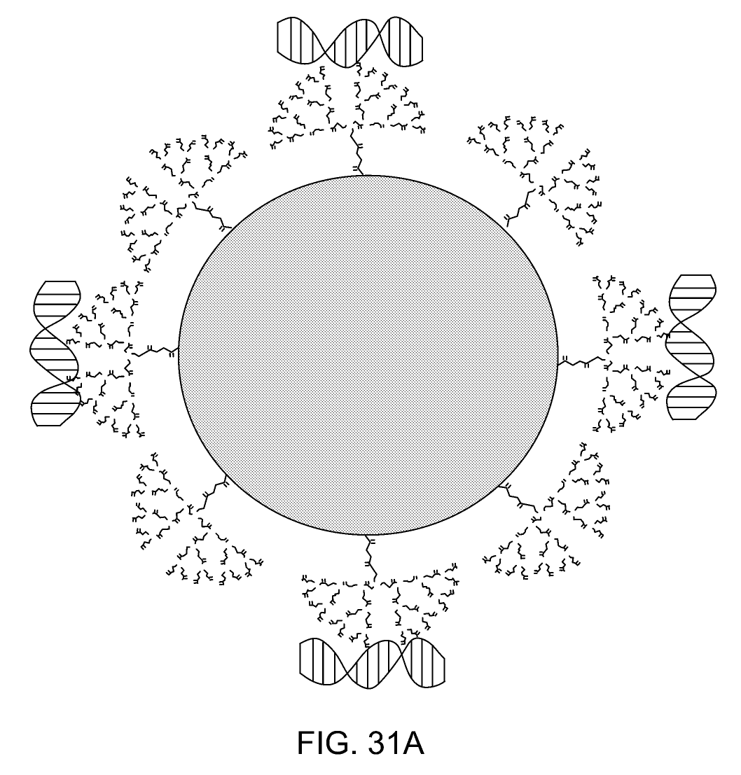

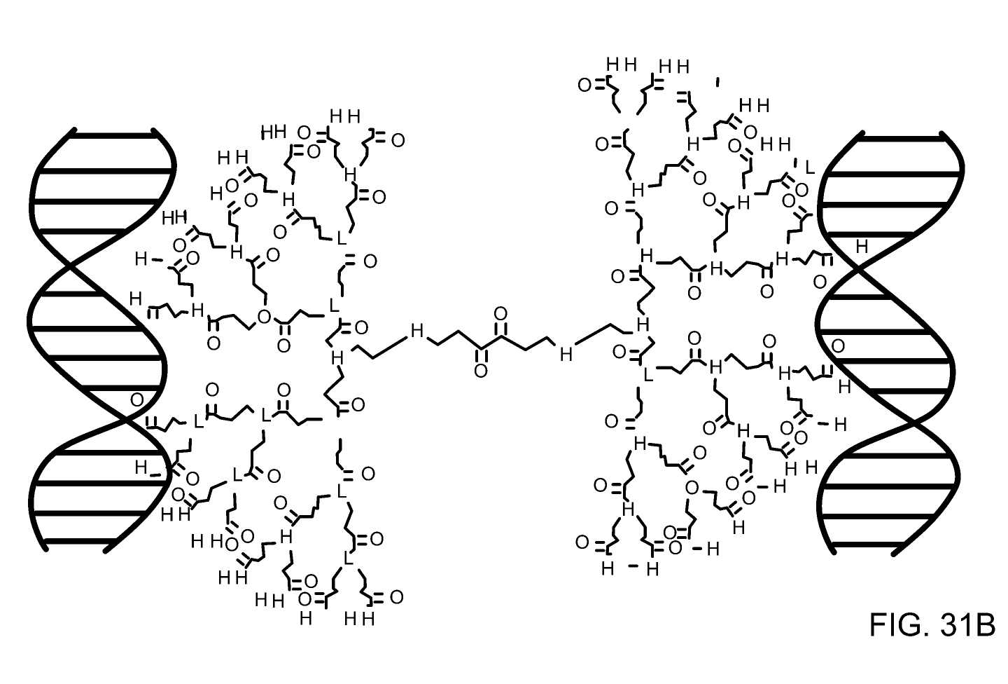

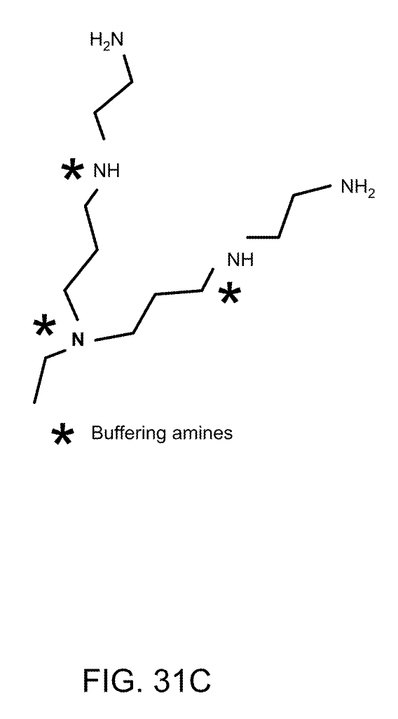

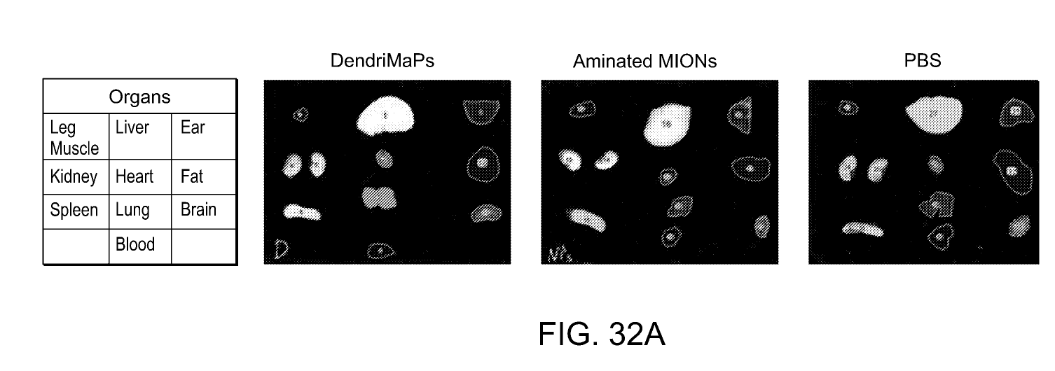

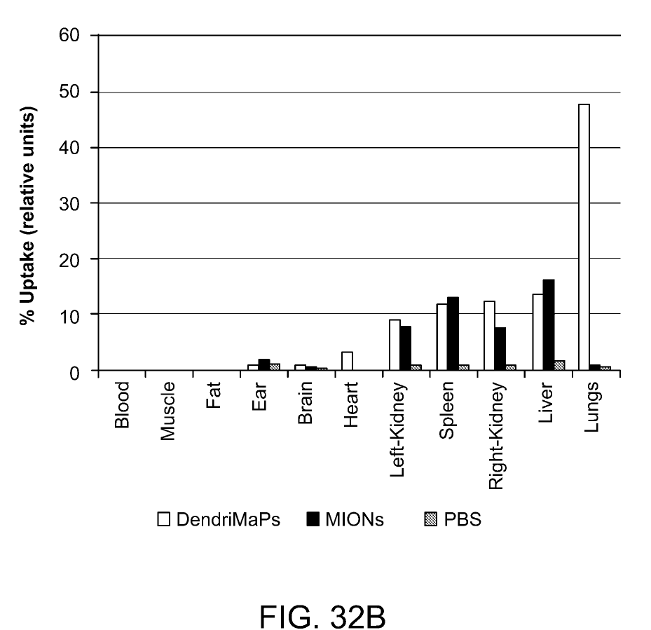

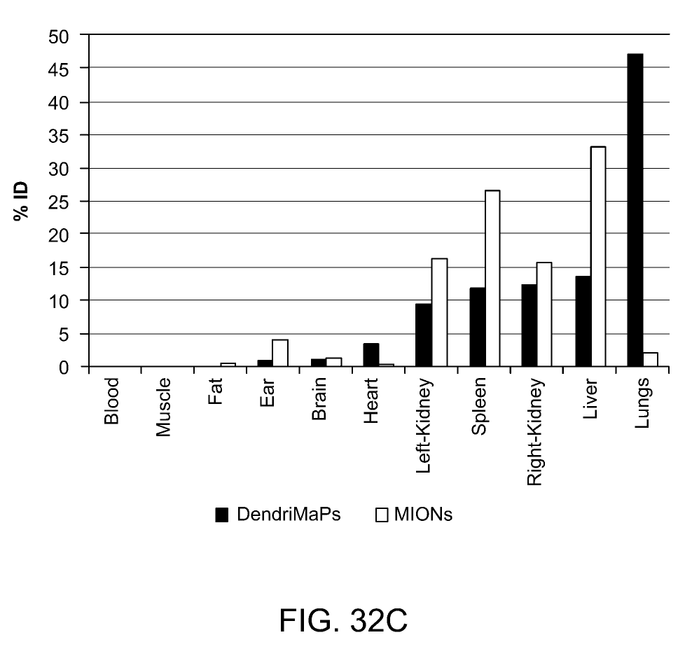

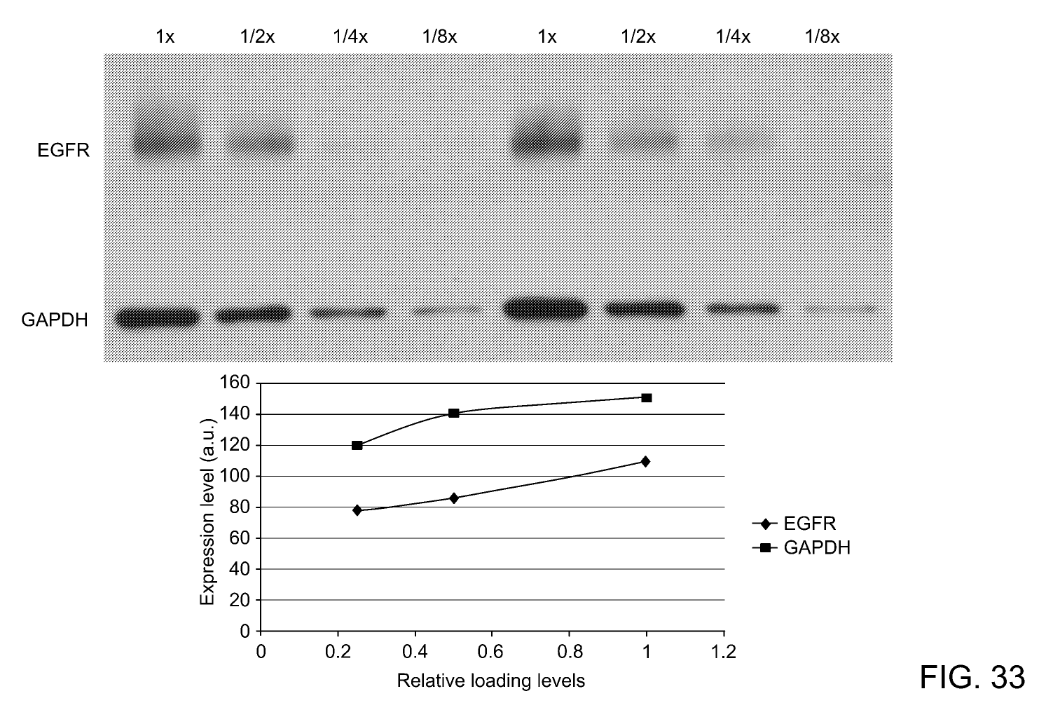

In some embodiments, nanoparticle entities are magnetic iron oxide nanoparticles (“MIONs”) modified (covalently or non-covalently) with branched polymers called Dendrimers or their fractions (e.g. reduced half). As used herein, such entities are referred to as DendriMaPs. In some embodiments, dendrimers can be based on different backbones and chemistries and may be of different generations. Dendrimers used may also be fractured or modified with dye molecules, targeting ligands (e.g., small molecules, nucleic acid sequences, aptamers, peptides, etc.) and other polymers. A DendriMaP may have one or several dendrimers (or their reduced fractions) of one or more type of backbone and from one or more generation. DendriMaPs may have negative, neutral or positive charge and may be of any size. Examples of DendriMaP applications are demonstrated in FIGS. 26-34.

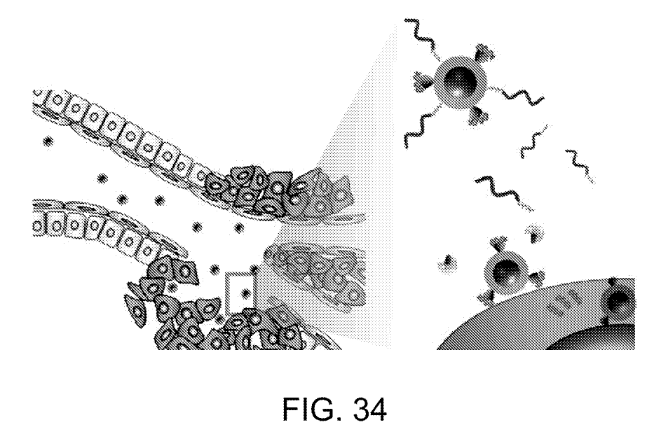

In some embodiments, nanoparticle entities comprising at least one dendrimer may optionally comprise a cloaking entity to help protect the nanoparticle entity from degradation. In some embodiments, such a cloaking entity may stabilize the nanoparticle entity, increase its half-life, and/or increase its circulation time. In some embodiments, a cloaking entity may be polyethylene glycol (PEG), as demonstrated in FIG. 34.

Polysaccharides such as natural and synthetic cyclodextrins and derivatives and modified forms thereof are of use in certain embodiments (see, e.g., U.S. Patent Publication 2003/0157030; and Singh et al., 2002, Biotechnol. Adv., 20:341; both of which are incorporated herein by reference).

In certain embodiments, the transfection reagent forms a complex with one or more nanoparticles and/or agents. Typically the complex will contain a plurality of agents and a plurality of nanoparticles. Components of the complex are physically associated. In some embodiments, the physical association is mediated, for example, by non-covalent interactions such as electrostatic interactions, hydrophobic or hydrophilic interactions, hydrogen bonds, etc., rather than covalent interactions or high affinity specific binding interactions. A complex can be formed when a moiety is encapsulated or entrapped by one or more other moieties. The present invention demonstrates that one or more nanoparticles, modulating entities, agents to be delivered, and transfection reagents can form a complex that is efficiently taken up by mammalian cells and that this uptake can be tracked and monitored by detecting the nanoparticles. In some specific embodiments, the invention encompasses the recognition that an siRNA can retain its gene silencing activity throughout the process of targeted delivery.

Complex formation may take place by a variety of different mechanisms. For example, incubation of a lipid in the presence of agents to be delivered and/or nanoparticles in an aqueous medium may result in formation of a liposome in which the agents to be delivered and/or nanoparticles are encapsulated in an aqueous compartment. Alternatively or additionally, agents to be delivered and/or nanoparticles may be entrapped in, or non-covalently associated with, the surface of the liposome. While not wishing to be bound by any theory, it is hypothesized that certain transfection reagents form a complex with the nanoparticles and/or agents to be delivered via electrostatic interactions. Liposomes formed from a lipid or combination thereof may be coated with a plurality of nanoparticles electrostatically attracted to the liposome surface.

Complexes can be formed, for example, by contacting a transfection reagent and nanoparticles for a period of time sufficient to allow complex formation to occur. The composition is then combined with one or more agents to be delivered and the resulting composition is again maintained for a suitable period of time to allow complex formation to occur. Alternatively or additionally, the transfection reagent and the agents to be delivered can first be allowed to form a complex, following which nanoparticles are combined with the composition. In some embodiments, the transfection reagent, modulating entities, nanoparticles, and agents to be delivered are mixed together and maintained for a suitable time period. Components can be combined by adding one to the other, by adding each of multiple components to a single vessel, etc. Suitable time periods for any of the afore-mentioned steps can be, e.g. several seconds, minutes, or hours (e.g., between 5 min-60 min or 10 min-30 min). Contacting typically takes place in an aqueous medium.

A lipid transfection reagent may contain liposomes. In some embodiments, the liposomes are preformed liposomes. In some embodiments, other structures may form during the contacting. If desired, the physical characteristics of a complex comprising agents to be delivered, modulating entities, nanoparticles, and a transfection reagent can be evaluated using a variety of methods known in the art. For example, the size, charge, and/or polydispersity of the complex can be determined using a Malvern Instruments Zetasizer (Malvern, UK), dynamic light scattering, etc.

Standard transfection protocols can be used to deliver agents and/or nanoparticles to cells. Typically the cells are contacted with the transfection reagent, nanoparticles, and RNA (e.g., as a complex) for time periods ranging from minutes to hours. Protocols can be varied to optimize uptake.

The invention encompasses the use of magnetic forces to enhance uptake of nanoparticles, agents to be delivered, or both, by cells. In some embodiments, a complex comprises a magnetic nanoparticle and an siRNA.

Translocation Entities

In certain embodiments, nanoparticles and/or agents are associated with one or more translocation entities. Translocation entities may be peptides, proteins, glycoproteins, nucleic acids, carbohydrates, lipids, small molecules, etc. Typically, a translocation entity is a peptide. A translocation peptide can be any of a variety of protein domains that are capable of inducing or enhancing translocation of an associated moiety into a eukaryotic cell, e.g., a mammalian cell. For example, presence of these domains within a larger protein enhances transport of the larger protein into cells. These domains are sometimes referred to as protein transduction domains (PTDs) or cell penetrating peptides (CPPs). Translocation peptides include peptides derived from various viruses, DNA binding segments of leucine zipper proteins, synthetic arginine-rich peptides, etc. (see, e.g. Langel, U. (ed.), Cell-Penetrating Peptides: Processes and Applications, CRC Press, Boca Raton, Fla., 2002).

Exemplary translocation peptides that may be used in accordance with the present invention include, but are not limited to, the TAT₄₉₋₅₇ peptide, referred to herein as “TAT peptide” (sequence: RKKRRQRRR (SEQ ID NO: 2)) from the HIV-1 protein (Wadia et al., 2004, Nat. Med., 10:310; and Won et al. 2005, Science, 309:121; both of which are incorporated herein by reference); longer peptides that comprise the TAT peptide; and the peptide RQIKIWFZQRRMKWKK (SEQ ID NO: 3) from the Antennapedia protein.

In some embodiments, translocation-enhancing moieties of use include peptide-like molecules known as peptoid molecular transporters (U.S. Pat. Nos. 6,306,933 and 6,759,387; both of which are incorporated herein by reference). Certain of these molecules contain contiguous, highly basic subunits, particularly subunits containing guanidyl or amidinyl moieties.

Endosome Escape Entities

In some embodiments, an endosome disrupting or fusogenic entity is administered to cells to enhance release of one or more nanoparticles and/or agents to be delivered from endosomes. Examples include fusogenic peptides, chloroquine, various viral components such as the N-terminal portion of the influenza virus HA protein (e.g., the HA2 peptide), adenoviral proteins or portions thereof, etc. (see, e.g., U.S. Pat. No. 6,274,322; incorporated herein by reference). For example, in certain embodiments, the endosome disrupting entity is a peptide comprising the N-terminal 20 amino acids of the influenza HA protein. In some embodiments, the INF-7 peptide, which resembles the NH₂-terminal domain of the influenza virus hemagglutinin HA-2 subunit, is used. In certain embodiments, an endosome escape entity or fusogenic peptide is conjugated to the nanoparticle and/or agent to be delivered.

The membrane-lytic peptide mellitin may be used. In certain embodiments, an endosome disrupting agent is conjugated to an agent, a nanoparticle, or both. In certain embodiments, a polypeptide having a first domain that serves as an endosome disrupting or fusogenic agent and a second domain that serves as a translocation peptide is employed. An agent that enhances release of endosomal contents or escape of an attached moiety from an internal cellular compartment such as an endosome may be referred to as an “endosomal escape agent.”

In some embodiments, nanoparticles and/or agents are sequestered in endosomes for up to 90 minutes before being released. In some embodiments, nanoparticles and/or agents are sequestered in endosomes for up to 6 hours before being released. In some embodiments, nanoparticles and/or agents are sequestered in endosomes for up to 24 hours before being released. In some embodiments, nanoparticles and/or agents are sequestered in endosomes for up to 1 week before being released. In some embodiments, nanoparticles and/or agents are sequestered in endosomes for up to 1 month before being released. In some embodiments, nanoparticles and/or agents are sequestered in endosomes for up to 6 months before being released. In some embodiments, nanoparticles and/or agents remain stable while sequestered in endosomes.

In some embodiments, nanoparticles are associated with one or more entities that cause the nanoparticle to accumulate in the endosomal compartments. This entrapment is followed by endosomal release by peptides or photo-induced release. Endosomal escape can be triggered by heat, light (e.g., UV, visible, near-infrared), electromagnetic radiation, or a chemical. Exemplary chemicals that can trigger endosomal release include, but are not limited to, small molecules (e.g., chloroquine), cationic polymers (e.g. PEI, poly-lysine, protamine), cationic liposomes, peptides (e.g., INF7), proton pump inhibitors, and/or photosensitizers (e.g., porphyrin).

These triggers can affect the endosome compartment directly (e.g. by affecting pore formation or endosomal lysis) and/or can provide energy input to the nanoparticle and/or agents, which is used to disrupt the endosomal membrane. For example, quantum dots can be excited through light or an electromagnetic field, producing an exciton (i.e., an electron-hole pair). Recombination of the electron-hole pair generates stoke-shifted light, but electrons lost to the surroundings can generate free radical species (e.g., oxygen), which can disrupt the endosomal membrane, leading to cytoplasmic delivery of the quantum dot and/or associated agents (see, e.g. Berg et al.; and U.S. Pat. Nos. 6,680,301 and 7,223,600; all of which are incorporated herein by reference).

These trigger entities can be conjugated to nanoparticles chemically or physically to promote endosomal escape of nanoparticles. In case of photosensitizers, light can serve as an additional trigger to activate photosensitizers to generate singlet oxygen which then, induce endosomal escape. In some embodiments, an agent enters the nucleus after endosomal release. In some embodiments, an agent enters the cytosol after endosomal release. In some embodiments, an agent enters the cytosol and then enters the nucleus after endosomal release.

In some embodiments, triggering endosomal escape may promote endosomal release of the agent to be delivered (e.g. an RNAi entity), but not endosomal release of the nanoparticle. For example, endosomal release often results in the nanoparticle being left behind in the endosome, while the agent is released from the endosome and enters the cytosol. While not wishing to be bound by any theory, this phenomenon may be due to endosomal pore-formation, which may dictate size-selective release. Nanoparticles are thought to aggregate in endosomes, leading to even larger nanoparticulate structures. Nanoparticles and/or nanoparticulate aggregates may enter the cytosol on endosome lysis, but not pore formation.

In some embodiments, nanoparticles and/or agents accumulate in endosomes via receptor-mediated endocytosis. Endocytosis is the invagination of the cell membrane and the pinching off of an intracellular, membrane-bound vesicle (endosome). This is a general pathway for internalization of the many ligands (e.g. epidermal growth factor). While not wishing to be bound by any theory, nanoparticles may follow this route when they or an agent to be delivered binds to a cell-surface receptor, triggering internalization and accumulation in endosomes. Thus, in some embodiments, any receptor and/or ligand associated with the nanoparticle and/or any species that the cell recognizes as a ligand (e.g. a ligand mimic) can lead to endosomal accumulation of the nanoparticle and/or agents to be delivered.

It has been thought, that in some cases, internalization can take place via other pathways which do not utilize endosomes (e.g., HIV TAT was thought to work via lipid-raft mediated pinocytosis; Wadia et al., 2004, Nat. Med., 10:310; incorporated herein by reference). However, particles generally end up in the endosomes, even when attached to agents that may initially avoid this pathway (e.g. TAT, F3).

Protective Entities

In certain embodiments, nanoparticles and/or agents are associated with one or more entities that protect an agent to be delivered. In some embodiments, nanoparticles comprising an agent to be delivered may comprise one or more entities that protect against degradation of or damage to the agent. In some embodiments, a biocompatible coating layer may be useful for protecting the agent to be delivered (e.g. to protect an RNAi entity to be delivered from serum nucleases). Suitable protective entities include, but are not limited to, polyethylene glycol (PEG) or a PEG derivative, phospholipid-(PEG), proteins such as bovine serum albumin (BSA), silica, lipids, carbohydrates such as dextran, etc.

In some embodiments, protective entities may be associated with the agent. Such association may be covalent or non-covalent.

In some embodiments, protective entities may coat the nanoparticle. Such coating layers may be applied or assembled in a variety of ways such as by dipping, using a layer-by-layer technique, by self-assembly, etc. Self-assembly refers to a process of spontaneous assembly of a higher order structure that relies on the natural attraction of the components of the higher order structure (e.g., molecules) for each other. It typically occurs through random movements of the molecules and formation of bonds based on size, shape, composition or chemical properties.

In some embodiments, an agent can be modified in any way which protects it from degradation. In some embodiments, an agent can be covalently or non-covalently modified in order to protect the agent from degradation. For example, the agent can be coated with PEG or another protective agent. Alternatively or additionally, a nucleic acid agent can include non-standard nucleotides, as described herein, which protect the nucleic acid from endonuclease activity.

Entities that Alter Activity of an Agent

In certain embodiments, nanoparticles and/or agents are associated with one or more entities that alter the activity of an agent. In some embodiments, such entities may enhance the activity of an agent to be delivered. In some embodiments, such entities may include cationic reagents that enhance the activity of an agent to be delivered. Cationic polymers such as PEI, poly-lysine, and protamine are known to be additives to enhance activities of polynucleotides in cells.

Entities that Mediate Controlled Release of an Agent

In certain embodiments, nanoparticles and/or agents are associated with one or more entities that mediate controlled release of an agent. In some embodiments, an agent and targeting peptide are conjugated to nanoparticles via protease-cleavable peptides. Cleavage will occur the sites where corresponding proteases are present. Proteases such as matrix metalloproteases (MMPs) are upregulated in many types of tumors. Therefore, agents to be delivered that are conjugated to nanoparticle entities via protease-cleavable bonds are released from nanoparticles when nanoparticles reach tumor sites in vivo.

In general, agents (e.g. siRNAs, drugs, etc.) can be associated with nanoparticles using a protease-sensitive sequence. Serine proteases or MMPs have specific peptide sequences that they typically recognize and cleave. In some embodiments, one end of the target peptide is conjugated to the particle (covalently or non-covalently), with the other end conjugated to the cargo (covalently or non-covalently). In some embodiments, heterobifunctional crosslinkers (e.g. sulfo-SPDP or sulfo-SMCC) are used to conjugate an amino group on one species (e.g. nanoparticle) to a thiol group on the other (e.g. cysteine residue on the peptide). In some embodiments, a target peptide/nanoparticle conjugate can be linked to an agent with an additional conjugation step (e.g. a lysine residue on the peptide can be reacted with sulfo-SMCC to form a maleimide, which in turn can react with a thiol group added to the agent). Appropriate peptide sequences can be produced synthetically or expressed in a cell culture system. Purification (e.g. HPLC) is typically performed to ensure that only the sequence of interest is conjugated between the nanoparticle and agent.

Exemplary peptide sequences and proteases that target these sequences are presented in Table 1 (adapted from Funovics et al., 2003, Anal. Bioanal. Chem., 377:956; and Harris et al., 2006, Angew. Chem. Int. Ed., 45:3161; both of which are incorporated herein by reference):

TABLE 1
Peptide Sequences Cleavable by Proteases

| | | |
|---|---|---|
| Target protease | Disease | Substrate Peptide |

| | | |
|---|---|---|
| Cathepsin B | Cancer | K•K |
| | | (SEQ ID NO: 4) |
| PSA | Prostate cancer | HSSKLQ• |
| | | (SEQ ID NO: 5) |
| Cathepsin D | Breast cancer | PICF•F |
| | | (SEQ ID NO: 6) |
| MMP-2 | Metastases | GPLG•VRG |
| | | (SEQ ID NO: 7) |
| HIV protease | HIV | GVSQNY•PIVG |
| | | (SEQ ID NO: 8) |
| HSV protease | HSV | LVLA•SSSFGY |
| | | (SEQ ID NO: 9) |
| Caspase-3 | Apoptosis | DEVD• |
| | | (SEQ ID NO: 10) |
| Caspase-1 (ICE) | Apoptosis | WEHD• |
| | | (SEQ ID NO: 11) |
| Thrombin | Cardiovascular | F(Pip*)R•S |

*Pip: pipeloic acid
•indicates cleavage site.

In some embodiments, other proteases that could serve as target proteases according to the present invention include, but are not limited to, any matrix metalloprotease (e.g. MMP-1, MMP-7, MMP-9, MMP-13, etc.), Caspase-2, NFκB, Cathespin S, Cathespin K, etc.

In some embodiments, other proteases that could serve as target proteases according to the present invention include, but are not limited to, any matrix metalloprotease (e.g. MMP-1, MMP-7, MMP-9, MMP-13, etc.), Caspase-2, NFκB, Cathespin S, Cathespin K, etc.

When a nanoparticle and/or agent is introduced into a region of high protease expression (e.g. targeted to tumor interstitium where a high concentration of MMPs are present), extracellular cleavage leads to separation of the nanoparticle and agent. Whereas, without the proteases present, the agent remains attached.

In some embodiments, nanoparticles and/or agents are associated with one or more modulating entities (e.g. cell-penetrating peptides, translocation entities such as dendrimers, targeting entities, etc.) and subsequently associated with polyethylene glycol (PEG), which can serve to cloak the nanoparticle and modulating entities. In some embodiments, PEG is covalently associated with the nanoparticle and/or modulating entities. In some embodiments, PEG is covalently linked to the nanoparticle and/or modulating entities by a linker (e.g. a peptide linker). In some embodiments, a peptide linker is a recognition signal for cleavage by a protease (including, but not limited to, the proteins and recognition sequences described above). In some embodiments, the protease is one that is expressed in target cells (e.g. tumor cells). In certain embodiments, the protease is one that is expressed at higher levels in tumor cells relative to non-tumor cells. When the nanoparticle associated with PEG and one or more modulating entities reaches a tumor cell, protease cleaves the peptide at the recognition site, thereby unmasking the modulating entity and allowing the nanoparticle associated with modulating entities to enter the cell. In certain embodiments, the nanoparticle is further associated with an agent to be delivered, and this agent is delivered upon uncloaking and cellular entry. An example of protease-triggered unveiling of bioactive nanoparticles is described in Example 13.

In certain embodiments, a degradable (e.g. hydrolytically degradable) polymeric particle may be cloaked via a coating (e.g. PEG), as described herein. Example 14 describes how a one exemplary polymer, C32, which is normally unstable at physiological pH, can surprisingly be made more stable by associating the particle with a PEG coating. This increased stability leads to increased half-life and increased circulation times.

### Agents to Be Delivered

According to the present invention, any agents, including, for example, therapeutic, diagnostic, and/or prophylactic agents may be delivered. Exemplary agents to be delivered in accordance with the present invention include, but are not limited to, small molecules, organometallic compounds, nucleic acids, proteins (including multimeric proteins, protein complexes, etc.), peptides, lipids, carbohydrates, hormones, metals, radioactive elements and compounds, drugs, vaccines, immunological agents, etc., and/or combinations thereof. In some embodiments, the agents to be delivered are functional RNAs (e.g. siRNAs and shRNAs, tRNAs, ribozymes, RNAs used for triple helix formation, etc.).

Functional RNAs and their Activities

In certain embodiments, a nanoparticle is used to deliver one or more functional RNAs to a specific location such as a tissue, cell, or subcellular locale. In some such embodiments, the RNA is an RNA that does not code for a protein but instead belongs to a class of RNA molecules whose members characteristically possess one or more different functions or activities within a cell. Such RNAs are referred to herein as “functional RNAs.”

It will be appreciated that the relative activities of functional RNA molecules having different sequences may differ and may depend at least in part on the particular cell type in which the RNA is present. Thus the term “functional RNA” is used herein to refer to a class of RNA molecule and is not intended to imply that all members of the class will in fact display the activity characteristic of that class under any particular set of conditions. While the scope of RNAs whose cellular uptake and/or activity can be achieved is in no way limited, the invention finds particular use for delivering short RNAi agents and tRNAs.

As mentioned above, RNAi is an evolutionarily conserved process in which presence of an at least partly double-stranded RNA molecule in a eukaryotic cell leads to sequence-specific inhibition of gene expression. RNAi was originally described as a phenomenon in which the introduction of long dsRNA (typically hundreds of nucleotides) into a cell results in degradation of mRNA containing a region complementary to one strand of the dsRNA (U.S. Pat. No. 6,506,559; and Fire et al., 1998, Nature, 391:806; both of which are incorporated herein by reference). Subsequent studies in Drosophila showed that long dsRNAs are processed by an intracellular RNase III-like enzyme called Dicer into smaller dsRNAs primarily comprised of two approximately 21 nucleotide (nt) strands that form a 19 base pair duplex with 2 nt 3′ overhangs at each end and 5′-phosphate and 3′-hydroxyl groups (see, e.g. PCT Publication WO 01/75164; U.S. Patent Publications 2002/0086356 and 2003/0108923; Zamore et al., 2000, Cell, 101:25; and Elbashir et al., 2001, Genes Dev., 15:188; all of which are incorporated herein by reference).

Short dsRNAs having structures such as this, referred to as siRNAs, silence expression of genes that include a region that is substantially complementary to one of the two strands. This strand is referred to as the “antisense” or “guide” strand, with the other strand often being referred to as the “sense” strand. The siRNA is incorporated into a ribonucleoprotein complex termed the RNA-induced silencing complex (RISC) that contains member(s) of the Argonaute protein family. Following association of the siRNA with RISC, a helicase activity unwinds the duplex, allowing an alternative duplex to form the guide strand and a target mRNA containing a portion substantially complementary to the guide strand. An endonuclease activity associated with the Argonaute protein(s) present in RISC is responsible for “slicing” the target mRNA, which is then further degraded by cellular machinery.

Considerable progress towards the practical application of RNAi was achieved with the discovery that exogenous introduction of siRNAs into mammalian cells can effectively reduce the expression of target genes in a sequence-specific manner via the mechanism described above. A typical siRNA structure includes a 19 nucleotide double-stranded portion, comprising a guide strand and an antisense strand. Each strand has a 2 nt 3′ overhang. Typically the guide strand of the siRNA is perfectly complementary to its target gene and mRNA transcript over at least 17-19 contiguous nucleotides, and typically the two strands of the siRNA are perfectly complementary to each other over the duplex portion. However, as will be appreciated by one of ordinary skill in the art, perfect complementarity is not required. Instead, one or more mismatches in the duplex formed by the guide strand and the target mRNA is often tolerated, particularly at certain positions, without reducing the silencing activity below useful levels. For example, there may be 1, 2, 3, or even more mismatches between the target mRNA and the guide strand (disregarding the overhangs). Thus, as used herein, two nucleic acid portions such as a guide strand (disregarding overhangs) and a portion of a target mRNA that are “substantially complementary” may be perfectly complementary (i.e., they hybridize to one another to form a duplex in which each nucleotide is a member of a complementary base pair) or they may have a lesser degree of complementarity sufficient for hybridization to occur. One of ordinary skill in the art will appreciate that the two strands of the siRNA duplex need not be perfectly complementary. Typically at least 80%, at least 90%, or more of the nucleotides in the guide strand of an effective siRNA are complementary to the target mRNA over at least about 19 contiguous nucleotides. The effect of mismatches on silencing efficacy and the locations at which mismatches may most readily be tolerated are areas of active study (see, e.g. Reynolds et al., 2004, Nat. Biotechnol., 22:326; incorporated herein by reference).

It will be appreciated that molecules having the appropriate structure and degree of complementarity to a target gene will exhibit a range of different silencing efficiencies. A variety of additional design criteria have been developed to assist in the selection of effective siRNA sequences. Numerous software programs that can be used to choose siRNA sequences that are predicted to be particularly effective to silence a target gene of choice are available (see, e.g., Yuan et al., 2004, Nuc. Acid. Res., 32:W130; and Santoyo et al., 2005, Bioinformatics, 21:1376; both of which are incorporated herein by reference).

As will be appreciated by one of ordinary skill in the art, RNAi may be effectively mediated by RNA molecules having a variety of structures that differ in one or more respects from that described above. For example, the length of the duplex can be varied (e.g., from about 17-29 nucleotides); the overhangs need not be present and, if present, their length and the identity of the nucleotides in the overhangs can vary (though most commonly symmetric dTdT overhangs are employed in synthetic siRNAs).

Additional structures, referred to as short hairpin RNAs (shRNAs), are capable of mediating RNA interference. An shRNA is a single RNA strand that contains two complementary regions that hybridize to one another to form a double-stranded “stem,” with the two complementary regions being connected by a single-stranded loop. shRNAs are processed intracellularly by Dicer to form an siRNA structure containing a guide strand and an antisense strand. While shRNAs can be delivered exogenously to cells, more typically intracellular synthesis of shRNA is achieved by introducing a plasmid or vector containing a promoter operably linked to a template for transcription of the shRNA into the cell, e.g., to create a stable cell line or transgenic organism.

While sequence-specific cleavage of target mRNA is currently the most widely used means of achieving gene silencing by exogenous delivery of short RNAi agents to cells, additional mechanisms of sequence-specific silencing mediated by short RNA species are known. For example, post-transcriptional gene silencing mediated by small RNA molecules can occur by mechanisms involving translational repression. Certain endogenously expressed RNA molecules form hairpin structures containing an imperfect duplex portion in which the duplex is interrupted by one or more mismatches and/or bulges. These hairpin structures are processed intracellularly to yield single-stranded RNA species referred to as known as microRNAs (miRNAs), which mediate translational repression of a target transcript to which they hybridize with less than perfect complementarity. siRNA-like molecules designed to mimic the structure of miRNA precursors have been shown to result in translational repression of target genes when administered to mammalian cells.

Thus the exact mechanism by which a short RNAi agent inhibits gene expression appears to depend, at least in part, on the structure of the duplex portion of the RNAi agent and/or the structure of the hybrid formed by one strand of the RNAi agent and a target transcript. RNAi mechanisms and the structure of various RNA molecules known to mediate RNAi, e.g. siRNA, shRNA, miRNA and their precursors, have been extensively reviewed (see, e.g. Dykxhhorn et al., 2003, Nat. Rev. Mol. Cell. Biol., 4:457; Hannon and Rossi, 2004, Nature, 431:3761; and Meister and Tuschl, 2004, Nature, 431:343; all of which are incorporated herein by reference). It is to be expected that future developments will reveal additional mechanisms by which RNAi may be achieved and will reveal additional effective short RNAi agents. Any currently known or subsequently discovered short RNAi agents are within the scope of the present invention.

A short RNAi agent that is delivered by methods in accordance with the present invention and/or is present in a composition in accordance with the invention may be designed to silence any eukaryotic gene. The gene can be a mammalian gene, e.g., a human gene. The gene can be a wild type gene, a mutant gene, an allele of a polymorphic gene, etc. The gene can be disease-associated, e.g., a gene whose over-expression, under-expression, or mutation is associated with or contributes to development or progression of a disease. For example, the gene can be oncogene. The gene can encode a receptor or putative receptor for an infectious agent such as a virus (see, e.g., Dykxhhorn et al., 2003, Nat. Rev. Mol. Cell. Biol., 4:457; incorporated herein by reference).

In some embodiments, tRNAs are functional RNA molecules whose delivery to eukaryotic cells can be monitored using the compositions and methods in accordance with the invention. The structure and role of tRNAs in protein synthesis is well known (Soll and Rajbhandary, (eds.) tRNA: Structure, Biosynthesis, and Function, ASM Press, 1995). The cloverleaf shape of tRNAs includes several double-stranded “stems” that arise as a result of formation of intramolecular base pairs between complementary regions of the single tRNA strand. There is considerable interest in the synthesis of polypeptides that incorporate unnatural amino acids such as amino acid analogs or labeled amino acids at particular positions within the polypeptide chain (see, e.g., Köhrer and RajBhandary, “Proteins carrying one or more unnatural amino acids,” Chapter 33, In Ibba et al., (eds.), Aminoacyl-tRNA Synthetases, Landes Bioscience, 2004). One approach to synthesizing such polypeptides is to deliver a suppressor tRNA that is aminoacylated with an unnatural amino acid to a cell that expresses an mRNA that encodes the desired polypeptide but includes a nonsense codon at one or more positions. The nonsense codon is recognized by the suppressor tRNA, resulting in incorporation of the unnatural amino acid into a polypeptide encoded by the mRNA (Kohrer et al., 2001, Proc. Natl. Acad. Sci., USA, 98:14310; and Kohrer et al., 2004, Nuc. Acid. Res., 32:6200; both of which are incorporated herein by reference). However, as in the case of siRNA delivery, existing methods of delivering tRNAs to cells result in variable levels of delivery, complicating efforts to analyze such proteins and their effects on cells.

The invention contemplates the delivery of tRNAs, e.g. suppressor tRNAs, and optically or magnetically detectable nanoparticles to eukaryotic cells in order to achieve the synthesis of proteins that incorporate an unnatural amino acid with which the tRNA is aminoacylated. The analysis of proteins that incorporate one or more unnatural amino acids has a wide variety of applications. For example, incorporation of amino acids modified with detectable (e.g., fluorescent) moieties can allow the study of protein trafficking, secretion, etc., with minimal disturbance to the native protein structure. Alternatively or additionally, incorporation of reactive moieties (e.g., photoactivatable and/or cross-linkable groups) can be used to identify protein interaction partners and/or to define three-dimensional structural motifs. Incorporation of phosphorylated amino acids such as phosphotyrosine, phosphothreonine, or phosphoserine, or analogs thereof, into proteins can be used to study cell signaling pathways and requirements.

In some embodiments, the functional RNA is a ribozyme. A ribozyme is designed to catalytically cleave target mRNA transcripts may be used to prevent translation of a target mRNA and/or expression of a target (see, e.g. PCT publication WO 90/11364; and Sarver et al., 1990, Science 247:1222; both of which are incorporated herein by reference).

In some embodiments, endogenous target gene expression may be reduced by targeting deoxyribonucleotide sequences complementary to the regulatory region of the target gene (i.e., the target gene's promoter and/or enhancers) to form triple helical structures that prevent transcription of the target gene in target muscle cells in the body (see generally, Helene, 1991, Anticancer Drug Des. 6:569; Helene et al., 1992, Ann, N. Y Acad. Sci. 660:27; and Maher, 1992, Bioassays 14:807; all of which are incorporated herein by reference).

RNAs such as RNAi agents, tRNAs, ribozymes, etc., for delivery to eukaryotic cells may be prepared according to any available technique including, but not limited to chemical synthesis, enzymatic synthesis, enzymatic or chemical cleavage of a longer precursor, etc. Methods of synthesizing RNA molecules are known in the art (see, e.g. Gait, M. J. (ed.) Oligonucleotide synthesis: a practical approach, Oxford [Oxfordshire], Washington, D.C.: IRL Press, 1984; and Herdewijn, P. (ed.) Oligonucleotide synthesis: methods and applications, Methods in molecular biology, v. 288 (Clifton, N.J.) Totowa, N.J.: Humana Press, 2005). Short RNAi agents such as siRNAs are commercially available from a number of different suppliers. Pre-tested siRNAs targeted to a wide variety of different genes are available, e.g., from Ambion (Austin, Tex.), Dharmacon (Lafayette, Colo.), Sigma-Aldrich (St. Louis, Mo.).

When siRNAs are synthesized in vitro the two strands are typically allowed to hybridize before contacting them with cells. It will be appreciated that the resulting siRNA composition need not consist entirely of double-stranded (hybridized) molecules. For example, an RNAi agent commonly includes a small proportion of single-stranded RNA. Generally, at least approximately 50%, at least approximately 90%, at least approximately 95%, or even at least approximately 99%-100% of the RNAs in an siRNA composition are double-stranded when contacted with cells. However, a composition containing a lower proportion of dsRNA may be used, provided that it contains sufficient dsRNA to be effective.

It will be appreciated by those of ordinary skill in the art that synthetic RNAs such as RNAi agents may comprise nucleotides entirely of the types found in naturally occurring nucleic acids, or may instead include one or more nucleotide analogs or have a structure that otherwise differs from that of a naturally occurring nucleic acid. U.S. Pat. Nos. 6,403,779; 6,399,754; 6,225,460; 6,127,533; 6,031,086; 6,005,087; 5,977,089; and references therein (incorporated herein by reference) disclose a wide variety of specific nucleotide analogs and modifications that may be used in a functional RNA. See Crooke, S. (ed.) Antisense Drug Technology: Principles, Strategies, and Applications (1^{st} ed), Marcel Dekker; ISBN: 0824705661; 1st edition (2001) and references therein. For example, 2′-modifications include halo, alkoxy and allyloxy groups. In some embodiments, the 2′-OH group is replaced by a group selected from H, OR, R, halo, SH, SR₁, NH₂, NHR, NR₂ or CN, wherein R is C₁-C₆ alkyl, alkenyl or alkynyl and halo is F, Cl, Br or I. Examples of modified linkages include phosphorothioate and 5′-N-phosphoramidite linkages.

Nucleic acids containing a variety of different nucleotide analogs, modified backbones, or non-naturally occurring internucleoside linkages can effectively mediate RNAi provided that they have contain a guide strand with a nucleobase sequence that is sufficiently complementary to the target gene. In some cases, RNAi agents containing such modifications display improved properties relative to nucleic acids consisting only of naturally occurring nucleotides. For example, the structure of an siRNA may be stabilized by including nucleotide analogs at the 3′ end of one or both strands order to reduce digestion, e.g. by exonucleases.

Modified nucleic acids need not be uniformly modified along the entire length of the molecule. Different nucleotide modifications and/or backbone structures may exist at various positions in the nucleic acid. One of ordinary skill in the art will appreciate that the nucleotide analogs or other modification(s) may be located at any position(s) of an RNAi agent such that the target-specific silencing activity is not substantially affected. The modified region may be at the 5′-end and/or the 3′-end of one or both strands. For example, modified siRNAs in which approximately 1 to approximately 5 residues at the 5′ and/or 3′ end of either of both strands are nucleotide analogs and/or have a backbone modification have been employed. The modification may be a 5′ or 3′ terminal modification. One or both nucleic acid strands of an active RNAi agent may comprise at least 50% unmodified RNA, at least 80% modified RNA, at least 90% unmodified RNA, or 100% unmodified RNA. In certain embodiments, one or more of the nucleic acids in an RNAi agent comprises 100% unmodified RNA within the portion of the guide strand that participates in duplex formation with a target nucleic acid.

RNAi agents may, for example, contain a modification to a sugar, nucleoside, or internucleoside linkage such as those described in U.S. Patent Publications 2003/0175950, 2004/0192626, 2004/0092470, 2005/0020525, and 2005/0032733 (all of which are incorporated herein by reference). Studies describing the effect of a variety of different siRNA modifications have been reviewed (see Manoharan, 2004, Curr. Opin. Chem. Biol., 8:570; incorporated herein by reference). The present invention encompasses the use of an RNAi agent having any one or more of the modification described therein. For example, a number of terminal conjugates, e.g., lipids such as cholesterol, lithocholic acid, aluric acid, or long alkyl branched chains have been reported to improve cellular uptake. Analogs and modifications may be tested using, e.g. using assays such as Western blots, immunofluorescence, or any appropriate assay known in the art, in order to select those that effectively reduce expression of target genes and/or result in improved stability, uptake, etc.

Small Molecules

In some embodiments, the agent to be delivered is a small molecule and/or organic compound with pharmaceutical activity. In some embodiments, the agent is a clinically-used drug. In some embodiments, the drug is an antibiotic, anti-viral agent, anesthetic, anticoagulant, anti-cancer agent, inhibitor of an enzyme, steroidal agent, anti-inflammatory agent, anti-neoplastic agent, antigen, vaccine, antibody, decongestant, antihypertensive, sedative, birth control agent, progestational agent, anti-cholinergic, analgesic, anti-depressant, anti-psychotic, β-adrenergic blocking agent, diuretic, cardiovascular active agent, vasoactive agent, non-steroidal anti-inflammatory agent, etc.

In some embodiments, the agent to be delivered may be a mixture of pharmaceutically active agents. For example, a local anesthetic may be delivered in combination with an anti-inflammatory agent such as a steroid. Local anesthetics may also be administered with vasoactive agents such as epinephrine. To give but another example, an antibiotic may be combined with an inhibitor of the enzyme commonly produced by bacteria to inactivate the antibiotic (e.g. penicillin and clavulanic acid).

Proteins

In some embodiments, the agent to be delivered may be a protein or peptide. In certain embodiments, peptides range from about 5 to about 40, about 10 to about 35, about 15 to about 30, or about 20 to about 25 amino acids in size. Peptides from panels of peptides comprising random sequences and/or sequences which have been varied consistently to provide a maximally diverse panel of peptides may be used.

The terms “polypeptide” and “peptide” are used interchangeably herein, with “peptide” typically referring to a polypeptide having a length of less than about 50 amino acids. Polypeptides may contain L-amino acids, D-amino acids, or both and may contain any of a variety of amino acid modifications or analogs known in the art. Useful modifications include, e.g., terminal acetylation, amidation, etc.

In some embodiments, the agent to be delivered may be an antibody. In some embodiments, antibodies may include, but are not limited to, polyclonal, monoclonal, chimeric (i.e. “humanized”), single chain (recombinant) antibodies. In some embodiments, antibodies may have reduced effector functions and/or bispecific molecules. In some embodiments, antibodies may include Fab fragments and/or fragments produced by a Fab expression library.

Carbohydrates

In some embodiments, the agent to be delivered is a carbohydrate. The carbohydrate may be natural or synthetic. The carbohydrate may also be a derivatized natural carbohydrate. In certain embodiments, the carbohydrate may be a simple or complex sugar. In certain embodiments, the carbohydrate is a monosaccharide, including but not limited to glucose, fructose, galactose, and ribose. In certain embodiments, the carbohydrate is a disaccharide, including but not limited to lactose, sucrose, maltose, trehalose, and cellobiose. In certain embodiments, the carbohydrate is a polysaccharide, including but not limited to cellulose, microcrystalline cellulose, hydroxypropyl methylcellulose (HPMC), methylcellulose (MC), dextrose, dextran, glycogen, xanthan gum, gellan gum, starch, and pullulan. In certain embodiments, the carbohydrate is a sugar alcohol, including but not limited to mannitol, sorbitol, xylitol, erythritol, maltitol, and lactitol.

Lipids

In some embodiments, the agent to be delivered is a lipid. Exemplary lipids that may be used in accordance with the present invention include, but are not limited to, oils, fatty acids, saturated fatty acid, unsaturated fatty acids, essential fatty acids, cis fatty acids, trans fatty acids, glycerides, monoglycerides, diglycerides, triglycerides, hormones, steroids (e.g., cholesterol, bile acids), vitamins (e.g. vitamin E), phospholipids, sphingolipids, and lipoproteins.

In some embodiments, the lipid may comprise one or more fatty acid groups or salts thereof. In some embodiments, the fatty acid group may comprise digestible, long chain (e.g., C₈-C₅₀), substituted or unsubstituted hydrocarbons. In some embodiments, the fatty acid group may be a C₁₀-C₂₀ fatty acid or salt thereof. In some embodiments, the fatty acid group may be a C₁₅-C₂₀ fatty acid or salt thereof. In some embodiments, the fatty acid group may be a C₁₅-C₂₅ fatty acid or salt thereof. In some embodiments, the fatty acid group may be unsaturated. In some embodiments, the fatty acid group may be monounsaturated. In some embodiments, the fatty acid group may be polyunsaturated. In some embodiments, a double bond of an unsaturated fatty acid group may be in the cis conformation. In some embodiments, a double bond of an unsaturated fatty acid may be in the trans conformation.

In some embodiments, the fatty acid group may be one or more of butyric, caproic, caprylic, capric, lauric, myristic, palmitic, stearic, arachidic, behenic, or lignoceric acid. In some embodiments, the fatty acid group may be one or more of palmitoleic, oleic, vaccenic, linoleic, alpha-linolenic, gamma-linoleic, arachidonic, gadoleic, arachidonic, eicosapentaenoic, docosahexaenoic, or erucic acid.

Diagnostic Agents

In some embodiments, the agent to be delivered is a diagnostic agent. In some embodiments, diagnostic agents include gases; commercially available imaging agents used in positron emissions tomography (PET), computer assisted tomography (CAT), single photon emission computerized tomography, x-ray, fluoroscopy, and magnetic resonance imaging (MRI); and contrast agents. Examples of suitable materials for use as contrast agents in MRI include gadolinium chelates, as well as iron, magnesium, manganese, copper, and chromium. Examples of materials useful for CAT and x-ray imaging include iodine-based materials.

Prophylactic Agents

In some embodiments, the agent to be delivered is a prophylactic agent. In some embodiments, prophylactic agents include vaccines. Vaccines may comprise isolated proteins or peptides, inactivated organisms and viruses, dead organisms and virus, genetically altered organisms or viruses, and cell extracts. Prophylactic agents may be combined with interleukins, interferon, cytokines, and adjuvants such as cholera toxin, alum, Freund's adjuvant, etc. Prophylactic agents may include antigens of such bacterial organisms as Streptococccus pnuemoniae, Haemophilus influenzae, Staphylococcus aureus, Streptococcus pyrogenes, Corynebacterium diphtheriae, Listeria monocytogenes, Bacillus anthracis, Clostridium tetani, Clostridium botulinum, Clostridium perfringens, Neisseria meningitidis, Neisseria gonorrhoeae, Streptococcus mutans, Pseudomonas aeruginosa, Salmonella typhi, Haemophilus parainfluenzae, Bordetella pertussis, Francisella tularensis, Yersinia pestis, Vibrio cholerae, Legionella pneumophila, Mycobacterium tuberculosis, Mycobacterium leprae, Treponema pallidum, Leptospirosis interrogans, Borrelia burgdorferi, Camphylobacter jejuni, and the like; antigens of such viruses as smallpox, influenza A and B, respiratory syncytial virus, parainfluenza, measles, HIV, varicella-zoster, herpes simplex 1 and 2, cytomegalovirus, Epstein-Barr virus, rotavirus, rhinovirus, adenovirus, papillomavirus, poliovirus, mumps, rabies, rubella, coxsackieviruses, equine encephalitis, Japanese encephalitis, yellow fever, Rift Valley fever, hepatitis A, B, C, D, and E virus, and the like; antigens of fungal, protozoan, and parasitic organisms such as Cryptococcus neoformans, Histoplasma capsulatum, Candida albicans, Candida tropicalis, Nocardia asteroides, Rickettsia ricketsii, Rickettsia typhi, Mycoplasma pneumoniae, Chlamydialpsittaci, Chlamydial trachomatis, Plasmodium falciparum, Trypanosoma brucei, Entamoeba histolytica, Toxoplasma gondii, Trichomonas vaginalis, Schistosoma mansoni, and the like. These antigens may be in the form of whole killed organisms, peptides, proteins, glycoproteins, carbohydrates, or combinations thereof.

Those skilled in the art will recognize that this is an exemplary, not comprehensive, list of agents that can be delivered using compositions and methods in accordance with the present invention. Any agent may be associated with nanoparticles for targeted delivery in accordance with the present invention.

### Production of Nanoparticles

Nanoparticle entities in accordance with the invention can be made using any method known in the art. In certain embodiments, the nanoparticle and the modulating entity are physically associated. In certain embodiments, the nanoparticle and the agent to be delivered are physically associated. In certain embodiments, the modulating entity and the agent to be delivered are physically associated. In certain embodiments, the modulating entity, agent to be delivered, and nanoparticle are physically associated.

Physical association can be achieved in a variety of different ways. The physical association may be covalent or non-covalent. The nanoparticle, agent to be delivered, and/or modulating entity may be directly linked to one another, e.g. by one or more covalent bonds, or may be linked by means of one or more linking entities. In some embodiments, the linking entity forms one or more covalent or non-covalent bonds with the nanoparticle and one or more covalent or non-covalent bonds with the agent to be delivered, thereby attaching them to one another. In some embodiments, a first linking entity forms a covalent or non-covalent bond with the nanoparticle and a second linking entity forms a covalent or non-covalent bond with the agent to be delivered. The two linking entities form one or more covalent or non-covalent bond(s) with each other. In some embodiments, the linkage to the nanoparticle will be to the material that forms a coating layer.

In some embodiments, one or more modulating entities, agents to be delivered, and/or other moieties are linked to one another and/or to one or more nanoparticles. The additional moiety can be a biomolecule such as a polypeptide, nucleic acid, polysaccharide, etc.

A variety of methods can be used to attach a biomolecule such as a carbohydrate or polypeptide to a nanoparticle. General strategies include passive adsorption (e.g., via electrostatic interactions), multivalent chelation, high affinity non-covalent binding between members of a specific binding pair, covalent bond formation, etc. (Gao et al., 2005, Curr. Opin. Biotechnol., 16:63; incorporated herein by reference).

A bifunctional cross-linking reagent can be employed. Such reagents contain two reactive groups, thereby providing a means of covalently linking two target groups. The reactive groups in a chemical cross-linking reagent typically belong to various classes of functional groups such as succinimidyl esters, maleimides, and pyridyldisulfides. Exemplary cross-linking agents include, e.g. carbodiimides, N-hydroxysuccinimidyl-4-azidosalicylic acid (NHS-ASA), dimethyl pimelimidate dihydrochloride (DMP), dimethylsuberimidate (DMS), 3,3′-dithiobispropionimidate (DTBP), etc. For example, carbodiimide-mediated amide formation and active ester maleimide-mediated amine and sulfhydryl coupling are widely used approaches.

Common schemes for forming a conjugate involve the coupling of an amine group on one molecule to a thiol group on a second molecule, sometimes by a two- or three-step reaction sequence. A thiol-containing molecule may be reacted with an amine-containing molecule using a heterobifunctional cross-linking reagent, e.g. a reagent containing both a succinimidyl ester and either a maleimide, a pyridyldisulfide, or an iodoacetamide. Amine-carboxylic acid and thiol-carboxylic acid cross-linking, maleimide-sulfhydryl coupling chemistries (e.g., the maleimidobenzoyl-N-hydroxysuccinimide ester (MBS) method), etc., may be used. Polypeptides can conveniently be attached to nanoparticles via amine or thiol groups in lysine or cysteine side chains respectively, or by an N-terminal amino group. Nucleic acids such as RNAs can be synthesized with a terminal amino group. As described in Example 6, the inventors have employed a variety of coupling reagents (e.g., succinimidyl 3-(2-pyridyldithio)propionate (SPDP) and sulfosuccinimidyl-4-(N-maleimidomethyl)cyclohexane-1-carboxylate (sulfo-SMCC) to link QDs and siRNA or to link QDs and peptides. QDs can be prepared with functional groups, e.g., amine or carboxyl groups, available at the surface to facilitate conjugation to a biomolecule. Alternately, moieties such as biotin or streptavidin can be attached to the nanoparticle surface to facilitate binding to moieties functionalized with streptavidin or biotin, respectively.

Non-covalent specific binding interactions can be employed. For example, either the nanoparticle or the biomolecule can be functionalized with biotin with the other being functionalized with streptavidin. These two moieties specifically bind to each other non-covalently and with a high affinity, thereby linking the nanoparticle and the biomolecule. Other specific binding pairs could be similarly used. Alternately, histidine-tagged biomolecules can be conjugated to nanoparticles linked with nickel-nitrolotriaceteic acid (Ni-NTA).

Any biomolecule to be attached to a nanoparticle or RNA may include a spacer. The spacer can be, for example, a short peptide chain, e.g. between 1 and 10 amino acids in length, e.g. 1, 2, 3, 4, or 5 amino acids in length, a nucleic acid, an alkyl chain, etc.

In certain embodiments, a biomolecule is attached to a nanoparticle or agent via a cleavable linkage so that the biomolecule can be removed from the nanoparticle or agent following intracellular delivery. In certain embodiments, a nanoparticle and an RNA (e.g., a short RNAi agent or tRNA) to be delivered in accordance with the invention may be conjugated to one another via a cleavable linkage so that the RNA can be released from the nanoparticle following cellular uptake. Removal or release can occur, for example, as a result of light-directed cleavage, chemical cleavage, protease-mediated cleavage, or enzyme-mediated cleavage. Cleavable linkages include disulfide bonds, acid-labile thioesters, etc. (Oishi et al., 2005, J. Am. Chem. Soc., 127:1624; incorporated herein by reference). Any linker that contains or forms such a bond could be employed. In some embodiments, the linker contains a polypeptide sequence that includes a cleavage site for an intracellular protease.

For additional general information on conjugation methods and cross-linkers, see the journal Bioconjugate Chemistry, published by the American Chemical Society, Columbus Ohio, PO Box 3337, Columbus, Ohio, 43210; “Cross-Linking,” Pierce Chemical Technical Library, available at the Pierce web site and originally published in the 1994-95 Pierce Catalog, and references cited therein; Wong SS, Chemistry of Protein Conjugation and Cross-linking, CRC Press Publishers, Boca Raton, 1991; and Hermanson, G. T., Bioconjugate Techniques, Academic Press, Inc., San Diego, 1996.

It is to be understood that the compositions in accordance with the invention can be made in any suitable manner, and the invention is in no way limited to compositions that can be produced using the methods described herein. Selection of an appropriate method may require attention to the properties of the particular moieties being linked.

If desired, various methods may be used to separate nanoparticles with an attached agent, modulating entity, or other moiety from nanoparticles to which the moiety has not become attached, or to separate nanoparticles having different numbers of moieties attached thereto. For example, size exclusion chromatography or agarose gel electrophoresis can be used to separate populations of nanoparticles having different numbers of moieties attached thereto and/or to separate nanoparticles from other entities. Some methods include size-exclusion or anion-exchange chromatography.

As described further below, in some embodiments, one or more nanoparticles and one or more RNA molecules forms a non-covalent complex with a transfection reagent.

### Cells

In some embodiments, methods in accordance with the present invention may be used to deliver agents to any eukaryotic cell of interest. In certain embodiments, a cell is a mammalian cell. Cells may be of human or non-human origin. For example, they may be of mouse, rat, or non-human primate origin. A cell can be of any cell type. Exemplary cell types include, but are not limited to, endothelial cells, epithelial cells, neurons, hepatocytes, myocytes, chondrocytes, osteoblasts, osteoclasts, lymphocytes, macrophages, neutrophils, fibroblasts, keratinocytes, etc. Cells can be primary cells, immortalized cells, transformed cells, terminally differentiated cells, stem cells (e.g. adult or embryonic stem cells, hematopoietic stem cells), somatic cells, germ cells, etc. Cells can be wild type or mutant cells, e.g., they may have a mutation in one or more genes. Cells may be quiescent or actively proliferating. Cells may be in any stage of the cell cycle. In some embodiments, cells may in the context of a tissue. In some embodiments, cells may be in the context of an organism.

Cells can be normal cells or diseased cells. In certain embodiments, cells are cancer cells, e.g. they originate from a tumor or have been transformed in cell culture (e.g. by transfection with an oncogene). In certain embodiments, cells are infected with a virus or other infectious agent. A virus may be, e.g. a DNA virus, RNA virus, retrovirus, etc. For example, cells can be infected with a human pathogen such as a hepatitis virus, a respiratory virus, human immunodeficiency virus, etc.

Cells may have been experimentally manipulated to overexpress one or more genes of interest, e.g., by transfecting them with an expression vector that contains a coding sequence operably linked to expression signal(s) such as a promoter.

Cells can be cells of a cell line. Exemplary cell lines include HeLa, CHO, COS, BHK, NIH-3T3, HUVEC, etc. For an extensive list of mammalian cell lines, those of ordinary skill in the art may refer to the American Type Culture Collection catalog (ATCC®, Manassas, Va.).

In addition to detection of nanoparticle(s) within cells, the invention provides methods in which cells are optionally analyzed, sorted, and/or manipulated in any of a variety of ways. For example, after a collection of cells has been contacted with a nanoparticle and an RNA, the collection of cells can be separated into two or more populations (sorted), e.g., based on an optical or magnetic signal acquired from individual cells, which reflects the number of nanoparticles contained in the cells.

A variety of different methods for analyzing and separating cells can be used in accordance with the present invention. Such methods are further described in PCT Publication WO 07/67733 (incorporated herein by reference).

### Delivery of Nanoparticles to Cells

Any of a variety of methods may be employed to deliver nanoparticle(s) and RNA to cells.

Electroporation

In certain embodiments, an electric field is applied to enhance intracellular delivery of a nanoparticle sensor component. Application of an electric field to cells to enhance their uptake of DNA, a technique referred to as electroporation, has long been known in the art (Somiari et al., 2002, Mol. Ther., 2:178; and Nikoloff, A., (ed.) Animal Cell Electroporation and Electrofusion Protocols, Methods in Molecular Biology, vol. 48, Humana Press, Totowa, N.J., 1995; both of which are incorporated herein by reference). While not wishing to be bound by any theory, the mechanism may involve temporary disruption of the cell membrane, allowing foreign bodies to enter, followed by resealing of the membrane. In some embodiments, electroporation is used to enhance the uptake of agents (e.g. RNAs) and nanoparticles by cells. Standard electroporation protocols known in the art can be used. Parameters such as electric field strength, voltage, capacitance, duration and number of electric pulse(s), cell number of concentration, and the composition of the solution in which the cells are maintained during or after electroporation can be optimized for the delivery of agents (e.g. RNAs) and of nanoparticles of any particular size, shape, and composition and/or to achieve desired levels of cell viability. In some embodiments, methods in accordance with the invention are not limited to parameters that have been successfully used to enhance cell transfection in the art. Exemplary parameter ranges include, e.g., charging voltages of 100 volts-500 volts and pulse lengths of 0.5 ms-20 ms.

Microinjection

In certain embodiments, cells are microinjected with a composition comprising one or more modulating entities, agents to be delivered, and optically or magnetically detectable nanoparticles. Optionally the agent and the nanoparticle are physically associated. An automated microinjection apparatus can be used (see, e.g., U.S. Pat. No. 5,976,826; incorporated herein by reference).

Pharmaceutical Compositions

The present invention provides nanoparticle entities comprising one or more modulating entities and/or one or more agents to be delivered. In some embodiments, the present invention provides pharmaceutical compositions comprising nanoparticle entities as described herein and one or more pharmaceutically acceptable excipients. Such pharmaceutical compositions may optionally comprise one or more additional therapeutically-active substances. In accordance with some embodiments, a method of administering pharmaceutical compositions comprising nanoparticle entities to a subject in need thereof is provided. In some embodiments, compositions are administered to humans. For the purposes of the present disclosure, the phrase “active ingredient” generally refers to nanoparticle entities as described herein.

Although the descriptions of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals, including commercially relevant mammals such as cattle, pigs, horses, sheep, cats, and/or dogs; and/or birds, including commercially relevant birds such as chickens, ducks, geese, and/or turkeys.

Formulations of the pharmaceutical compositions described herein may be prepared by any method known or hereafter developed in the art of pharmacology. In general, such preparatory methods include the step of bringing the active ingredient into association with an excipient and/or one or more other accessory ingredients, and then, if necessary and/or desirable, shaping and/or packaging the product into a desired single- or multi-dose unit.

A pharmaceutical composition in accordance with the invention may be prepared, packaged, and/or sold in bulk, as a single unit dose, and/or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition in accordance with the invention will vary, depending upon the identity, size, and/or condition of the subject treated and further depending upon the route by which the composition is to be administered. By way of example, the composition may comprise between 0.1% and 100% (w/w) active ingredient.

Pharmaceutical formulations may additionally comprise a pharmaceutically acceptable excipient, which, as used herein, includes any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired. Remington's The Science and Practice of Pharmacy, 21^{st} Edition, A. R. Gennaro, (Lippincott, Williams & Wilkins, Baltimore, Md., 2006) discloses various excipients used in formulating pharmaceutical compositions and known techniques for the preparation thereof. Except insofar as any conventional excipient medium is incompatible with a substance or its derivatives, such as by producing any undesirable biological effect or otherwise interacting in a deleterious manner with any other component(s) of the pharmaceutical composition, its use is contemplated to be within the scope of this invention.

In some embodiments, a pharmaceutically acceptable excipient is at least 95%, at least 96%, at least 97%, at least 98%, at least 99%, or 100% pure. In some embodiments, an excipient is approved for use in humans and for veterinary use. In some embodiments, an excipient is approved by United States Food and Drug Administration. In some embodiments, an excipient is pharmaceutical grade. In some embodiments, an excipient meets the standards of the United States Pharmacopoeia (USP), the European Pharmacopoeia (EP), the British Pharmacopoeia, and/or the International Pharmacopoeia.

Pharmaceutically acceptable excipients used in the manufacture of pharmaceutical compositions include, but are not limited to, inert diluents, dispersing and/or granulating agents, surface active agents and/or emulsifiers, disintegrating agents, binding agents, preservatives, buffering agents, lubricating agents, and/or oils. Such excipients may optionally be included in pharmaceutical formulations. Excipients such as cocoa butter and suppository waxes, coloring agents, coating agents, sweetening, flavoring, and/or perfuming agents can be present in the composition, according to the judgment of the formulator.

Exemplary diluents include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and/or combinations thereof.

Exemplary granulating and/or dispersing agents include, but are not limited to, potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, etc., and/or combinations thereof.

Exemplary surface active agents and/or emulsifiers include, but are not limited to, natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum® [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxyvinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween®20], polyoxyethylene sorbitan [Tween®60], polyoxyethylene sorbitan monooleate [Tween®80], sorbitan monopalmitate [Span®40], sorbitan monostearate [Span®60], sorbitan tristearate [Span®65], glyceryl monooleate, sorbitan monooleate [Span® 80]), polyoxyethylene esters (e.g. polyoxyethylene monostearate [Myrj®45], polyoxyethylene hydrogenated castor oil, polyethoxylated castor oil, polyoxymethylene stearate, and Solutol®), sucrose fatty acid esters, polyethylene glycol fatty acid esters (e.g. Cremophor®), polyoxyethylene ethers, (e.g. polyoxyethylene lauryl ether [Brij®30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic®F 68, Poloxamer®188, cetrimonium bromide, cetylpyridinium chloride, benzalkonium chloride, docusate sodium, etc. and/or combinations thereof.

Exemplary binding agents include, but are not limited to, starch (e.g. cornstarch and starch paste); gelatin; sugars (e.g. sucrose, glucose, dextrose, dextrin, molasses, lactose, lactitol, mannitol,); natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum™), and larch arabogalactan); alginates; polyethylene oxide; polyethylene glycol; inorganic calcium salts; silicic acid; polymethacrylates; waxes; water; alcohol; etc.; and combinations thereof.

Exemplary preservatives may include, but are not limited to, antioxidants, chelating agents, antimicrobial preservatives, antifungal preservatives, alcohol preservatives, acidic preservatives, and/or other preservatives. Exemplary antioxidants include, but are not limited to, alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and/or sodium sulfite. Exemplary chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and/or trisodium edetate. Exemplary antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and/or thimerosal. Exemplary antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and/or sorbic acid. Exemplary alcohol preservatives include, but are not limited to, ethanol, polyethylene glycol, phenol, phenolic compounds, bisphenol, chlorobutanol, hydroxybenzoate, and/or phenylethyl alcohol. Exemplary acidic preservatives include, but are not limited to, vitamin A, vitamin C, vitamin E, beta-carotene, citric acid, acetic acid, dehydroacetic acid, ascorbic acid, sorbic acid, and/or phytic acid. Other preservatives include, but are not limited to, tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant Plus®, Phenonip®, methylparaben, Germall®115, Germaben®II, Neolone™, Kathon™, and/or Euxyl®.

Exemplary buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, etc., and/or combinations thereof.

Exemplary lubricating agents include, but are not limited to, magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, etc., and combinations thereof.

Exemplary oils include, but are not limited to, almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and/or combinations thereof.

Liquid dosage forms for oral and parenteral administration include, but are not limited to, pharmaceutically acceptable emulsions, microemulsions, solutions, suspensions, syrups, and/or elixirs. In addition to active ingredients, liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, oral compositions can include adjuvants such as wetting agents, emulsifying and suspending agents, sweetening, flavoring, and/or perfuming agents. In certain embodiments for parenteral administration, compositions are mixed with solubilizing agents such an Cremopho®, alcohols, oils, modified oils, glycols, polysorbates, cyclodextrins, polymers, and/or combinations thereof.

Injectable preparations, for example, sterile injectable aqueous or oleaginous suspensions may be formulated according to the known art using suitable dispersing agents, wetting agents, and/or suspending agents. Sterile injectable preparations may be sterile injectable solutions, suspensions, and/or emulsions in nontoxic parenterally acceptable diluents and/or solvents, for example, as a solution in 1,3-butanediol. Among the acceptable vehicles and solvents that may be employed are water, Ringer's solution, U.S.P., and isotonic sodium chloride solution. Sterile, fixed oils are conventionally employed as a solvent or suspending medium. For this purpose any bland fixed oil can be employed including synthetic mono- or diglycerides. Fatty acids such as oleic acid can be used in the preparation of injectables.

Injectable formulations can be sterilized, for example, by filtration through a bacterial-retaining filter, and/or by incorporating sterilizing agents in the form of sterile solid compositions which can be dissolved or dispersed in sterile water or other sterile injectable medium prior to use.

In order to prolong the effect of an active ingredient, it is often desirable to slow the absorption of the active ingredient from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are prepared by entrapping the drug in liposomes or microemulsions which are compatible with body tissues.

Compositions for rectal or vaginal administration are typically suppositories which can be prepared by mixing compositions with suitable non-irritating excipients such as cocoa butter, polyethylene glycol or a suppository wax which are solid at ambient temperature but liquid at body temperature and therefore melt in the rectum or vaginal cavity and release the active ingredient.

Solid dosage forms for oral administration include capsules, tablets, pills, powders, and granules. In such solid dosage forms, the active ingredient is mixed with at least one inert, pharmaceutically acceptable excipient such as sodium citrate or dicalcium phosphate and/or fillers or extenders (e.g. starches, lactose, sucrose, glucose, mannitol, and silicic acid), binders (e.g. carboxymethylcellulose, alginates, gelatin, polyvinylpyrrolidinone, sucrose, and acacia), humectants (e.g. glycerol), disintegrating agents (e.g. agar, calcium carbonate, potato or tapioca starch, alginic acid, certain silicates, and sodium carbonate), solution retarding agents (e.g. paraffin), absorption accelerators (e.g. quaternary ammonium compounds), wetting agents (e.g. cetyl alcohol and glycerol monostearate), absorbents (e.g. kaolin and bentonite clay), and lubricants (e.g. talc, calcium stearate, magnesium stearate, solid polyethylene glycols, sodium lauryl sulfate), and mixtures thereof. In the case of capsules, tablets and pills, the dosage form may comprise buffering agents.

Solid compositions of a similar type may be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally comprise opacifying agents and can be of a composition that they release the active ingredient(s) only, or preferentially, in a certain part of the intestinal tract, optionally, in a delayed manner. Examples of embedding compositions which can be used include polymeric substances and waxes. Solid compositions of a similar type may be employed as fillers in soft and hard-filled gelatin capsules using such excipients as lactose or milk sugar as well as high molecular weight polyethylene glycols and the like.

Dosage forms for topical and/or transdermal administration of a composition may include ointments, pastes, creams, lotions, gels, powders, solutions, sprays, inhalants and/or patches. Generally, the active ingredient is admixed under sterile conditions with a pharmaceutically acceptable excipient and/or any needed preservatives and/or buffers as may be required. Additionally, the present invention contemplates the use of transdermal patches, which often have the added advantage of providing controlled delivery of a compound to the body. Such dosage forms may be prepared, for example, by dissolving and/or dispensing the compound in the proper medium. Alternatively or additionally, the rate may be controlled by either providing a rate controlling membrane and/or by dispersing the compound in a polymer matrix and/or gel.

Suitable devices for use in delivering intradermal pharmaceutical compositions described herein include short needle devices such as those described in U.S. Pat. Nos. 4,886,499; 5,190,521; 5,328,483; 5,527,288; 4,270,537; 5,015,235; 5,141,496; and 5,417,662. Intradermal compositions may be administered by devices which limit the effective penetration length of a needle into the skin, such as those described in PCT publication WO 99/34850 and functional equivalents thereof. Jet injection devices which deliver liquid vaccines to the dermis via a liquid jet injector and/or via a needle which pierces the stratum corneum and produces a jet which reaches the dermis are suitable. Jet injection devices are described, for example, in U.S. Pat. Nos. 5,480,381; 5,599,302; 5,334,144; 5,993,412; 5,649,912; 5,569,189; 5,704,911; 5,383,851; 5,893,397; 5,466,220; 5,339,163; 5,312,335; 5,503,627; 5,064,413; 5,520,639; 4,596,556; 4,790,824; 4,941,880; 4,940,460; and PCT publications WO 97/37705 and WO 97/13537. Ballistic powder/particle delivery devices which use compressed gas to accelerate vaccine in powder form through the outer layers of the skin to the dermis are suitable. Alternatively or additionally, conventional syringes may be used in the classical mantoux method of intradermal administration.

Formulations suitable for topical administration include, but are not limited to, liquid and/or semi liquid preparations such as liniments, lotions, oil in water and/or water in oil emulsions such as creams, ointments and/or pastes, and/or solutions and/or suspensions. Topically-administrable formulations may, for example, comprise from about 1% to about 10% (w/w) active ingredient, although the concentration of the active ingredient may be as high as the solubility limit of the active ingredient in the solvent. Formulations for topical administration may further comprise one or more of the additional ingredients described herein.

A pharmaceutical composition may be prepared, packaged, and/or sold in a formulation suitable for pulmonary administration via the buccal cavity. Such a formulation may comprise dry particles which comprise the active ingredient and which have a diameter in the range from about 0.5 nm to about 7 nm or from about 1 nm to about 6 nm. Such compositions are conveniently in the form of dry powders for administration using a device comprising a dry powder reservoir to which a stream of propellant may be directed to disperse the powder and/or using a self propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nm and at least 95% of the particles by number have a diameter less than 7 nm. Alternatively, at least 95% of the particles by weight have a diameter greater than 1 nm and at least 90% of the particles by number have a diameter less than 6 nm. Dry powder compositions may include a solid fine powder diluent such as sugar and are conveniently provided in a unit dose form.

Low boiling propellants generally include liquid propellants having a boiling point of below 65° F. at atmospheric pressure. Generally the propellant may constitute 50% to 99.9% (w/w) of the composition, and the active ingredient may constitute 0.1% to 20% (w/w) of the composition. The propellant may further comprise additional ingredients such as a liquid non-ionic and/or solid anionic surfactant and/or a solid diluent (which may have a particle size of the same order as particles comprising the active ingredient).

Pharmaceutical compositions formulated for pulmonary delivery may provide the active ingredient in the form of droplets of a solution and/or suspension. Such formulations may be prepared, packaged, and/or sold as aqueous and/or dilute alcoholic solutions and/or suspensions, optionally sterile, comprising the active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. The droplets provided by this route of administration may have an average diameter in the range from about 0.1 nm to about 200 nm.

The formulations described herein as being useful for pulmonary delivery are useful for intranasal delivery of a pharmaceutical composition. Another formulation suitable for intranasal administration is a coarse powder comprising the active ingredient and having an average particle from about 0.2 μm to 500 μm. Such a formulation is administered in the manner in which snuff is taken, i.e. by rapid inhalation through the nasal passage from a container of the powder held close to the nose.

Formulations suitable for nasal administration may, for example, comprise from about as little as 0.1% (w/w) and as much as 100% (w/w) of the active ingredient, and may comprise one or more of the additional ingredients described herein. A pharmaceutical composition may be prepared, packaged, and/or sold in a formulation suitable for buccal administration. Such formulations may, for example, be in the form of tablets and/or lozenges made using conventional methods, and may, for example, 0.1% to 20% (w/w) active ingredient, the balance comprising an orally dissolvable and/or degradable composition and, optionally, one or more of the additional ingredients described herein. Alternately, formulations suitable for buccal administration may comprise a powder and/or an aerosolized and/or atomized solution and/or suspension comprising the active ingredient. Such powdered, aerosolized, and/or aerosolized formulations, when dispersed, may have an average particle and/or droplet size in the range from about 0.1 nm to about 200 nm, and may further comprise one or more of the additional ingredients described herein.

A pharmaceutical composition may be prepared, packaged, and/or sold in a formulation suitable for ophthalmic administration. Such formulations may, for example, be in the form of eye drops including, for example, a 0.1/1.0% (w/w) solution and/or suspension of the active ingredient in an aqueous or oily liquid excipient. Such drops may further comprise buffering agents, salts, and/or one or more other of the additional ingredients described herein. Other opthalmically-administrable formulations which are useful include those which comprise the active ingredient in microcrystalline form and/or in a liposomal preparation. Ear drops and/or eye drops are contemplated as being within the scope of this invention.

General considerations in the formulation and/or manufacture of pharmaceutical agents may be found, for example, in Remington: The Science and Practice of Pharmacy 21^{st} ed., Lippincott Williams & Wilkins, 2005.

Administration to a Subject

Compositions, according to the method of the present invention, may be administered to a subject using any amount and any route of administration effective for treating a disease, disorder, and/or condition. The exact amount required will vary from subject to subject, depending on the species, age, and general condition of the subject, the severity of the infection, the particular composition, its mode of administration, its mode of activity, and the like. Compositions in accordance with the invention are typically formulated in dosage unit form for ease of administration and uniformity of dosage. It will be understood, however, that the total daily usage of the compositions of the present invention will be decided by the attending physician within the scope of sound medical judgment. The specific therapeutically effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

Pharmaceutical compositions may be administered to animals, such as mammals (e.g., humans, domesticated animals, cats, dogs, mice, rats, etc.). In some embodiments, pharmaceutical compositions are administered to humans. The pharmaceutical compositions in accordance with the present invention may be administered by any route. In some embodiments, pharmaceutical compositions of the present invention are administered by a variety of routes, including oral, intravenous, intramuscular, intra-arterial, intramedullary, intrathecal, subcutaneous, intraventricular, transdermal, interdermal, rectal, intravaginal, intraperitoneal, topical (e.g. by powders, ointments, creams, gels, lotions, and/or drops), mucosal, nasal, buccal, enteral, vitreal, intratumoral, sublingual; by intratracheal instillation, bronchial instillation, and/or inhalation; as an oral spray, nasal spray, and/or aerosol, and/or through a portal vein catheter. In some embodiments, pharmaceutical compositions are administered by systemic intravenous injection, regional administration via blood and/or lymph supply, and/or direct administration to an affected site (e.g. a therapeutic implant, such as a hydrogel). In specific embodiments, thermally-responsive conjugates in accordance with the present invention and/or pharmaceutical compositions thereof may be administered intravenously. In specific embodiments, nanoparticle entities in accordance with the present invention and/or pharmaceutical compositions thereof may be administered intraperitoneally. In specific embodiments, nanoparticle entities in accordance with the present invention and/or pharmaceutical compositions thereof may be administered intrathecally. In specific embodiments, nanoparticle entities in accordance with the present invention and/or pharmaceutical compositions thereof may be administered intratumorally. In specific embodiments, nanoparticle entities in accordance with the present invention and/or pharmaceutical compositions thereof may be administered intramuscularly. In specific embodiments, nanoparticle entities in accordance with the present invention and/or pharmaceutical compositions thereof may be administered via vitreal administration. In specific embodiments, nanoparticle entities in accordance with the present invention and/or pharmaceutical compositions thereof may be administered via a portal vein catheter. In specific embodiments, nanoparticle entities in accordance with the present invention and/or pharmaceutical compositions thereof may be immobilized into a hydrogel for controlled long-term release of nanoparticle entities. However, the invention encompasses the delivery of nanoparticle entities and/or pharmaceutical compositions thereof by any appropriate route taking into consideration likely advances in the sciences of drug delivery.

In general the most appropriate route of administration will depend upon a variety of factors including the nature of the agent (e.g., its stability in the environment of the gastrointestinal tract), the condition of the patient (e.g. whether the patient is able to tolerate oral administration), etc. The invention encompasses the delivery of the pharmaceutical compositions by any appropriate route taking into consideration likely advances in the sciences of drug delivery.

In certain embodiments, compositions in accordance with the invention may be administered parenterally at dosage levels sufficient to deliver from about 0.001 mg/kg to about 100 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, or from about 1 mg/kg to about 25 mg/kg, of subject body weight per day, one or more times a day, to obtain the desired therapeutic effect. The desired dosage may be delivered three times a day, two times a day, once a day, every other day, every third day, every week, every two weeks, every three weeks, or every four weeks. In certain embodiments, the desired dosage may be delivered using multiple administrations (e.g., two, three, four, five, six, seven, eight, nine, ten, eleven, twelve, thirteen, fourteen, or more administrations).

Nanoparticles and pharmaceutical compositions in accordance with the present invention may be administered either alone or in combination with one or more other therapeutic agents. By “in combination with,” it is not intended to imply that the agents must be administered at the same time and/or formulated for delivery together, although these methods of delivery are within the scope of the invention. Compositions can be administered concurrently with, prior to, or subsequent to, one or more other desired therapeutics or medical procedures. In general, each agent will be administered at a dose and/or on a time schedule determined for that agent. In some embodiments the invention encompasses the delivery of pharmaceutical compositions in combination with agents that may improve their bioavailability, reduce and/or modify their metabolism, inhibit their excretion, and/or modify their distribution within the body.

The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, a composition useful for treating cancer in accordance with the invention may be administered concurrently with another anticancer agent), or they may achieve different effects (e.g. control of any adverse effects).

Nanoparticles and/or pharmaceutical compositions in accordance with the present invention may be administered alone and/or in combination with other nanoparticles and/or agents for treatment of a disease, disorder, or condition. In will further be appreciated that therapeutically active agents utilized in combination may be administered together in a single composition or administered separately in different compositions. In general, it is expected that agents utilized in combination with be utilized at levels that do not exceed the levels at which they are utilized individually. In some embodiments, the levels utilized in combination will be lower than those utilized individually.

### Applications

Methods in accordance with the invention may be used to alter or affect the delivery of nanoparticles to specific tissues, cells, and/or subcellular locales. In some embodiments, delivery of nanoparticles is used to deliver one or more therapeutic, diagnostic, and/or prophylactic agents. In certain embodiments, the targeted cells are cancer cells, and the agent to be delivered is one or more anti-cancer agents. In certain embodiments, targeted cells are cells that have been infected with a virus, and the agent to be delivered is one or more anti-viral agents. In some embodiments, the virus may be, for example, a DNA virus, RNA virus, retrovirus, etc. In some embodiments, the cells can be infected with a human pathogen such as a hepatitis virus, a respiratory virus, human immunodeficiency virus, etc. In some embodiments, the targeted cells are liver cells, and the agent to be delivered is one or more agents useful for treating liver diseases (e.g. hepatocellular carcinoma; fibrosis/cirrhosis; genetic defects; metabolic and clotting disorders, such as diabetes and obesity that are mediated through the liver; hepatitis, such as hepatitis A, B, C, and/or D; other infectious diseases, such as malaria, dengue, etc.; etc.).

In certain embodiments, nanoparticles and/or agents to be delivered are targeted to specific subcellular locales. For example, nanoparticles and/or agents may be targeted for sequestration within an endosome. In some embodiments, nanoparticles and/or agents to be delivered are sequestered in endosomal compartments for a period of minutes, hours, days, weeks, or months. In some embodiments, the nanoparticles and/or agents may be released from the endosome in response to a “trigger.” The trigger is used to release the nanoparticle from endosome entrapment at a later time. Until release, the nanoparticle and/or agents remain dormant. Triggers can be in form of heat, light (e.g., UV, visible, near-infrared), electromagnetic radiation, or a chemical. Exemplary chemicals that can trigger endosomal release include, but are not limited to, choloroquine, cationic liposomes, cationic polymers, proton pump inhibitors. These triggers can affect the endosome compartment directly (e.g., by affecting pore formation or endosomal lysis) and/or can provide energy input to the nanoparticle and/or agents, which is used to disrupt the endosomal membrane.

In some embodiments, the agent to be delivered is an RNAi entity. In some embodiments, the RNAi entity is sequestered in an endosome until a trigger is presented, thereby controlling the release of the RNAi entity from the endosome. In some embodiments, such a method is used to spatially and temporally control the activity of an RNAi entity.

In certain embodiments, nanoparticles are associated with one or more entities that mediate controlled release of an agent. In some embodiments, an agent and targeting peptide are conjugated to nanoparticles via protease-cleavable peptides. Cleavage will occur the sites where corresponding proteases are present. Proteases such as matrix metalloproteases (MMPs) are upregulated in many types of tumors. Therefore, agents to be delivered that are conjugated to nanoparticle entities via protease-cleavable bonds are released from nanoparticles when nanoparticles reach tumor sites in vivo.

In some embodiments, the cleavable peptide sequence, protease, and disease to be treated are selected from Table 1. In some embodiments, proteases that could serve as target proteases according to the present invention include, but are not limited to, any matrix metalloprotease (e.g. MMP-1, MMP-7, MMP-9, MMP-13, etc.), Caspase-2, NFκB, Cathespin S, Cathespin K, etc.

The invention encompasses in vivo applications of the compositions and methods described herein. In certain embodiments, a composition comprising a detectable nanoparticle, e.g., a QD, and an agent (e.g., an RNAi entity) is administered to a subject. Any of the detectable nanoparticles described herein may be used. For example, in some embodiments, the nanoparticle and the agent to be delivered are conjugated to one another. In some embodiments, a modulating entity such as a translocation peptide is conjugated to the nanoparticle. The in vivo applications encompass administering one or more nanoparticles to a subject for targeted delivery of an agent to specific tissues, cells, and/or subcellular locales.

Following administration to the subject the nanoparticle is detected, thereby providing an indication of the distribution and/or uptake of the agent by various cells, tissues, organs, etc., and optionally providing an indication of the activity of the agent in such cells, tissues, organs, etc. Detection can take place at any suitable time following administration. In some embodiments, a tissue sample (e.g., a tissue section) is obtained from the subject and examined microscopically by any of the techniques described herein. Alternately, individual cells can be isolated from the subject and examined, sorted, or further processed. In vivo imaging techniques such as fluorescence imaging can be employed to detect nanoparticles in a living subject (Gao et al., 2004, Nat. Biotechnol., 22:969; incorporated herein by reference). In vivo administration provides the potential for rapidly evaluating the ability of different delivery vehicles to enhance uptake of an agent in a living organism. In addition to detecting nanoparticles, conventional immunostaining or other techniques can be employed, e.g. to confirm activity of an agent, to gather information about the effect of the agent on the subject, etc.

### Kits

The invention provides a variety of kits for conveniently and/or effectively carrying out methods of the present invention. Inventive kits typically comprise one or more nanoparticle entities comprising at least one modulating entity and/or at least one agent to be delivered. In some embodiments, kits comprise a collection of different nanoparticle entities to be used for different purposes (e.g. diagnostics, treatment, and/or prophylaxis). Typically kits will comprise sufficient amounts of nanoparticles to allow a user to perform multiple treatments of a subject(s) and/or to perform multiple experiments. In some embodiments, kits are supplied with or include one or more nanoparticle entities that have been specified by the purchaser.

Inventive kits may include additional components or reagents. For example, kits may comprise one or more control nanoparticles, e.g., positive control (nanoparticles known to target particular target cells) and negative control (nanoparticles known not to target particular target cells) nanoparticle entities. Other components of inventive kits may include cells, cell culture media, tissue, and/or tissue culture media.

Inventive kits may comprise instructions for use. For example, instructions may inform the user of the proper procedure by which to prepare a pharmaceutical composition comprising nanoparticles and/or the proper procedure for administering the pharmaceutical composition to a subject.

In some embodiments, kits include a number of unit dosages of a pharmaceutical composition comprising thermally-responsive conjugates. A memory aid may be provided, for example in the form of numbers, letters, and/or other markings and/or with a calendar insert, designating the days/times in the treatment schedule in which dosages can be administered. Placebo dosages, and/or calcium dietary supplements, either in a form similar to or distinct from the dosages of the pharmaceutical compositions, may be included to provide a kit in which a dosage is taken every day.

Kits may comprise one or more vessels or containers so that certain of the individual components or reagents may be separately housed. Inventive kits may comprise a means for enclosing the individual containers in relatively close confinement for commercial sale, e.g., a plastic box, in which instructions, packaging materials such as styrofoam, etc., may be enclosed.

In some embodiments, inventive kits comprise one or more nanoparticles comprising at least one modulating entity and/or at least one agent to be delivered in accordance with the present invention. In some embodiments, such a kit is used in the treatment, diagnosis, and/or prophylaxis of a subject suffering from and/or susceptible to a disease, condition, and/or disorder (e.g. cancer). In some embodiments, such a kit comprises (i) a nanoparticle entity that is useful in the treatment of cancer; (ii) a syringe, needle, applicator, etc. for administration of the to a subject; and (iii) instructions for use.

### EXEMPLIFICATION

### Example 1

### Co-Delivery of Quantum Dots and siRNA to Cells Allows Quantitation of siRNA Uptake and Correlation of Gene Silencing with Intracellular Fluorescence

### Materials and Methods

Short Interfering RNA and Quantum Dot Preparation

Pre-designed siRNA was used to selectively silence the Lamin A/C gene (Lmna siRNA #73605, NM_{—}019390, Ambion) and the T-cadherin gene (SMARTpool reagent CDH13, NM_{—}019707, Dharmacon). Fluorescently-labeled Lmna siRNA purchased from Dharmacon was designed with a fluorescein molecule on the 5′ end of the sense strand. The annealed sequences were reconstituted in nuclease-free water and used at a concentration of 100 nM (Lmna siRNA, 5′-Fluorescein-Lmna siRNA) or 50 nM (T-cad siRNA).

Green (560 nm emission maxima) and orange (600 nm emission maxima) CdSe-core, ZnS-shell nanocrystals were synthesized and water-solubilized with mercaptoacetic acid (MAA) as previously described (Chan and Nie, 1998, Science, 281:2016; Hines and Guyot-Sionnest, 1996, J. Phys. Chem., 100:468; and Dabbousi et al., 1997, J. Phys. Chem. B, 101:9463; all of which are incorporated herein by reference). MAA-QDs were then surface-modified by reacting with polyethylene glycol (PEG)-thiol MW 5000 (Nektar) overnight at room temperature. Excess PEG-thiol was removed by spin filtration (100 kDa cutoff). QDs are also available commercially as an alternative to synthesis (Quantum Dot Corporation, Evident Technologies). Unless stated otherwise, 5 μg PEGylated QD was used per cell transfection.

Fibroblast Cell Culture and Transfection

3T3-J2 fibroblasts were provided by Howard Green (Harvard Medical School, Cambridge, Mass.; Rheinwald and Green, 1975, Cell, 6:331; incorporated herein by reference) and cultured at 37° C., 5% CO₂ in Dulbecco's Modified Eagle Medium (DMEM) with high glucose, 10% fetal bovine serum (FBS), and 1% penicillin-streptomycin. The transfection procedure was performed using Lipofectamine 2000 (Invitrogen) according to the manufacturer's instructions. Briefly, 3T3 fibroblasts were plated 24 hours prior to transfection at a density of 3×10⁶ cells per 35-mm well, in antibiotic- and serum-free medium. Lipofectamine reagent (5 μl) and either siRNA or QDs were diluted in Dulbecco's Modified Eagles' Medium (DMEM) and complexed at room temperature. For QD/siRNA co-complexes, siRNA and liposomes were allowed to complex for 15 minutes prior to an additional 15 minute incubation with QDs. Complexes were added to cell cultures in fresh antibiotic- and serum-free medium until 5 hours later, at which time the cultures were washed and replaced with regular growth medium. Approximately 24 hours post-transfection, cells were trypsinized and prepared for flow cytometry.

Fluorescence Activated Cell Sorting (FACS)

Flow cytometry and sorting was performed on a FACS Vantage SE flow cytometer (Becton Dickinson) using a 488 nm Ar laser and FL1 bandpass emission (530±20 nm) for the green QDs, FL3 bandpass emission (610±10 nm) for the orange QDs. Fluorescence histograms and dot plots were generated using Cell Quest software (for figures, histograms were re-created using WinMDI software, Scripps Institute, CA). Cell Quest was also used to gate populations of highest and lowest fluorescence intensity for sorting into chilled FBS. Sorted populations were immediately re-plated into separate wells containing regular growth medium and allowed to adhere. Cells were incubated at 37° C. until visualized by fluorescence microscopy or until assayed for protein level.

Western Blotting

Cell cultures were scraped and lysed in RIPA Lysis Buffer (Upstate Biotechnologies) supplemented with COMPLETE EDTA-free Protease inhibitor solution (Roche). Equal amounts (15 μg-20 μg) total protein were loaded onto a 10% Tris-HCl resolving gel, separated by electrophoresis, and transferred to PVDF membrane. The blot was incubated in blocking solution (5% [w/v] nonfat dry milk, 200 mM Tris base [pH 7.4], 5 M NaCl, 5% Tween-20) for 1 hour at room temperature, primary antibody overnight at 4° C., and secondary antibody for 1 hour. Three washes in 200 mM Tris base pH 7.4, 5 M NaCl, 5% Tween-20 took place between steps and after completion of probing. Finally, the blot was visualized by chemiluminscence (Super Signal West Pico Kit, Pierce) and developed. Bands were analyzed for density using MetaMorph Image Analysis software (Universal Imaging) and normalized to loading control (β-actin) bands.

Primary antibodies used were polyclonal lamin A/C antibody (Cell Signaling) at 1:1000 dilution in blocking solution and polyclonal β-actin antibody (Cell Signaling) at 1:750 dilution. T-cadherin primary antibody was a gift from Barbara Ranscht (University of California, San Diego; Ranscht and Dours-Zimmermann, 1991, Neuron, 7:391; incorporated herein by reference). Secondary antibody was goat anti-rabbit IgG-HRP (Santa Cruz Biotechnology) at 1:7500 dilution. Blots were probed simultaneously for lamin A/C protein (70 kDa, 28 kDa) and β-actin protein (45 kDa); after detection, select blots were re-probed for T-cadherin (95 kDa).

Immunofluorescence Staining

Sorted and unsorted cells intended for lamin nuclear protein immunostaining were seeded onto Collagen-I coated glass coverslips. Coverslips with attached cells were washed twice in cold phosphate-buffered saline (PBS, Gibco) and fixed in 4% paraformaldehyde at room temperature. After three brief PBS washes, cells were permeabilized with 0.2% Triton-X for 10 minutes at room temperature and washed again. The cells were blocked with 10% goat serum for 30 minutes at 37° C., incubated in primary antibody (1:100 Lamin A antibody, Santa Cruz Biotechnology) for 90 minutes at 37° C., washed three times with 0.05% Triton-X, incubated in secondary antibody (1:250 AlexaFluor 594 chicken anti-rabbit IgG antibody, Molecular Probes) for 1 hour at room temperature, and washed a final three times. Antibody dilutions were performed in 1% bovine serum albumin (BSA) in PBS. Coverslips were mounted onto glass slides using Vectashield anti-fade medium (Vector Laboratories). Finally, nuclear staining was visualized and documented by phase contrast microscopy or epifluorescence (Nikon Ellipse TE200 inverted fluorescence microscope and CoolSnap-HQ Digital CCD Camera).

### Results

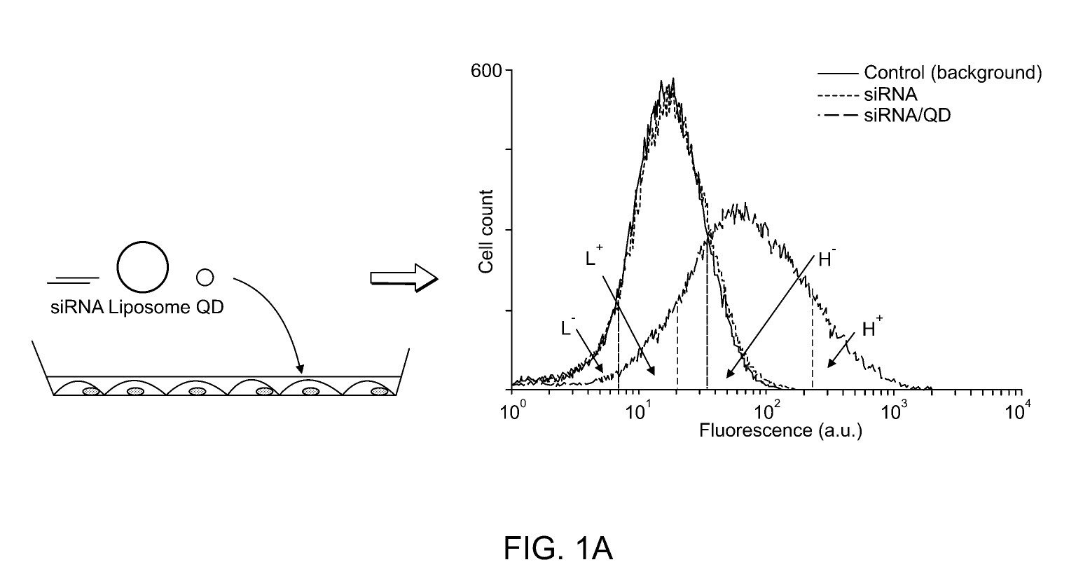

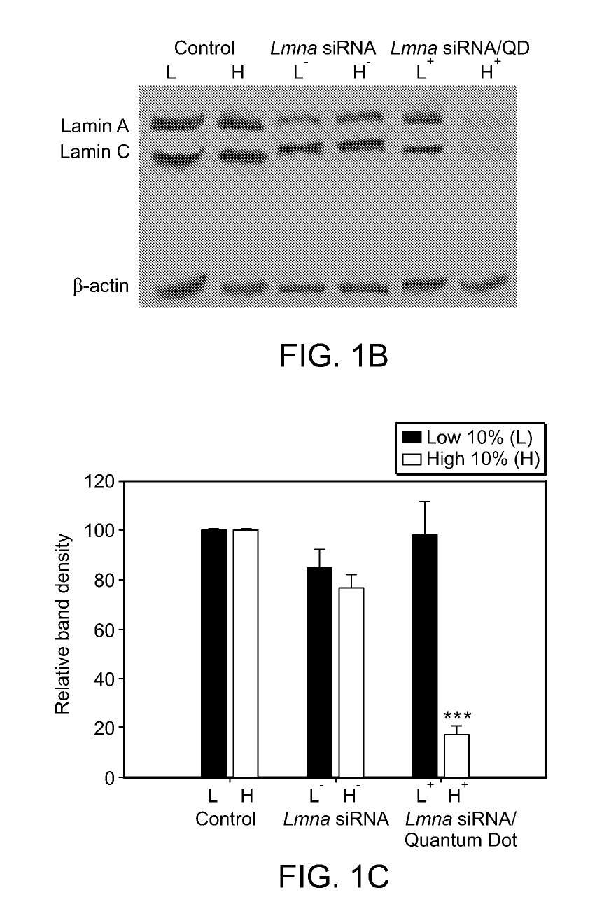

We used cationic liposomes to co-deliver green QDs and siRNA targeting the lamin A/C gene (Lmna) into murine fibroblasts, followed by flow cytometry to quantify intracellular QD uptake (FIG. 1A). The median fluorescence of QD/siRNA-transfected cells compared to mock-transfected cells (liposome reagent only) and cells transfected with siRNA alone varied by approximately 84% (coefficient of variation). FACS was used to gate and collect the brightest 10% (high, H) of each fluorescence distribution, along with the dimmest 10% (low, L).

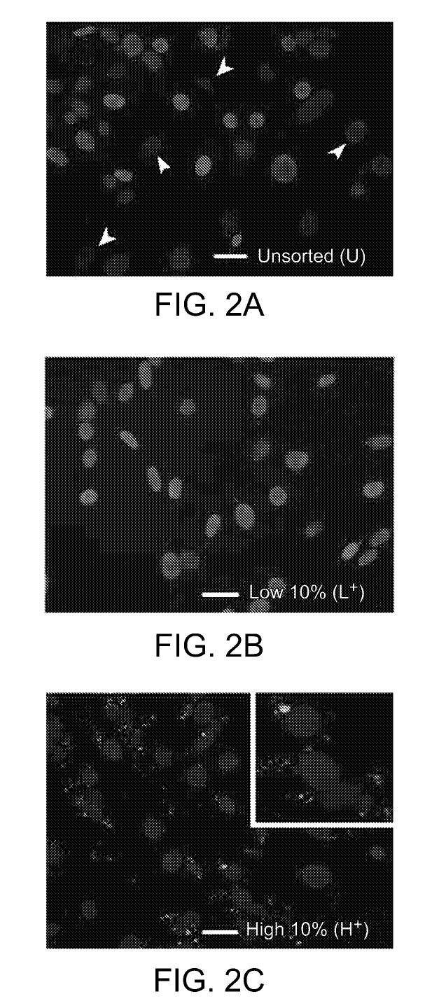

After the sorted cells were re-plated and grown for 72 hours to ensure protein turnover, protein expression analysis by either Western blot or immunostaining was performed. In cells that had been co-transfected with siRNA and QDs, gene silencing correlated directly with intracellular fluorescence. Western blotting (FIG. 1B) and image analysis of lamin A/C protein bands (FIG. 1C) show approximately 90% knockdown in the highly fluorescent cells and negligible knockdown in the dimmest cells. The cells treated with siRNA alone exhibited mediocre gene down-regulation (20%-30%) independent of sorting parameters. Consistent with the quantitative bulk protein assay, immunofluorescent detection of lamin nuclear protein in unsorted, siRNA-transfected cells produced heterogeneous staining throughout the cell population (FIG. 2A). However, in the co-transfected case, the presence of green QDs correlated with consistently weak lamin immunofluorescent staining in the high co-transfected subpopulation (FIG. 2B), compared to a lack of observable QDs and strong lamin staining in the low subpopulation (FIG. 2C). Heterogeneous silencing therefore influences the accuracy of the bulk protein expression readout, suggesting the importance of verifying successful siRNA transfection for each gene knockdown study. Using QDs as photostable probes in combination with FACS, a subpopulation of uniformly-treated cells can be isolated, and also tracked with fluorescence microscopy over long periods of time. This approach is useful for observing the protein downregulation and phenotypic responses of cells to gene regulation over time.

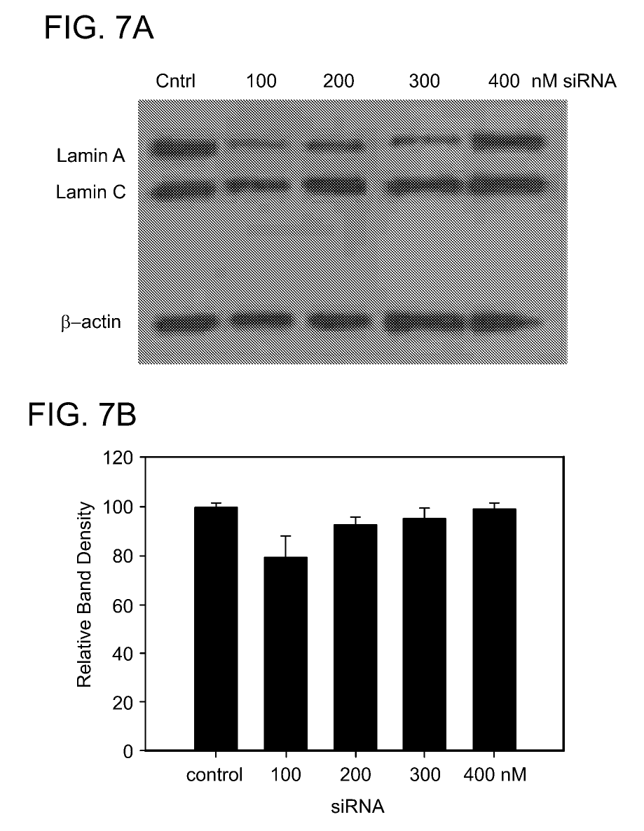

We note that attempts to improve silencing by simply using higher concentrations of siRNA do not improve knockdown but may actually negatively regulate RNAi-mediated gene silencing (FIG. 7; Hong et al., 2005, Biochem J, 390:675; and Kennedy et al., 2004, Nature, 427:645; both of which are incorporated herein by reference). In addition, excesses of either siRNA or cationic liposome has been shown to induce increased cytotoxicity, interferon response (Sledz et al., 2003, Nat. Cell Biol., 5:834; incorporated herein by reference) and “off-target” effects (Jackson et al., 2003, Nat. Biotechnol., 21:635; incorporated herein by reference).

### Example 2

### Optimizing the Correlation Between QD Fluorescence and Gene Silencing

### Materials and Methods

QD and siRNA synthesis, transfection, and Western blotting were performed as described in Example 1.

### Results

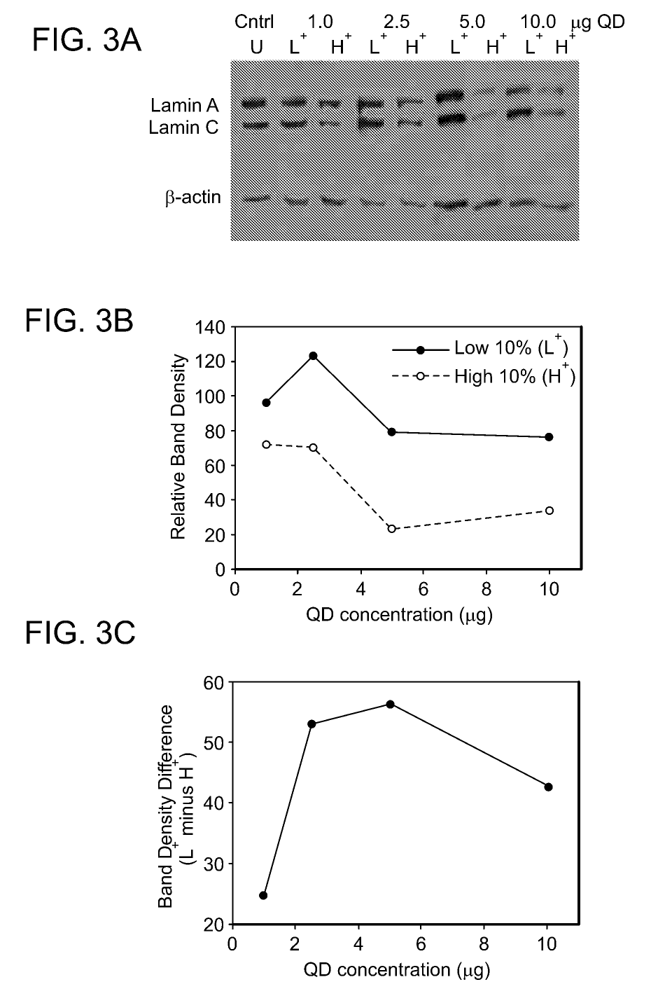

To optimize the QD/siRNA correlative effect, we varied the ratio of QD to lipofection reagent with a fixed dose of 100 nM siRNA. Specifically, we co-complexed Lmna siRNA with QD:lipofection reagent ratios of 1:5, 1:2, 1:1 or 2:1 (corresponding to 1 μg, 2.5 μg, 5 μg, or 10 μg QD) and sorted the high 10% and low 10% of the cell fluorescence distributions as before. We found that optimal fluorescence and gene silencing correlation for the least amount of QD occurs at a 1:1 QD:lipofection reagent mass ratio (5 μg QD), as assayed by Western blot (FIGS. 3A-C). Without wishing to be bound by any theory, we hypothesize that this optimum results from the limited surface area of the cationic liposome delivery agent (approximately 1 μm²) that is shared by the siRNA and QDs during the complexing process. Using too few QDs fails to provide fluorescence that is detectable over background, whereas excess QDs occupy sites on the liposome that would otherwise be available to siRNA. In support of this theory, we found that saturating the liposome with QDs (100:1 ratio) prior to transfection abolished correlation between cellular fluorescence and gene silencing; both high- and low-populations exhibited little to no knockdown (data not shown).

### Example 3

### Multiplexed Assay Allows Simultaneous Monitoring and Sorting of Cells Treated with Different siRNAs

### Materials and Methods

QD and siRNA synthesis, transfection, and Western blotting were performed as described in Example 1.

### Results

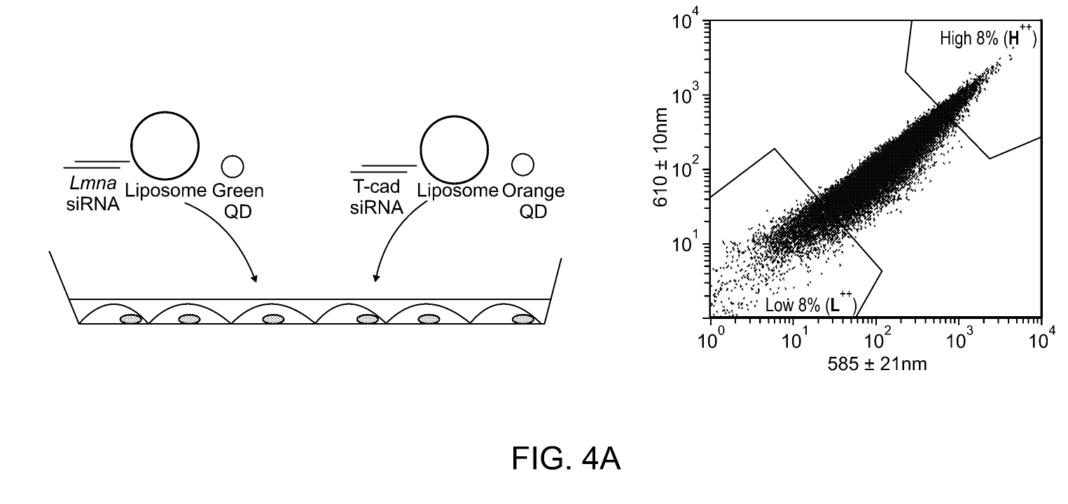

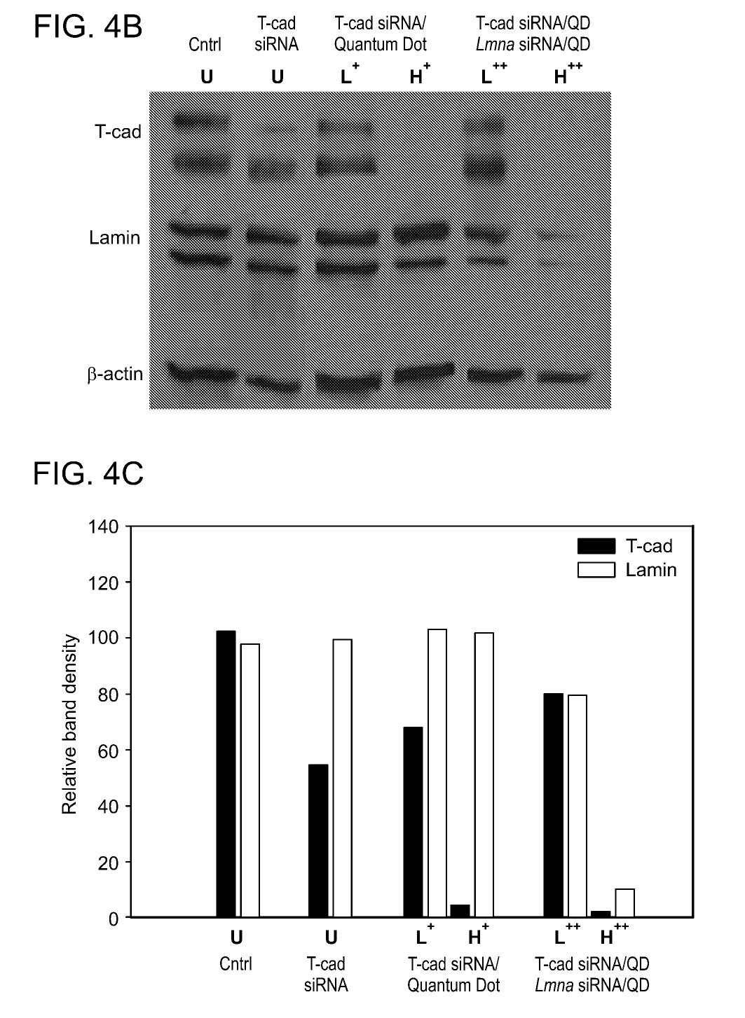

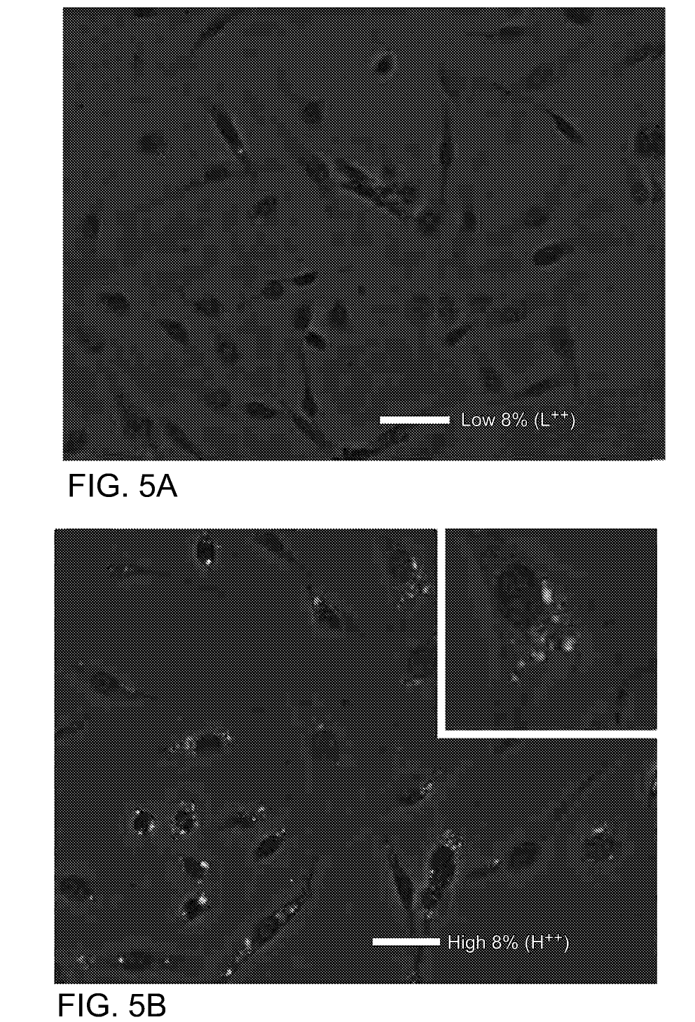

QDs exhibit an extensive range of size- and composition-dependent optical properties, making them highly advantageous for multiplexing (i.e. monitoring and sorting cells that have been treated simultaneously with different siRNA/QD complexes). As a demonstration of these capabilities, we complexed cationic liposomes with either green (em 560 nm) QDs and Lmna siRNA or orange (em 600 nm) QDs and siRNA targeting T-cadherin (T-cad). Cells were exposed simultaneously to both complexes and flow cytometry was used to quantify orange fluorescence (600±10 nm) versus green fluorescence (560±20 nm) (FIG. 4A). Cells exhibiting dual-color fluorescence were gated for low 8% and high 8% fluorescence and collected. Western blots probing lamin A/C and T-cad protein confirm specificity of QD/siRNA complexing (FIGS. 4B,C), while fluorescence microscopy validates gating accuracy and demonstrates multi-color tracking capabilities (FIG. 5). Unsorted cells transfected with T-cad siRNA alone expressed a 45% down-regulation in protein expression quantified by Western blot band densitometry. In contrast, co-delivery of QDs with T-cad siRNA and subsequent sorting enabled separation of the least efficiently transfected cell subpopulation (30% protein knockdown) from a highly transfected population (95% knockdown). In the highest 8% of the dual color, dual siRNA co-transfected cell population, highly effective silencing of both Lmna gene (96% knockdown) and T-cad gene (98% knockdown) was achieved. Given the wide spectrum of QD color possibilities, this method promises to be useful for tracking and sorting multiple siRNA-mediated knockdowns within one cell population.

### Example 4

### Isolation of a Homogeneously Silenced Population of Fibroblasts Reveals a Role for T-Cadherin in Cell-Cell Communication Between Hepatocytes and Non-Parenchymal Cells

### Materials and Methods

QD and siRNA synthesis and transfection were performed as described in Example 1.

Hepatocyte/Fibroblast Co-Cultures

Hepatocytes were isolated from 2 month-3 month old adult female Lewis rats (Charles River Laboratories) and purified as described previously (Seglen, 1976, Methods Cell Biol, 13:29; and Dunn et al., 1991, Biotechnol. Prog., 7:237; both of which are incorporated herein by reference). Fresh, isolated hepatocytes were seeded at a density of 2.5×10⁵ cells per well, in 17-mm wells adsorbed with 0.13 mg/ml Collagen-I. Cultures were maintained at 37° C., 5% CO₂ in hepatocyte medium consisting of DMEM with high glucose, 10% fetal bovine serum, 0.5 U/ml insulin, 7 ng/ml glucagons, 7.5 μg/ml hydrocortisone, 10 U/ml penicillin, and 10 μg/ml streptomycin. 24 hours after hepatocyte seeding, fibroblasts from transfection experiments were co-cultivated at a previously optimized 1:1 hepatocyte:fibroblast ratio in fibroblast medium (Bhatia et al., 1999, FASEB J., 13:1883; incorporated herein by reference). Medium from hepatocyte/fibroblast co-cultures was collected and replaced with hepatocyte medium every 24 hours until completion of the experiment.

Hepatocellular Function Assays

Hepatocyte/fibroblast co-cultures were assayed for albumin production and cytochrome P450 enzymatic activity, prototypic indicators of hepatocellular function (Khetani et al., 2004, Hepatology, 40:545; and Allen et al., 2005, Toxicol Lett, 155:151; both of which are incorporated herein by reference). Albumin content in spent media samples was measured using an enzyme linked immunosorbent assay (ELISA) with horseradish peroxidase detection (Dunn et al., 1991, Biotechnol. Prog., 7:237; incorporated herein by reference). Cytochrome P450 (CYP1A1) enzymatic activity was measured by quantifying the amount of resorufin produced from the CYP-mediated cleavage of ethoxyresorufin O-deethylase (EROD; Behnia et al., 2000, Tissue Eng., 6:467; incorporated herein by reference). Specifically, EROD was incubated with cell cultures for 30 minutes, media was collected, and resorufin fluorescence quantified at 571 nm/585 nm excitation/emission. Error bars represent standard error of the mean (n=3). Statistical significance was determined using one-way ANOVA (analysis of variance).

### Results

The utility of RNAi as a functional genomics tool is predicated upon associating gene silencing with downstream phenotypic observations. Yet non-uniform gene silencing may obscure bulk measurements (protein, mRNA) commonly used to validate gene knockdown and obscure genotype/phenotype correlations. We compared the downstream effects of non-uniform and homogenous gene silencing to specifically examine the stabilizing effect of non-parenchymal cells (3T3 fibroblasts) on hepatocellular function in vitro

Bhatia et al., 1999, FASEB J., 13:1883; incorporated herein by reference). Recently, several cadherins from hepatocyte-fibroblast junctions were identified as potential mediators of liver-specific function in vitro

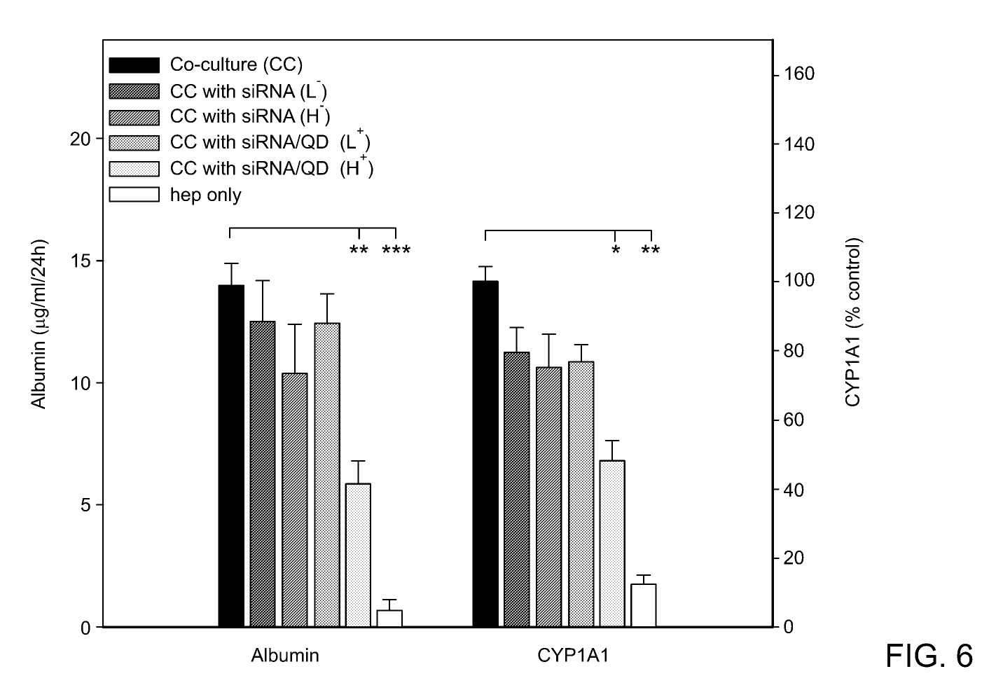

Khetani et al., 2004, Hepatology, 40:545; incorporated herein by reference). Based on this finding, we transfected fibroblasts with T-cad siRNA or T-cad siRNA/QD complexes, sorted each population according to high or low cellular fluorescence, and co-cultivated the populations with hepatocytes. Markers of liver-specific function, albumin synthesis and cytochrome P450 1A1 (CYP1A1) activity, were measured in hepatocyte/3T3 co-cultures (FIG. 6). Compared to control co-cultures, significant downregulation in hepatocellular function (2-fold) was observed exclusively in the cultures that had been treated with T-cad siRNA/QD complexes and sorted for high cellular fluorescence. These studies implicate a role for fibroblast T-cadherin protein expression in modulating hepatocellular function in vitro, an interpretation revealed only once a homogenously-silenced population of fibroblasts was obtained.

### Example 5

### QDs Demonstrate Superior Photostability and Brightness Relative to Fluorescent Dyes for siRNA Tracking

### Materials and Methods

QD and siRNA synthesis and transfection were performed as described in Example 1.

### Results

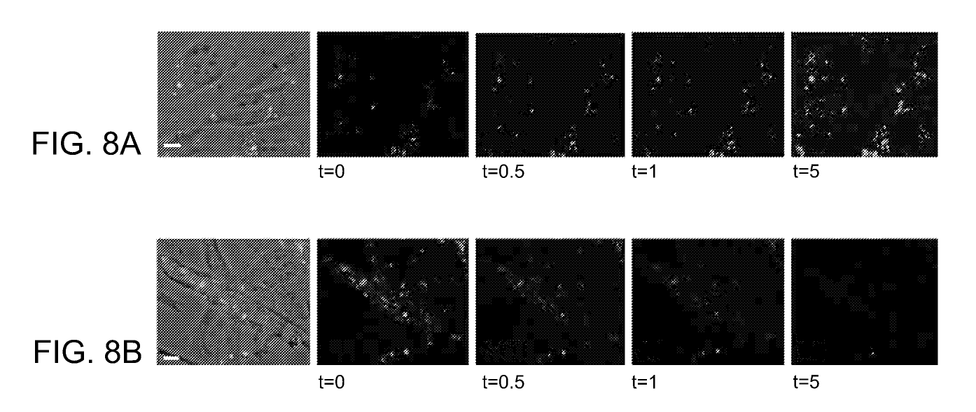

Cells were transfected with 20 μg QD (em 566 nm) or 100 nM Lamin A/C siRNA modified with fluorescein on the 5′ end of the sense strand. As shown in FIG. 8A, QDs fluoresce brightly under continuous mercury lamp exposure over several minutes, while the fluoroscein attached to the siRNA bleaches under continuous excitation and is no longer detectable after t=5 minutes (FIG. 8B).

### Example 6

### Uptake and Silencing Activity of QD/siRNA Conjugates

### Materials and Methods

Quantum dots (Amino PEG ITK 705, Quantum Dot Corporation) were dissolved in 150 mM NaCl, 50 mM Sodium Phosphate, pH 7.2. 300 μg of cross-linker (SPDP, Pierce or SMCC, Sigma) was added per 500 μmol of nanoparticles and allowed to react for 1 hour. After filtering on a NAP5 gravity column to remove excess cross-linker, QDs were added to a 10 fold excess (5 nmol) of thiolated siRNA (first reduced with 0.1 M DTT and then filtered on a NAP5 column). The siRNA used was designed against destabilized enhanced GFP (“EGFP,” Clontech), and thiolated on the 5′ end of the sense strand. After reaction overnight at 4° C., particles were washed twice with PBS, twice with 5×SSC (1.5 M NaCl, 0.15 M Sodium Citrate, pH 7.2), and twice with PBS, using three Amicon-4 (100 kDa cutoff) spin filters. QDs were added to lipofectamine 2000 (1 μl per well of a 24 well plate) and allowed to complex for 20 minutes in serum-free media. QD/lipofectamine complexes were then added to GFP+HeLa cells (20%-40% confluent in a 24 well plate). Media was changed to 10% FBS at 24 hours. Cells were trypsinized and flow cytometry performed at 48 hours to assess GFP and QD signal. Percent knockdown was assessed by comparing with control cells treated with lipofectamine alone.

### Results

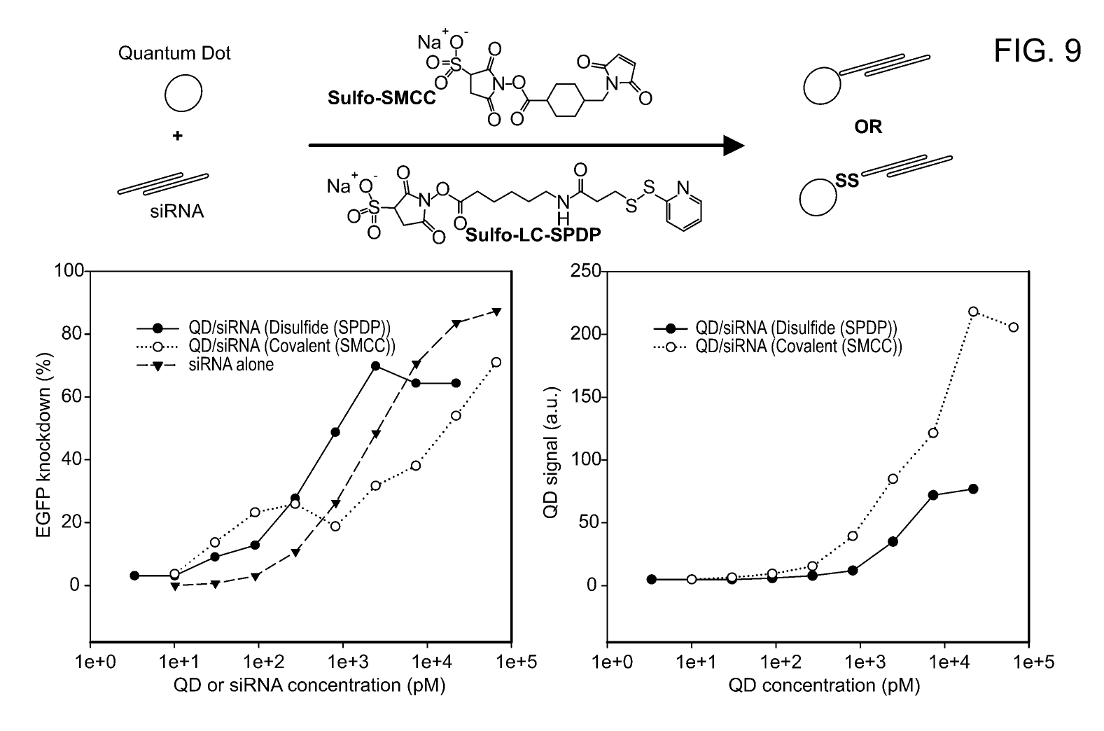

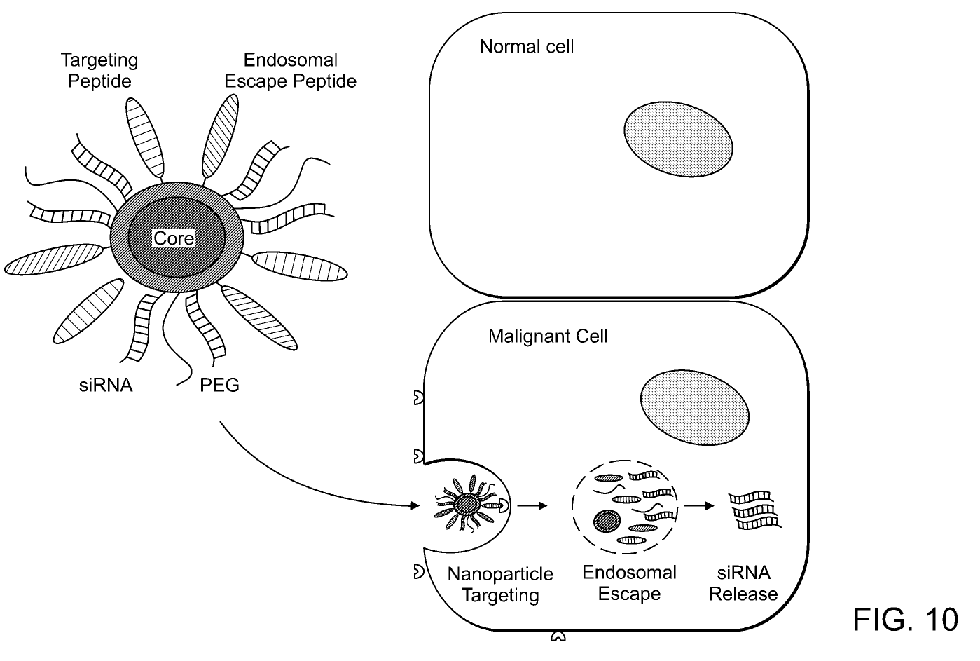

QDs and siRNA targeted to EGFP were conjugated to one another using either sulfo-SMCC or sulfo-LC-SPDP (depicted in the upper portion of FIG. 9) to produce QD/siRNA conjugates. The latter reagent provided conjugation via a disulfide bond. Complexes containing either Lipofectamine and siRNA or Lipofectamine and QD/siRNA conjugates were formed as described above. HeLa cells expressing EGFP were treated with Lipofectamine/siRNA complexes or with either of the two Lipofectamine/QD/siRNA complexes at a range of different QD concentrations. EGFP fluorescence was measured as an indication of EGFP expression. Fluorescence signal from the QD/siRNA complexes was gathered. As shown in FIG. 9 (left panel) both QD/siRNA conjugates resulted in efficient silencing of EGFP, with the disulfide-linked conjugate displaying a greater silencing effect under these conditions although the QD/siRNA conjugates produced using SMCC were taken up in higher amounts by the cells as shown in FIG. 9 (right panel). The apparently greater efficacy of the disulfide-linked conjugates may reflect release of the siRNA from the QD inside the cells.

### Example 7

### Targeted Delivery of QDs to Cells

### Materials and Methods

Quantum dots were conjugated to various peptides using sulfo-SMCC and the procedure described in Example 6 above. Briefly, 300 μg of cross-linker was added to 500 μmol of quantum dots. After 1 hour at room temperature, QDs were filtered on a NAP5 column and added to various thiolated peptides: KAREC (SEQ ID NO: 12), INF7, F3, F3+INF7 (equal molar ratio). KAREC denotes a 5 amino acid peptide, which is used as a non-internalizing control. 100 nM concentration of QDs were added to HeLa cells in media with 10% FBS. “No QDs” indicates no quantum dots were added to the cells and represents the background signal. “No peptide” indicates no peptide was added to the QDs after the cross-linker was added and particles filtered. Four hours later, cells were washed, trypsinized and flow cytometry was performed.

### Results

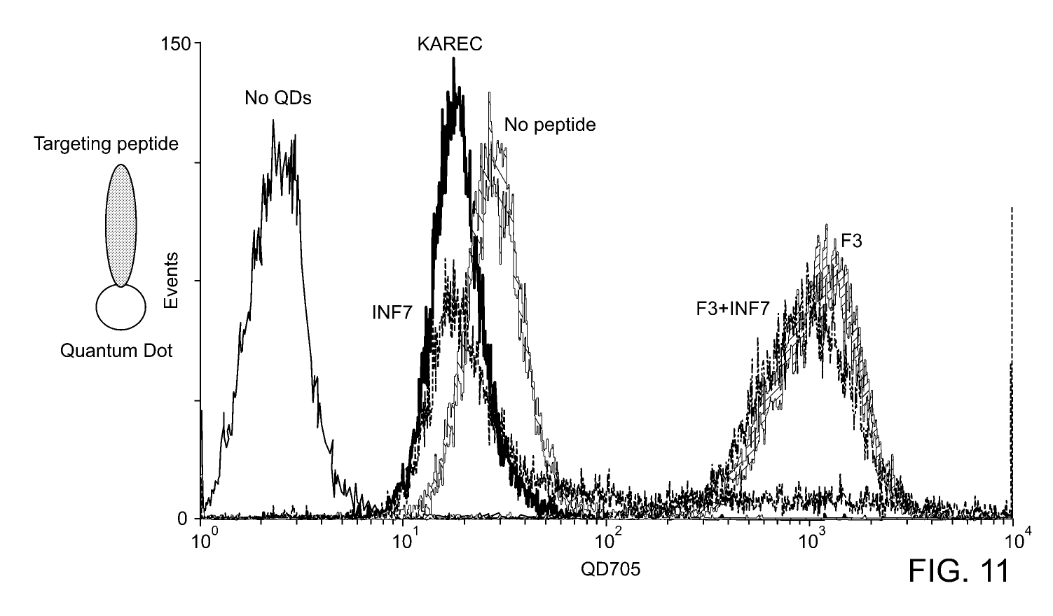

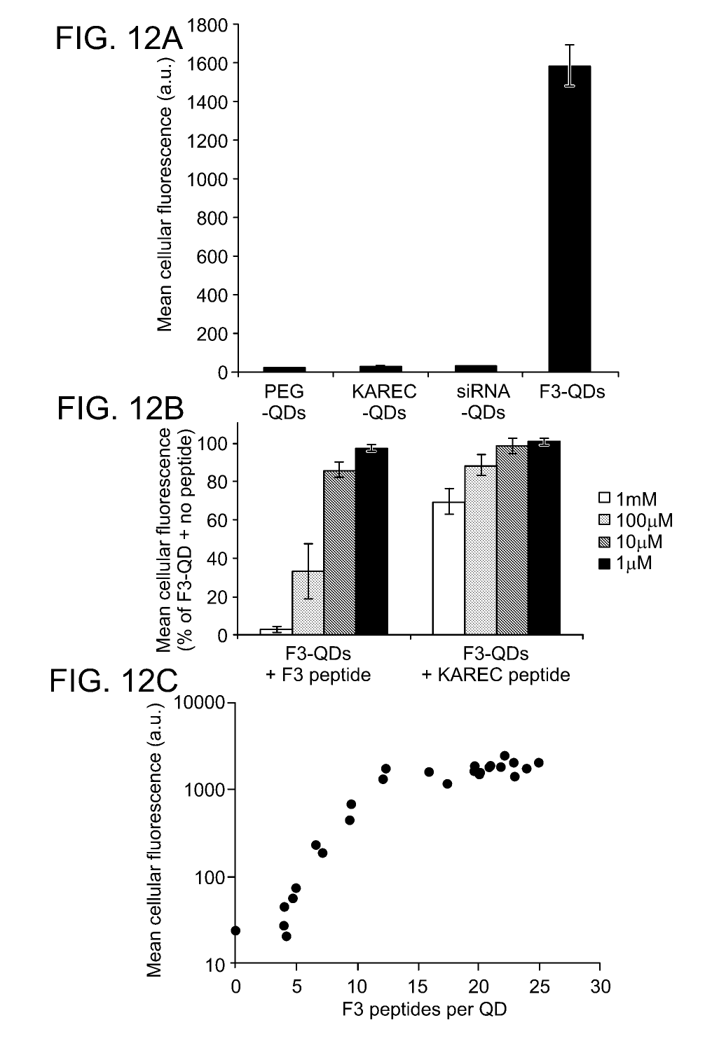

F3 (CAKVKDEPQRRSARLSAKPAPPKPEPKPKKAPAKK, SEQ ID: 13) is a 34 amino acid basic peptide that binds to nucleolin, a protein that is present at higher levels on the surface of dividing than non-dividing cells. INF7 (GLFEAIEGFI ENGWEGMI DGWYGC, SEQ ID NO: 14) is a peptide derived from the N-terminus of the influenza HA-2 domain that enhances endosome escape. QD/peptide conjugates were prepared in which QDs were conjugated either with F3, with INF7, with both F3 and INF7, or with the random control peptide (KAREC). Cells were treated with each preparation and analyzed for QD internalization by flow cytometry. As shown in FIG. 11 (right panel), the greatest internalization was achieved using QDs conjugated with either F3 alone or F3 and INF7, thereby demonstrating the ability of F3 to enhance QD uptake. In another experiment, QDs are conjugated with an siRNA, and the ability of the various conjugates to silence expression of a target gene is assessed.

### Example 8

### Optimization of Targeted QD/siRNA Conjugates

### Materials and Methods

Materials

Quantum dots with emission maxima of 655 nm or 705 nm and modified with PEG and amino groups were obtained from Quantum Dot Corporation (ITK amino). QD concentrations were measured by optical absorbance at 595 nm, using extinction coefficients provided by the supplier. Cross-linkers used were sulfo-LC-SPDP (sulfosuccinimidyl 6-(3′-[2-pyridyldithio]-propionamido)hexanoate) (Pierce) and sulfo-SMCC (sulfosuccinimidyl-4-(N-maleimidomethyl)cyclohexane-1-carboxylate) (Sigma). Synthetic RNA duplexes directed against the EGFP mRNA were synthesized, with the sense strand modified to contain a 5′ thiol group (Dharmacon) (Sense: 5′-Th-(CH₂)₆-GGC UAC GUC CAG GAG CGC ACC, SEQ ID NO: 15; Antisense: 5′-UGC GCU CCU GGA CGU AGC CUU, SEQ ID NO: 16). The F3 peptide was synthesized with an animohexanoic acid (Ahx) spacer and cysteine residue added for conjugation (Final sequence: C[Ahx]AKVK DEPQR RSARL SAKPA PPKPE PKPKK APAKK; SEQ ID NO: 17). A FITC-labeled F3 peptide was also synthesized, along with KAREC (Lys-Ala-Arg-Glu-Cys; SEQ ID NO: 12), a five amino acid control peptide. All peptides were synthesized by N-(9-fluorenylmethoxycarbonyl)-L-amino acid chemistry with a solid-phase synthesizer and purified by HPLC. The composition of the peptides was confirmed by MS.

Conjugation of Peptides and Nucleic Acid to QDs

Amino-modified QDs were conjugated to thiol-containing siRNA and peptides using sulfo-LC-SPDP and sulfo-SMCC cross-linkers. QDs were resuspended in 50 mM sodium phosphate, 150 mM sodium chloride, pH 7.2, using Amicon Ultra-4 (100 kDa cutoff) filters. Cross-linker (1000-fold excess) was added to QDs and allowed to react for 1 hour. Samples were filtered on a NAP-5 gravity column (to remove excess cross-linker) into similar buffer supplemented with 10 mM EDTA. siRNA was treated with 0.1 M DTT for one hour and filtered on a NAP-5 column into EDTA-containing buffer. Peptides were typically used from lyophilized powder. Peptide and/or siRNA was added to filtered QDs and allowed to react overnight at 4° C. Using three Amicon filters, product was filtered twice with Dulbecco's phosphate buffered saline (PBS), twice with a high salt buffer (1.0 M sodium chloride, 100 mM sodium citrate, pH 7.2), and twice again with PBS. High salt washes were performed to remove electrostatically bound siRNA and peptide, which was not removed with PBS washes alone.

For siRNA-QDs, a 10-fold excess of siRNA was typically used for both cross-linkers. In the case of sulfo-LC-SPDP, the amount of conjugated siRNA was assayed using gel electrophoresis (20% TBE gel, Invitrogen), staining with SYBR Gold (Invitrogen). To confirm that similar amounts of siRNA (approximately 2 per QD) were conjugated to QDs using sulfo-SMCC, particles were stained with SYBR Gold and measured with a fluorimeter (SpectraMax Gemini XS, Molecular Devices).

For F3/siRNA-QDs and KAREC/siRNA-QDs, a molar ratio of 15:70:1 (siRNA:peptide:QDs) was found to be optimum, though a variety of ratios were attempted (FIG. 4A). These conditions yielded approximately 20 F3 peptides and 1 siRNA duplex per particle.

Cell Culture

Internalization and knockdown experiments were performed using a HeLa cell line stably transfected with 1 hour destabilized EGFP (courtesy of Phillip Sharp, MIT). Growth media was Dulbecco's modified Eagle's medium (DMEM) containing 4.5 g/l glucose and supplemented with 10% FBS, 100 units/ml penicillin, 100 μg/ml streptomycin, and 292 μg/ml L-glutamine. Cells were passaged into 24-well plates and used at 50%-80% confluency for internalization experiments and 20%-40% confluency for knockdown experiments.

For internalization experiments (FIG. 12), QDs were added to cell monolayers in media without serum at a final concentration of 50 nM. After four hours, cells were washed with media, treated with trypsin (0.25%) and EDTA, and resuspended in 1% BSA (in PBS) for flow cytometry (BD FACSort, FL1 for EGFP signal and FL3 for QD signal). Fluorescence data on 10,000 cells were collected for each sample and the geometric mean of intensity was reported.

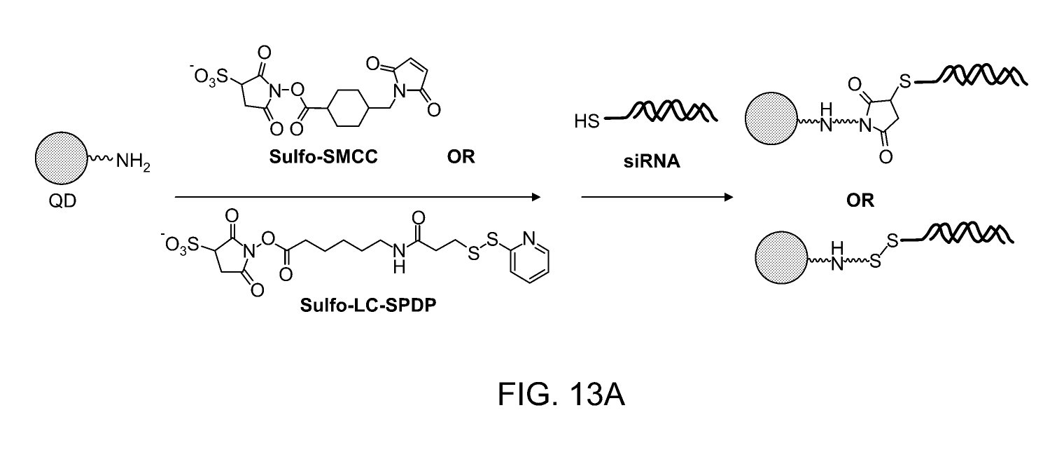

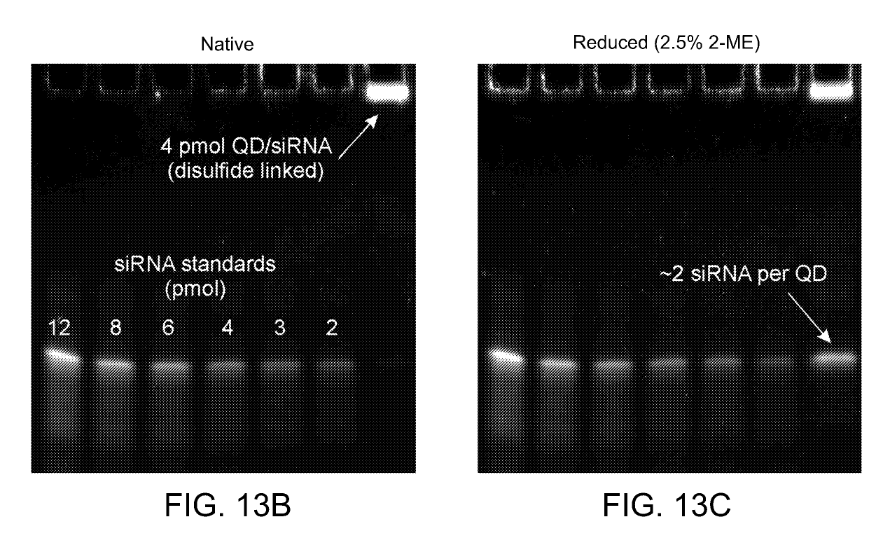

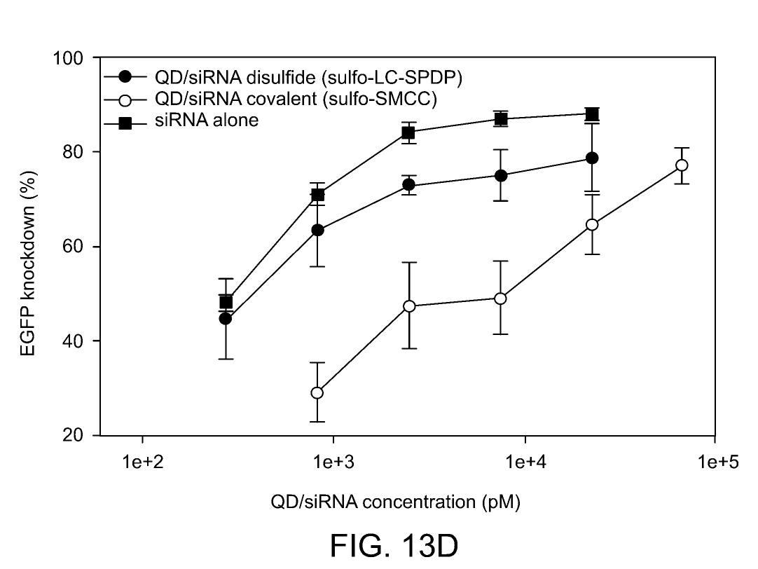

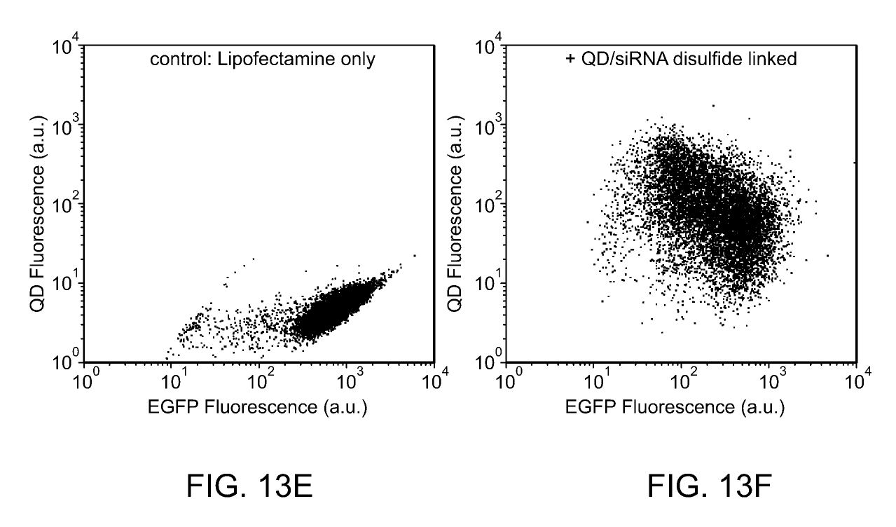

For knockdown experiments (FIG. 13), siRNA-QDs (in 50 μl serum/antibiotic-free media) were added to Lipofectamine 2000 (1 μl in 50 μl media, Invitrogen) and allowed to complex for 20 minutes. Cell media was changed to 400 μl of serum/antibiotic-free per well, and QD solutions (100 μl) were added dropwise. Complete media was added 12 hours-18 hours later. 48 hours after the QD were added, cells were trypsinized and assayed for fluorescence by flow cytometry.

To assess EGFP knockdown, 50 nM or 10 nM concentrations of F3/siRNA-QDs or KAREC/siRNA-QDs were added to cell monolayers (20%-40% confluent) in media with serum/antibiotics. Four hours later, cells were washed with similar media. Some samples were then treated with 1 μl of Lipofectamine per well (added dropwise in 100 μl media) either immediately after washing or after a 90 minute incubation at 37° C. (to allow membrane recycling). For all samples, media was changed to complete DMEM with serum/antibiotics approximately 16 hours after the addition of QDs, and assayed by flow cytometry 48 hours from the start of the experiment. For imaging, cells were initially seeded on glass-bottom dishes (Mat-Tek) and observed 48 hours after the addition of QDs using a 60× oil immersion objective. Images were captured with a SPOT camera mounted on a Nikon TE200 inverted epifluorescence microscope.

### Results

Taking a modular approach, particle internalization and siRNA attachment were investigated separately before these functions were combined in a single particle. First, peptides were conjugated to QDs to improve tumor cell uptake. Addition of as-purchased PEGlyated QDs to HeLa cell monolayers led to minimal cell uptake, as quantified with flow cytometry (FIG. 12A). Conjugation of siRNA or a control pentapeptide (KAREC) did not increase QD internalization, but addition of F3 peptide to the QDs improved the uptake significantly (two orders of magnitude). To confirm the specificity of F3 uptake, free F3 peptide was added to cells along with 50 nM F3-QDs (FIG. 12B). Dose-dependent inhibition of uptake was observed with F3 peptide concentrations from 1 μM to 1 mM. Inhibition of uptake by free KAREC peptide was minimal by comparison. The large excess of free peptide required for inhibition may be due to multiple copies of the F3 peptide on each QD and improved receptor binding as a result of multivalency.

To quantify the number of peptides added per particle, FITC-labeled F3 peptide was synthesized and attached to QDs using a cleavable cross-linker (sulfo-LC-SPDP). After filtering to remove unreacted peptide, 2-mercaptoethanol (2-ME) was added to reduce the disulfide bond between peptide and QD. Using a 100 kDa cutoff filter, F3-FITC peptide was separated from the QDs and quantified by fluorescence. Several reactions were performed with various amounts of FITC-F3 and siRNA as reactants. For each formulation, the cellular uptake was quantified by flow cytometry and F3 number measured (FIG. 12C, each point indicates a separate formulation). The results suggest that up to approximately 25 F3 peptides can be added per QD. Attachment of a small number of peptides (0-5) did not lead to significant uptake (less than 10% of maximum). Uptake increased with peptide number, but began to saturate around 15 copies per QD.

The use of cleavable (sulfo-LC-SPDP) or non-cleavable (sulfo-SMCC) cross-linkers for the attachment of F3 peptide did not significantly affect cell uptake. The choice of cross-linker, however, may affect the ability of the siRNA cargo to interact with RISC. The interior of the cell is a reducing environment, which would lead to cleavage of the disulfide bond generated by sulfo-LC-SPDP, freeing the siRNA. On the other hand, the amide bond produced by sulfo-SMCC is unaffected by reducing conditions (confirmed by treating the conjugates with 2.5% 2-ME for 30 minutes), leaving the intracellular QD/siRNA conjugate intact. We compared the efficiency of QD/siRNA conjugates prepared with both cross-linkers using an EGFP model system. Delivery of the conjugates to EGFP-labeled HeLa cells was performed by first complexing the particles with a cationic liposome transfection reagent (Lipofectamine 2000), to satisfy the functions of cell internalization and endosome escape, and knockdown efficiency was quantified by a reduction in EGFP fluorescence over controls (Lipofectamine only).

Using gel electrophoresis, the amount of siRNA conjugated per particle was quantified relative to double-stranded RNA standards. Particles conjugated using sulfo-LC-SPDP were first introduced under native (non-reduced) conditions (FIG. 13B). The absence of a siRNA band in the QD/siRNA lanes indicates that no siRNA is electrostatically bound to the particles. Exposing the particles to 2-ME for 30 minutes led to the appearance of a siRNA band in the SPDP lane, which can be quantified with RNA standards and ImageQuant software (FIG. 13C). Using this approach, approximately two siRNA duplexes were conjugated per QD under these conditions. Cellular fluorescence was quantified 48 hours after incubation with HeLa cells using flow cytometry. As hypothesized, the QD/siRNA formulation produced with the disulfide bond (using sulfo-LC-SPDP) led to greater EGFP knockdown (FIG. 3D).

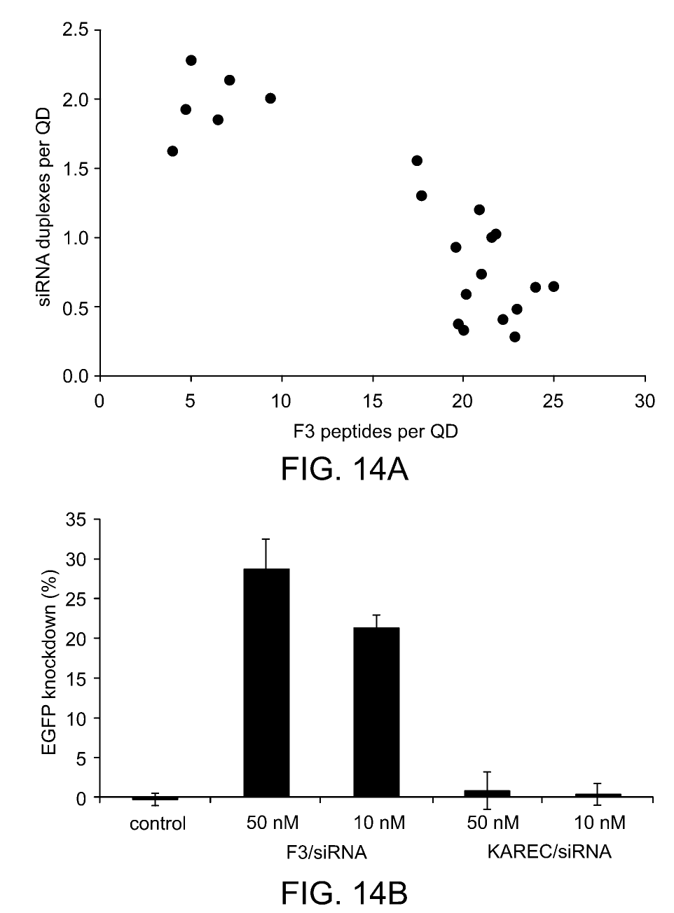

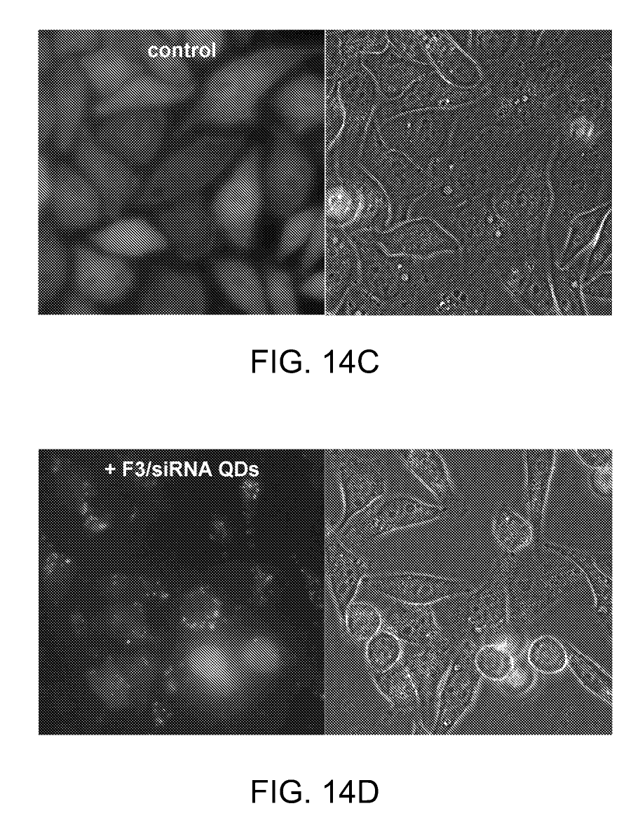

In addition to improved siRNA function, the use of a cleavable cross-linker allows the removal and quantification of both species after F3 peptide and siRNA co-attachment. The F3:siRNA reaction ratio was varied with the goal of generating a formulation capable of high cell uptake as well as the ability to carry a significant payload of siRNA. The results indicate a trade-off between one siRNA per particle with high uptake (>15 peptides) and two duplexes but low uptake (<10 peptides) (FIG. 14A). Negatively-charged siRNA may be electrostatically adsorbing to the surface of the aminated QDs, preventing the attachment of additional F3 peptides. Potentially, performing the reaction in high salt conditions, or in the presence of a surfactant, may allow higher loading. Since both high uptake efficiency and siRNA number are required for knockdown, particles with approximately 20 F3s and a single siRNA duplex were further investigated.

When incubated with cells, these particles were shown to internalize significantly, but did not lead to reduction in EGFP fluorescence 48 hours later. Fluorescence microscopy revealed that the particles were intracellular, but they colocalized with an endosomal marker (LysoSensor, Molecular Probes). Addition of an endosome escape agent, therefore, was used to achieve knockdown. Specifically, after incubation of cells with F3/siRNA-QDs and washing, cationic liposomes were added for 12 hours. Although cationic liposomes and polymers are typically used to form complexes with nucleic acids or particles, thereby ferrying the payload inside cells, in this case the reagent led to endosomal escape of previously internalized QDs. Without wishing to be bound by any theory, the cationic liposomes may be internalized into new endosomes, which fuse with the endosomes carrying the QDs. As the pH of the vesicle is lowered by the cell, osmotic lysis leads to the release of both species into the cytoplasm. To assess the importance of the targeting ligand, particles carrying siRNA and a control peptide (KAREC) were used. These KAREC/siRNA particles were not internalized, and no EGFP knockdown was observed, despite endosome disruption. Additionally, a time lag of 90 minutes between washing the cells free of QDs and cationic liposome addition did not lead to significant reduction in efficiency, indicating that endosomal degradation of the siRNA is not an issue on this time scale.

In addition to cationic liposomes, some chemotherapeutics, such as chloroquine have been shown to result in endosomal escape (Won et al., 2005, Science, 309:121; incorporated herein by reference). While an endosome escape step could be a realistic part of a treatment regimen, there is also potential that this function could be built into each particle. Addition of a fusogenic peptide to the QD surface, for example, may further improve delivery of the multifunctional particles described (Plank et al., 1994, J. Biol. Chem., 269:12918; incorporated herein by reference).

Decorating the surface of a fluorescent quantum dot with both a targeting ligand and siRNA duplex requires a tradeoff in the number of each species but can be used to generate a conjugate capable of knockdown in vitro. We found that multiple copies of the F3 targeting peptide were required for QD uptake, but that siRNA cargo could be co-attached without affecting the function of the peptide. Disulfide (sulfo-LC-SPDP) and covalent (sulfo-SMCC) cross-linkers were investigated for the attachment of siRNA to the particle, with the disulfide bond showing greater silencing efficiency. Finally, after delivery to cells and release from their endosomal entrapment, F3/siRNA-QDs led to knockdown of EGFP signal. By designing the siRNA sequence against a therapeutic target (e.g. an oncogene) instead of EGFP, this technology may be adapted to treat and image metastatic cancer. The technology explored in this study could be readily adapted to other nanoparticle platforms, such as iron oxide or gold cores, which allow image contrast on magnetic resonance or x-ray imaging respectively and may therefore mitigate concerns over QD cytotoxicity and the limited tissue penetration of light. QDs, however, remain an attractive tool for in vitro and animal testing, where fluorescence is the most accessible and common imaging modality.

### Example 9

### Photoactivation of Endosomal Escape

### Materials and Methods

Fluorescein labeled CAR peptide (CARSKNKDC) was synthesized using an ABI Model 433A peptide synthesizer from Biopolymers Laboratory, the MIT Center for Cancer Research. The peptide was cyclized by bubbling air into an aqueous solution of the peptide at 0.1 mg/ml overnight. Complete cyclization was confirmed by mass spectrometry and HPLC analysis.

Glioblastoma cells were obtained from the laboratory of Phil Sharp (MIT). The cyclized peptide (cCAR) was incubated with cells for 2 hours at 37° C. in complete culture medium (DMEM supplemented with serum, streptomycin, penicillin and fungizone). A monolayer of the cells was then rinsed with warm media three times. Microscopy photographs were taken after overnight incubation of the cells. For activation of photosensitizer, Arc lamp light from a microscope was irradiated onto the cells for two minutes.

### Results

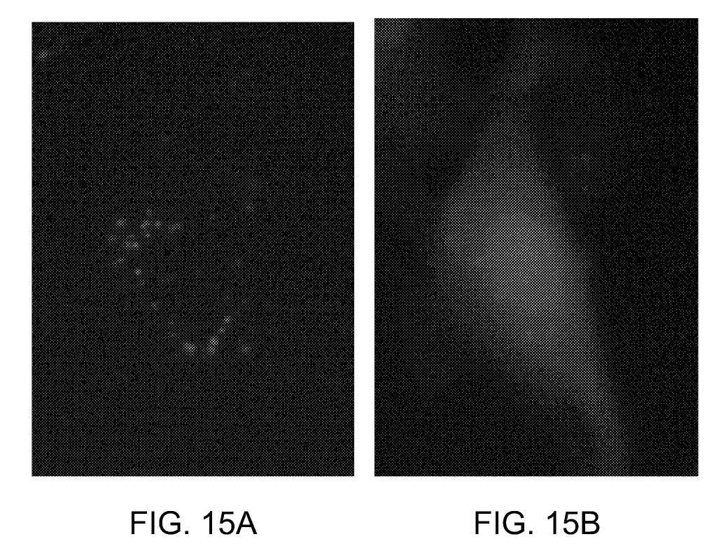

The cyclic CAR peptide was internalized into glioblastoma cells. Without light irradiation, bright, punctate fluorescent spots inside cells were observed, which likely represent peptides that are trapped in endosomes (FIG. 15). Upon light irradiation, bursting of the green fluorescent spots was observed (FIG. 15). Eventually, green fluorescence became distributed evenly inside cells followed by rapid nuclear localization of the peptide (FIG. 15). The result shows that a photosensitizer can effectively induce endosomal escape upon light irradiation.

### Example 10

### siRNA and Targeting Peptide are Conjugated to Nanoparticles Via Protease-Cleavable Peptide

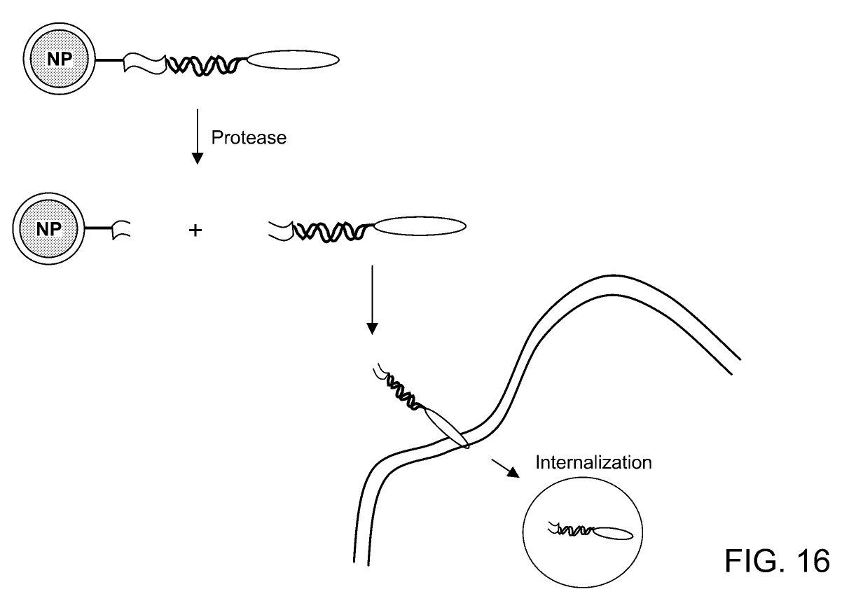

In certain embodiments, nanoparticles are associated with one or more entities that mediate controlled release of an agent. In some embodiments, an agent and targeting peptide are conjugated to nanoparticles via protease-cleavable peptides. Cleavage will occur the sites where corresponding proteases are present. Proteases such as matrix metalloproteases (MMPs) are upregulated in many types of tumors. Therefore, agents to be delivered that are conjugated to nanoparticle entities via protease-cleavable bonds are released from nanoparticles when nanoparticles reach tumor sites in vivo. Results of such an experiment are presented in FIG. 16.

### Example 11

### Multifunctional Nanoparticles are Multivalent and can be Remotely Actuated and Imaged Noninvasively In Vivo

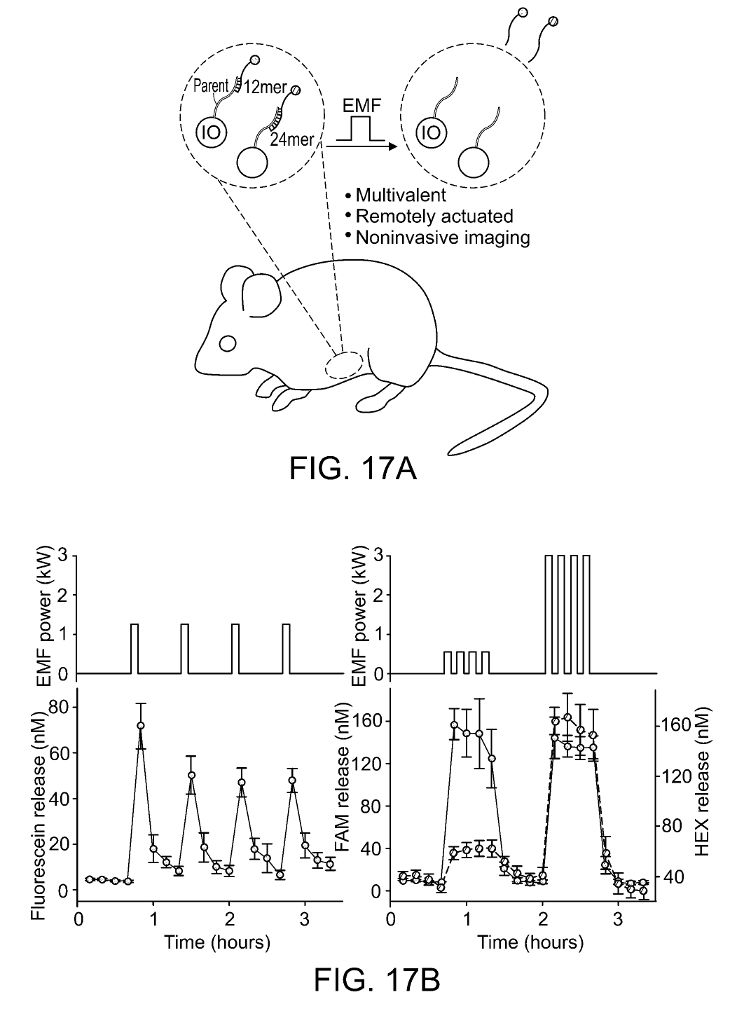

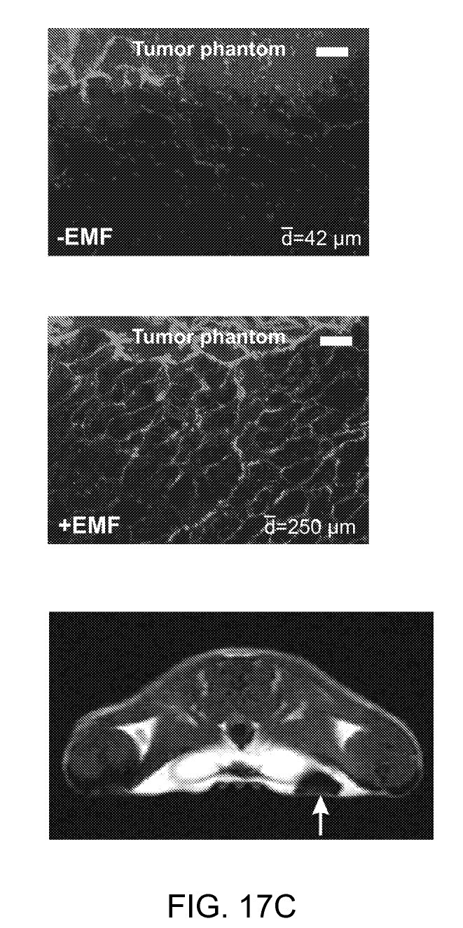

Superparamagnetic nanoparticles of 50 nm act as transducers to capture external electromagnetic energy not absorbed by tissue (350 kHz-400 kHz) to break bonds on demand (FIG. 17A). Use of a nucleic acid strand conjugated to the nanoparticle and a model drug attached to its complement formed a tunable, heat-labile linker. The multifunctional nanoparticles were used to demonstrate remote, pulsatile release of a single species and complex, multistage release of two species from their surface in vitro, and further used for noninvasive imaging and remote actuation upon implantation in vivo.

Pulsatile release of a fluorophore by electromagnetic field (EMF) pulses (400 kHz, 1.25 kW) of 5 minute duration every 40 minutes was performed (FIG. 17B). Such a profile would be useful for metronomic dosing of a cytotoxic or cystostatic drug. The use of nucleic acid duplexes as a heat-labile linker has the additional feature of temperature tunability through changes in chain length and variations in G/C content. Oligonucleotides of two different lengths and corresponding fluorescent species (12mer, FAM, 24mer, HEX) were used to demonstrate the potential for complex release profiles (FIG. 17B). Low power EMF pulses (0.55 kW) trigger release predominantly of FAM by melting of the 12mer whereas higher power (3 kW) led to simultaneous release of both species. Such a profile could be used to release multiple drugs in series, synergistic drug combinations such as a chemosensitizer and chemotherapeutic, or combination regimens such as antiangiogenic and cytotoxic compounds (Boutros et al., 2004, Science, 303:832; incorporated herein by reference).

To explore the use of the multifunctional nanoparticles in vivo, a subcutaneous tumor phantom was implanted consisting of a matrigel plug containing nanoparticles in living mice. The release of a model drug was examined by EMF exposure of 3 kW and 5 minutes. Fluorescent micrographs of histological sections in FIG. 17C depict an increase in penetration depth of the model cargo into surrounding tissue due to EMF exposure by approximately six-fold over unexposed controls. Such an increase in penetration depth could prove useful for treatment of peripheral disease—areas often underdosed in hyperthermia generated by thermal seeds. The use of the particle core to transduce external EMF energy to break local bonds is an advantage over near-infrared light and other potential remote triggers that are more efficiently absorbed by tissue (Zheng et al., 2004, Proc. Natl. Acad. Sci. USA., 101: 135; incorporated herein by reference).

FIG. 17D depicts the noninvasive visualization of the nanoparticles by magnetic resonance imaging, demonstrating the potential utility as both diagnostic and therapeutic vehicles.

The strategy outlined here serves merely as a starting point for the fabrication of integrated, multifunctional nanodevices that offer the potential to shift the current paradigm whereby diagnostics and therapeutics are sequential elements of patient care. In this example, nanoparticles could be delivered intravascularly using homing peptides (Akerman et al., 2002, Proc Natl Acad Sci USA, 99:12617; incorporated herein by reference), used to visualize diseased tissue by MRI, and used to guide focused application of electromagnetic energy, ultimately enabling remote, physician-directed drug delivery with minimal collateral tissue exposure. Clearly, the performance of these devices can be improved in the future by new materials (particle cores, heat-labile tethers, small molecule drugs, targeting species) and approaches to their effective integration.

### Example 12

### siRNA Degradation by Serum Can Be Reduced by Co-Immobilization with PEG

### Materials and Methods

Thiolated siRNAs were purchased from Dharmacon (Lafayette, Colo.). Other reagents were obtained from Sigma-Aldrich (St. Louis, Mo.).

Gold nanoparticles were synthesized according to literature (Frens, 1973, Nature, 241:20; incorporated herein by reference). Thiolated siRNA with or without PEG (5 kDa) were conjugated to the prepared nanoparticles by mixing with prepared gold colloids and incubate the solution overnight at room temperature. Functionalized gold colloids were purified by repeated centrifugation and resuspension of the colloidal pellets in doubly distilled water. Degradation kinetics of siRNA (or siRNA-colloid conjugates) were performed in 50% mouse serum at 37° C. Amount of siRNAs at different time points was measured by gel electrophoresis.

### Results

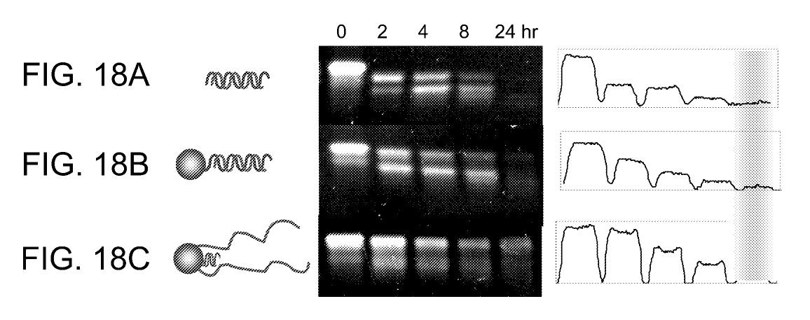

Addition of PEG to nanoparticle-siRNA formulation increased the stability of siRNA in serum. Compared to free siRNAs or siRNA-colloid without PEG, degradation of colloid-conjugated siRNAs along with PEG was slower (FIG. 18; approximately 40% of original siRNAs vs. approximately 1% after 24 hour incubation).

### Example 13

### Protease-Triggered Unveiling of Bioactive Nanoparticles

The modification of nanomaterials with biological recognition motifs enables a myriad of functions that have been exploited for cancer diagnostics and therapeutics. While bioactive domains can be used to target nanoparticles to cell receptors, shuttle them across cell membranes, or activate cell signaling, these motifs typically employ cationic or hydrophobic regions that lead to rapid mononuclear phagocytic system (MPS) clearance of particles from the blood, ultimately reducing particle accumulation in the tumor (Moghimi et al., 2001, Pharmacol. Rev., 53:283; and Weissleder et al., 1995, Adv. Drug Deliv. Rev., 16:321; both of which are incorporated herein by reference). Further functionalization with hydrophilic polymers such as polyethylene glycol (PEG) can improve blood half-lives and tumor accumulation, but also introduces an entropic penalty that inhibits ligand-mediated nanoparticle function (Alexander, 1977, J. de Physique, 38:983; Degennes, 1980, Macromolecules, 13:1069; and Storm et al., 1995, Adv. Drug Deliv. Rev., 17:31; all of which are incorporated herein by reference). To address this apparent paradox between improved biodistribution and optimal functionality, the present inventors present a general strategy for veiling and unveiling bioactive domains on nanoparticles with sterically protective polymers, so that they passively accumulate in the hyperpermeable vasculature of tumors, but can be activated by cancer-secreted proteases to unveil hidden functional domains.

Previously, we demonstrated that veiling particles with protease-cleavable polymers effectively suppresses the binding of complementary small molecules and larger proteins on nanoparticles (Harris et al., 2006, Angewandte Chemie-Intl. Ed., 45:3161; and von Maltzahn et al., 2007, J. Am. Chem. Soc., 129:6064; both of which are incorporated herein by reference). In this work we extend the utility of this technique by demonstrating the favorable properties of these coatings in vivo. In contrast to the reported use of cleavable PEGs to destabilize and fuse liposomes (Hatakeyama et al., 2007, Gene Therapy, 14:68; and Zhang et al., 2004, Pharm. Res., 49:185; both of which are incorporated herein by reference) or the use of cleavable polyanionic peptides to electrostatically neutralize cationic domains (Jiang et al., 2004, Proc. Natl. Acad. Sci., USA, 101:17867; and Zhang et al., 2006, Nano Letters, 6:1988; both of which are incorporated herein by reference), this strategy exploits the entropic penalty imparted by hydrophilic polymers on approaching surfaces to veil and unveil the bioactivity of surface ligands. Consequently, this technique may be used to veil bioactive domains that mediate a variety of functions besides fusion or internalization, such as cell-binding or cell signaling, and need not be cationic or lipid-like.

### Materials and Methods

Unless otherwise stated all reagents were purchased from Sigma-Aldrich and all reactions were performed at room temperature.

Synthesis of Nanoparticles

The nanoparticles used in these experiments were synthesized, cross-linked, aminated, and labeled with a near-infrared fluorophore (VivoTag 680) according to published protocols (Josephson et al, 1999, Bioconj. Chem., 10:186; incorporated herein by reference). To conjugate species onto nanoparticles, surface amines were functionalized with SIA (N-succinimidyl iodoacetate) to make them thiol reactive. A FITC-labeled poly-arginine cell internalizing peptide, NH₂—RRRRGRRRRK(FITC)GC (SEQ ID NO: 18), and a TAMRA-labeled protease-cleavable PEG, prepared by coupling the amine terminus of an MMP-2 cleavable peptide substrate, NH₂-GK(TAMRA)GPLGVRGC (SEQ ID NO: 19), to 10 kD NHS-PEG (von Maltzahn et al, 2007, J. Am. Chem. Soc., 129:6064; incorporated herein by reference), were then linked to nanoparticles via thiol groups on the cysteine residues at the carboxyl termini. A more detailed protocol is available in the supplemental section.

Superparamagnetic iron oxide nanoparticles were synthesized according to published protocols (Palmacci and Josephson, 1993, U.S. Patent Vol. 5, p. 176). Briefly, dextran-coated iron oxide nanoparticles were synthesized, purified, and subsequently cross-linked using epichlorohydrin. After exhaustive dialysis, particles were aminated by adding 1:5 v/v ammonium hydroxide (30%) and incubated on a shaker overnight. Aminated-nanoparticles were subsequently purified from excess ammonia using a Sephadex G-50 column and concentrated using a high-gradient magnetic-field filtration column (Miltenyi Biotec, Auburn, Calif.). Amine functionalized particles were labeled with the NHS ester NIR fluorochrome, VivoTag 680 (VisEn Medical, Woburn, Mass.), by adding 1:20 w/w and incubating on a shaker for one hour. Excess dye was removed by filtration on a Sephadex G-50 column. The particle molarity was determined by the viscosity/light scattering method (Reynolds et al., 2005, Analytical Chem., 77:814; incorporated herein by reference).

Peptide-PEG Synthesis

Peptides were synthesized in the MIT Biopolymers core to contain sequentially, an amino terminus for PEG attachment, a TAMRA-labeled lysine, an MMP-cleavage sequence, and a cysteine at the carboxy terminus for particle attachment. The purity of the cleavable MMP2 substrate (NH₂-G-K(TAMRA)-G-P-L-G-V-R-G-C—CONH₂; SEQ ID NO: 19) and the uncleavable D amino acid analogue (NH₂-G-K(TAMRA)-G-dP-dL-G-dV-dR-G-C—CONH₂; SEQ ID NO: 20) was verified with HPLC and mass spectrometry. Amine-reactive 10 kDa mPEG-SMB reagents (methoxy-polyethylene glycol-succinimidyl α methylbutanoate) were purchased from Nektar Therapeutics. Peptides were reacted with polymers in PBS+0.005 M EDTA pH 7.2 at 500 μM and 400 μM, respectively, for >24 hours with shaking. Free peptide was removed by reducing with 0.1 M TCEP and filtered using a G-50 Sephadex column. Reduced polymer was then quantified using fluorochrome extinction and added to nanoparticle preparations as described below.

Ligand Attachment to Nanoparticles

Attachment of peptide-PEGs to nanoparticles was performed simultaneously with attachment of cell internalizing peptides (NH₂—RRRRGRRRRK(FITC)GC, SEQ ID NO: 18, MIT Biopolymers). The internalizing peptide purity was verified by HPLC and mass spectrometry and its concentration was quantified using the molar extinction coefficient of FITC. Aminated nanoparticles (1.3 mg Fe/ml) were reacted with N-succinimidyl iodoacetate (11 mM) in 0.1 M HEPES, 0.15 M NaCl pH 7.2 (HEPES buffer) for 3 hours and filtered using a G-50 Sephadex column into phosphate buffered saline+0.005 M EDTA pH 7.2 (PBS-EDTA buffer). Purified nanoparticles (0.06 mg Fe/ml) were then combined with stock solutions of reduced peptide-PEG (60 μM) in PBS-EDTA buffer and internalizing peptide (serial dilutions of 63 μM, 50.4 μM, 37.8 μM, 25.2 μM, 12.6 μM, & 0 μM) in 0.1% TFA at 1:3 and 1:0.1 v/v respectively. The stock concentration selected for the optimized particle was 25.2 μM. The number of ligands per particle was determined spectrophotometrically using a pre-determined extinction coefficient for iron nanoparticles, FITC-labeled internalizing peptide, and TAMRA-labeled peptide PEG at 400 nm, 495 nm, and 555 nm respectively. The optimized particle was determined to have 16 VT 680 dyes, 6 internalizing peptides, and 60 peptide-PEGs.

Flow Cytometry

HT080 human fibrosarcoma cells (ATCC) cells were cultured in 24 well plates and grown to 80% confluency using ATCC recommended media. Veiled and MMP pre-cleaved nanoparticles (100 μl at 0.1 mg/ml Fe) were added to 400 μl cell culture media with 25 μM Galardin and incubated over cells for 1 hour. Adherent cells were detached from the tissue culture plate with 0.25% trypsin, washed in PBS, and analyzed on a Beckman Dickson LSR II using a 633 nm excitation source and a 690/40 band pass filter to detect VT-680 labeled nanoparticles in cells.

GLIO 1431 (obtained from Al Charest at Tuft's University), TRAMP (obtained from Jianzhu Chen at M.I.T.), and MDA-MB-435 (obtained form Erkki Ruoslahti at the Burnham Institute) were cultured in DMEM media with 10% FCS and 1% P/S and grown to 80% confluency. Veiled and MMP-activated (unveiled) nanoparticles (100 μl at 0.1 mg/ml Fe) were added to 400 μl cell culture media with 25 μM Galardin and incubated over cells for various times. For flow cytometry studies, adherent cells were detached from the tissue culture plate with 0.25% trypsin, washed in PBS, and analyzed on a Beckman Dickson LSR II using a 633 nm excitation source and a 690/40 band pass filter to detect VT-680 labeled nanoparticles in cells. Microscopy was conducted on live cells in glass bottom wells using a 100× objective and a cy5.5 filter cube (Chroma).

MMP Activation

Unless otherwise stated, pre-cleaved (unveiled) particles were prepared by incubating nanoparticles with 20 μg/ml collagenase (Clostridiopeptidase A) in 0.1 M HEPES 0.15 M NaCl pH 7.2 (HEPES buffer) with 5 mM CaCl₂. Activation was monitored by the release of TAMRA quenching at an excitation of 515 nm and emission of 580 nm. Addition of 25 μM of the broad-spectrum MMP inhibitor (Galardin) prevented cleavage of peptide-PEGs as monitored by dequenching (FIG. 25).

K_{cat}/Kₘ Determination

Cloaked nanoparticles (0.05 mg/ml Fe) coated with 2.9 μM of peptide-PEG substrate in HEPES buffer with 5 mM CaCl₂ were incubated with recombinant MMP-2 (0.724 μg/ml) and monitored fluorimetrically to assess activation. The Vₘₐₓ of fluorescence release of particles at this concentration was linearly related to that of particles at concentrations ½ and 2-fold as much indicating that the substrate concentration [S] was much less than the binding constant Kₘ in this experimental setup. Activation experiments were quenched by the addition of 0.1 M EDTA at 1:9 v/v. Particles were ultracentrifuged and the supernatant collected to measure product formation. Similarly free peptide ([S]=15.45 μM) in HEPES buffer+5 mM CaCl₂ was incubated with recombinant MMP-2 (0.3367 μg/ml). Activation was quenched by the addition of 0.1 M EDTA at a 1:9 v/v and cleavage was monitored using a fluorescamine assay. The Vₘₐₓ of substrate cleavage during the first 30 minutes for substrate concentrations of 15.45 μM and 7.75 μM were linearly related confirming that the experiment was operating in a range of [S] much less than Kₘ. The reaction was driven to completion over 24 hours and the change in fluorescamine signal at various time points was used to determine the substrate concentration.

Multimodal Imaging in Agarose Wells

A 5% agarose solution in water was boiled and then cooled in a cell culture dish containing well molds from centrifuge tubes. Each well was filled with 8 million cells from a 40% confluent T-150 flask. HT-1080 cells in these flasks were incubated with nanoparticles (1 μg/ml Fe) in DMEM with serum media for various times. Particles were removed after incubation and cells were trypsinized, washed in PBS, fixed overnight in 50 μl of PBS with 4% paraformaldehyde, and transferred to agarose wells for imaging. MRI images were taken on a Bruker 4.7 T magnet, 7 cm vore. A series of 32 images with multiples of 15 ms echo times and a TR of 3000 ms were acquired. T2 maps were obtained for each well using the T2 fit map plug-in in Osirix imaging software. A fluorescence scan through the wells was acquired on an Odyssey Infrared System (Licor) using the 700-emission channel to detect VT680 labeled particles.

Xenograft Animals

Nude mice were injected s.c. bilaterally in the hind flank with 2×10⁶ HT-1080 cells. After 1 week-2 weeks, animals were anaesthetized with isoflurane and injected through the tail vein with nanoparticles (4 mg/kg-10 mg/kg Fe). Animals were imaged before and 24 hours after intravenous injection of nanoparticles (10 mg/Kg Fe) on a 4.7 T Bruker magnet. A series of 16 images with multiples of 8.6 ms echo times and a TR of 2133.3 ms was acquired. T2 maps were obtained for regions of interest using the T2 fit map plug-in in OsiriX. At 48 hours, animals were imaged by a fluorescence molecular tomography (FMT) imaging system (Visen Medical). Quantitative analysis of relative nanoparticle uptake in tumors by FMT was assessed by selecting regions of interest around tumor masses 4 mm-6 mm in diameter. Quantitative measurements on dye concentrations were normalized by the total injected dose in each animal to yield relative fluorescent units (RFUs). Blood half-lives were determined by the decrease in fluorescence intensity of 25 μl blood samples withdrawn sub-orbitally with heparinized microcapillary tubes. Animals were euthanized by cervical dislocation under anesthesia and tumors were harvested, embedded in OCT, and stored at −80° C. for cryosectioning. Samples were cut into 5 μm sections using a cytochrome and fixed in cold acetone for staining and imaging.

Colocalization Analysis

Histological sections were labeled with an anti-TAMRA primary antibody (AbD Serotec) and an Alexa-750 secondary antibody (Invitrogen) to stain the presence of TAMRA-labeled polymer on nanoparticles in the tumor tissue. Twelve image fields from three different tumor specimens were acquired for animals injected with cleavable (L-AA) and uncleavable (D-AA) nanoparticles. To cancel the background signal from noise and non-specific antibody binding, the cumulative distribution of pixel intensity data from all analyzed fields was generated for VT-680 and TAMRA antibody channels, and a value determined from the inflection point was subtracted from all images. Mander's Coefficients, which represents particle colocalization with TAMRA-labeled internalizing domains, were computed for each image using the WCIF Mander's Coefficient plug-in for ImageJ.

### Results and Discussion

Using fluorescence imaging and MRI, we indeed demonstrate that protease-removable polymer coatings effectively shut down cell uptake of nanoparticles bearing cell internalization domains, while proteolytic cleavage by MMP-2, a protease upregulated in angiogenesis, invasion, and metastasis (Davidson et al., 1999, Gynecol. Oncol., 73:372; Fang et al., 2000, Proc. Natl. Acad. Sci., USA, 97:3884; Giannelli et al., 1997, Science, 277:225; and Stearns and Wang, 1993, Cancer Res., 53:878; all of which are incorporated herein by reference), restores internalization function. In vivo, reversible polymer veiling greatly extends nanoparticle circulation in the blood over unveiled particles and enhances accumulation in the tumor. We confirm that removable coatings on extravasated nanoparticles are removed in the tumor, thus establishing the potential of this design for unveiling bioactive ligands in response to disease-associated triggers on a variety of nanoparticle platforms.

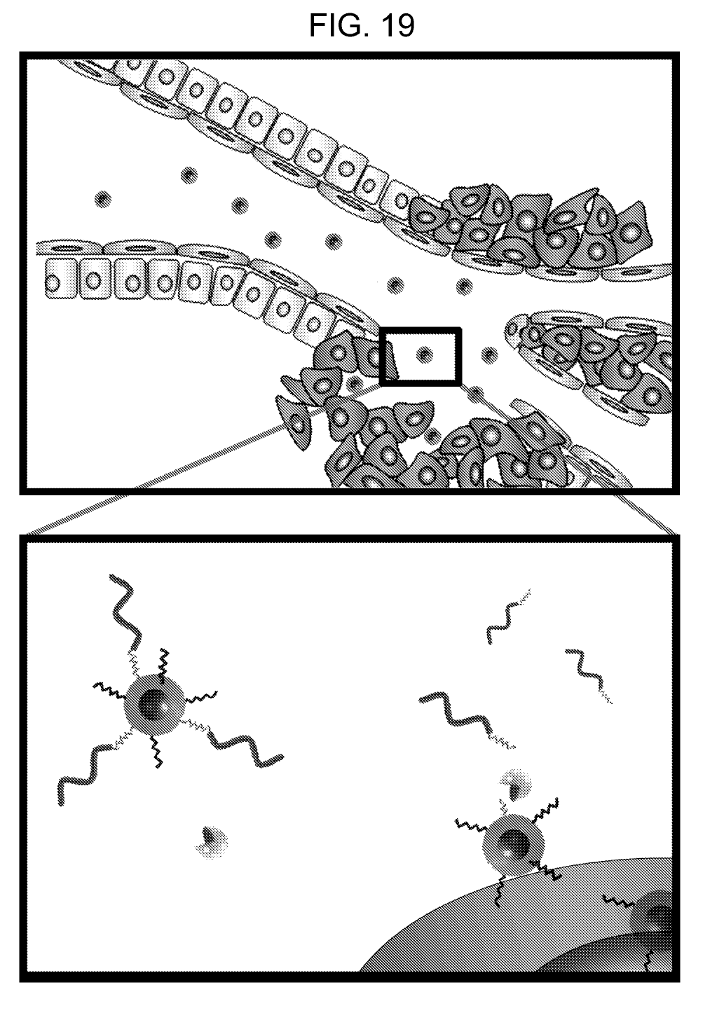

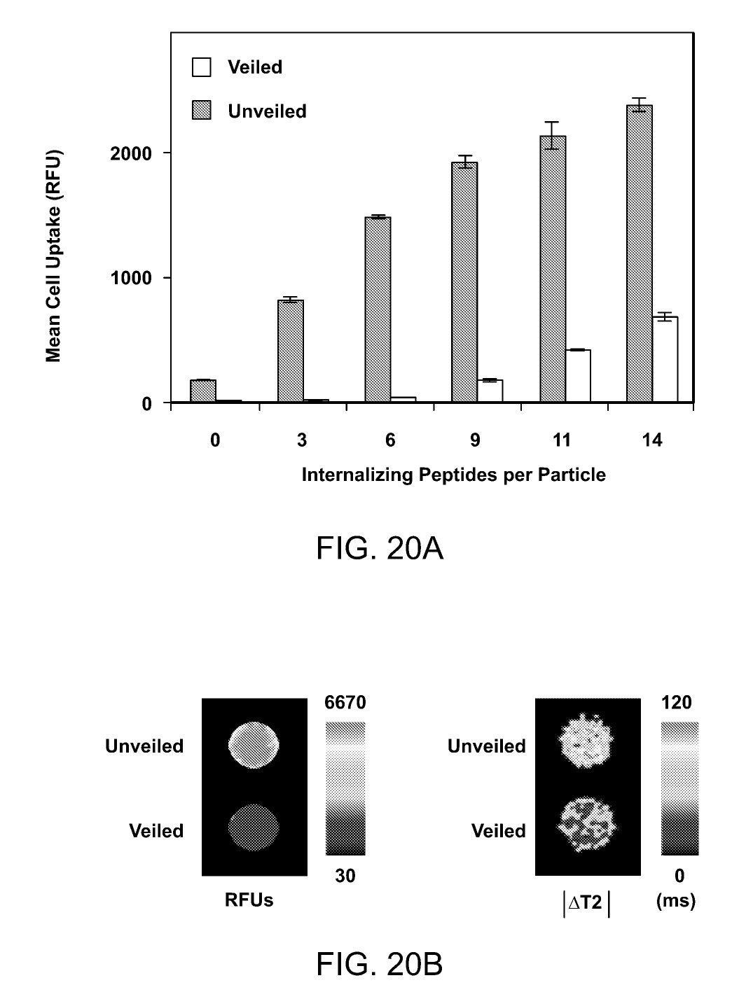

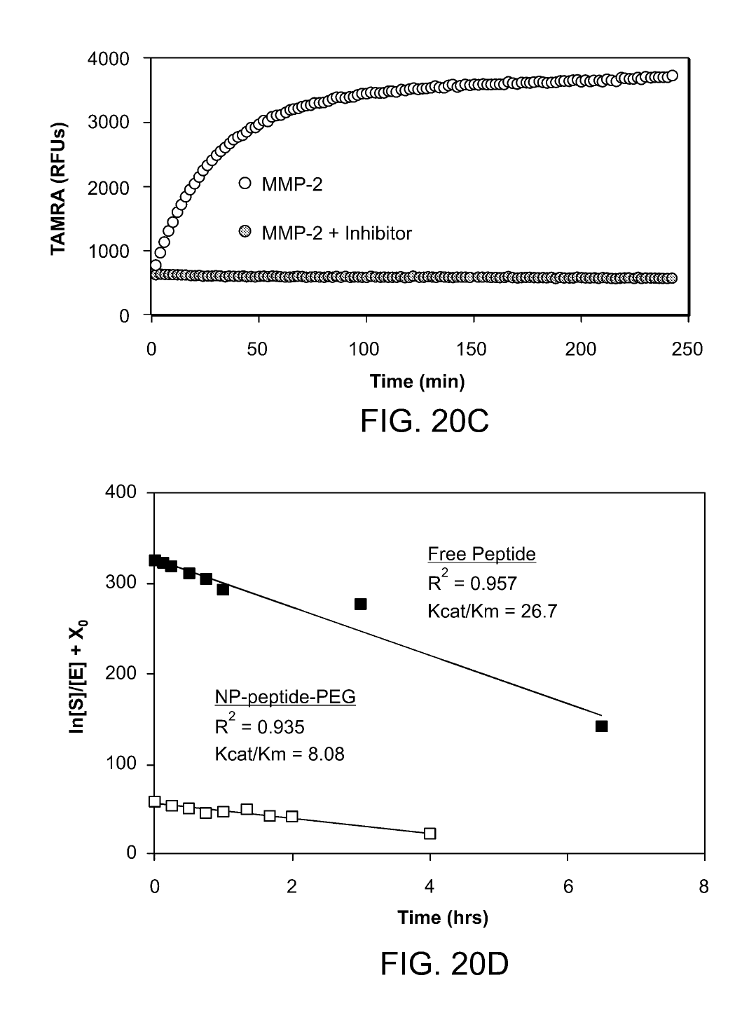

FIG. 19 shows a schematic model of nanoparticles bearing protease-removable polymer coatings that veil and unveil the function of bioactive surface ligands. Two species, a cell internalization domain and a removable hydrophilic polymer, consisting of a linear PEG tethered by an MMP-2 cleavable substrate are conjugated onto the surface of a magnetofluorescent dextran-coated iron oxide nanoparticle. Prior to activation, the hydrophilic polymer prevents: (1) adsorption of serum opsonins and MPS-mediated clearance of the particles, and (2) systemic action of the bioactive ligand, an internalizing domain. Previously we identified a removable polymer coating that was optimal for veiling and unveiling interparticle interactions (Harris et al., 2006, Angewandte Chemie-Intl. Ed., 45:3161; incorporated herein by reference). We hypothesized that this approach could be extended to veil and unveil particle-cell interactions. To test this, we conjugated particles with the removable polymer coating and varying densities of cell internalization domains and then measured the uptake of veiled and protease-activated (unveiled) particles by HT-1080 cells using flow cytometry. Particles with lower domain densities were taken up by cells minimally in both the veiled and unveiled state, while particles with higher domain densities were taken up by cells even with the polymer coating intact. An optimized particle design was selected based on a high level of internalization of unveiled particles and a low level of internalization of veiled particles, with the optimum ratio of internalization domains resulting in a 40-fold increase in cell accumulation (FIG. 20A). This particle had, on average, 6 internalization domains per nanoparticle. The unmodified particles were 65±5 nm by DLS and increased to 90±5 nm after applying the polymer coating.

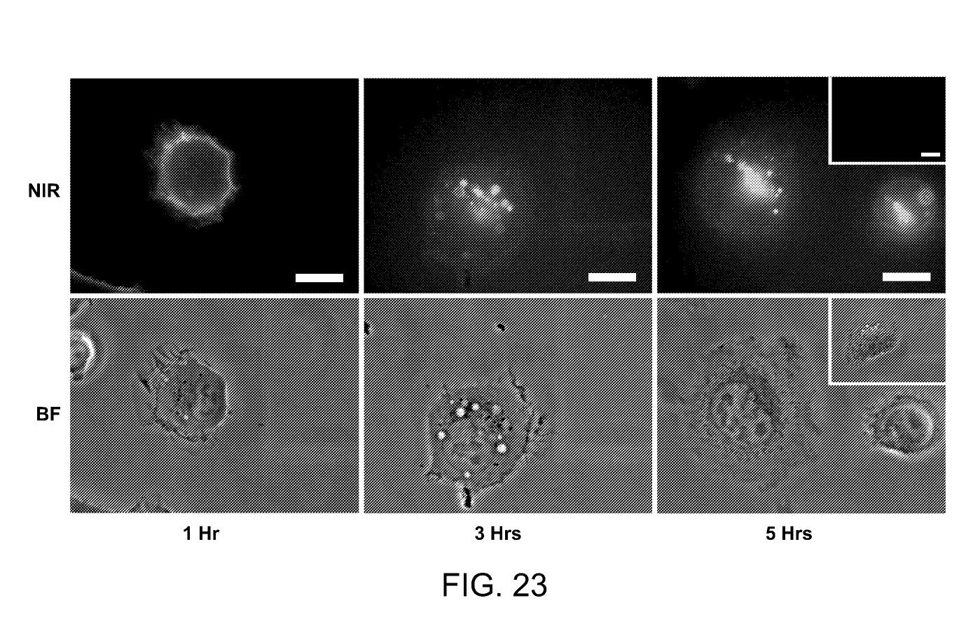

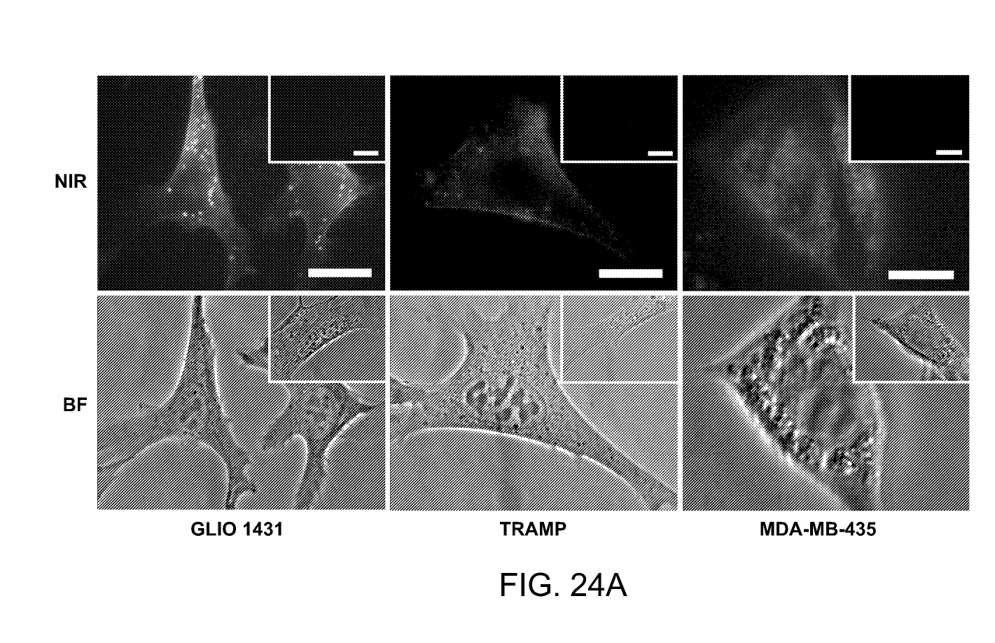

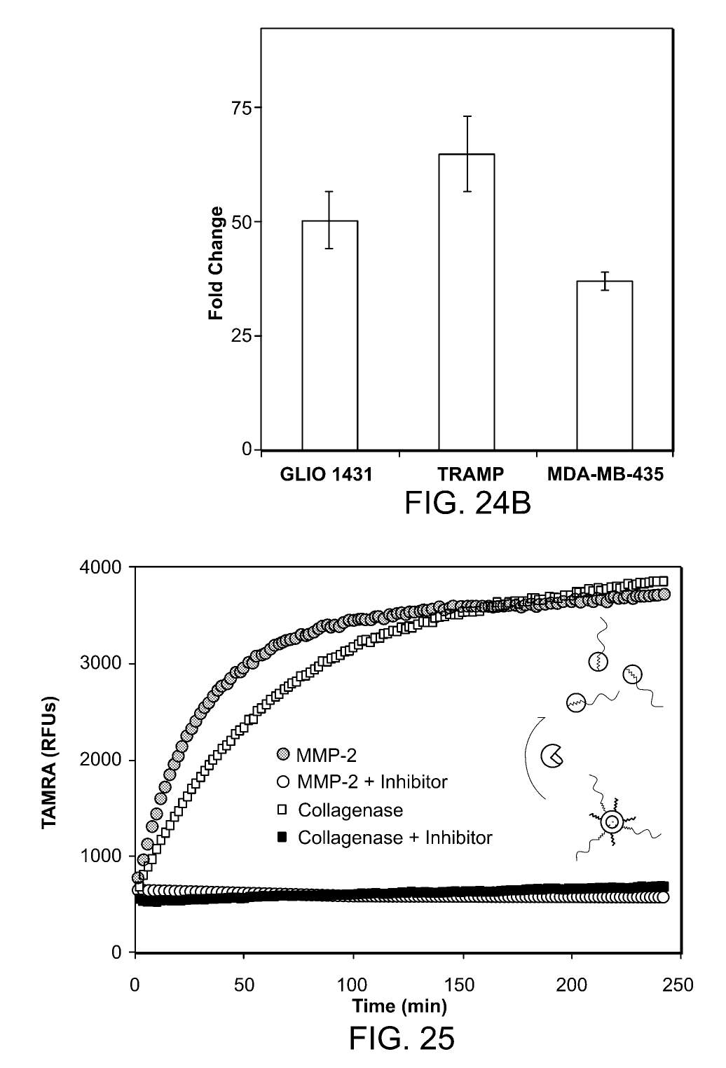

To verify that internalization function is indeed restored after removal of the polymer coating, epifluorescence microscopy was used to monitor the trafficking of unveiled nanoparticles as they traveled from the cell membrane to the nucleus through punctate intracellular organelles, a pattern greatly reduced with veiled particles (FIG. 23). Flow cytometry and microscopy studies using other cell lines confirmed that this effect is not specific to HT-1080 cells only (FIG. 24). The magnetic properties of the iron-oxide core particles used in these studies can also be used to confirm cell uptake of the particles with MRI. A T2 mapping sequence was used to detect T2 changes in cells that had been incubated with veiled and unveiled nanoparticles for 5 hours and imaged by a 4.7 T MRI (Bruker). The internalization of nanocrystal cores leads to a measurable decrease in T2 signal which was significantly greater with unveiled particles and well correlated with signal changes detected in a planar fluorescence scan (Licor —FIG. 20B).

Given the complex orientation of the cleavable substrate in this scheme, we sought to evaluate the kinetics of nanoparticle activation by deriving the catalytic rate (K_{cat}) over the binding constant (Kₘ) for MMP-2 and its substrate when immobilized between a particle and PEG versus free in solution. Since peptide-PEG domains were labeled with TAMRA in a position that would be removed upon cleavage, activation of nanoparticles by MMP-2 relieves TAMRA-iron quenching interactions and consequently increases TAMRA fluorescence several fold, enabling real-time monitoring of activation in solution and determination of the K_{cat}/Kₘ (FIGS. 20C,D). Using this approach, it was determined that PEG shielding and particle immobilization of the peptide contributed to a 3.2-fold decrease in its associated MMP-2 K_{cat}/Kₘ, a favorable reduction considering the order of magnitude decreases that have been reported with MMP-2 substrates on other immobilized polymers (Chau et al., 2004, Bioconj. Chem., 15:931; incorporated herein by reference).

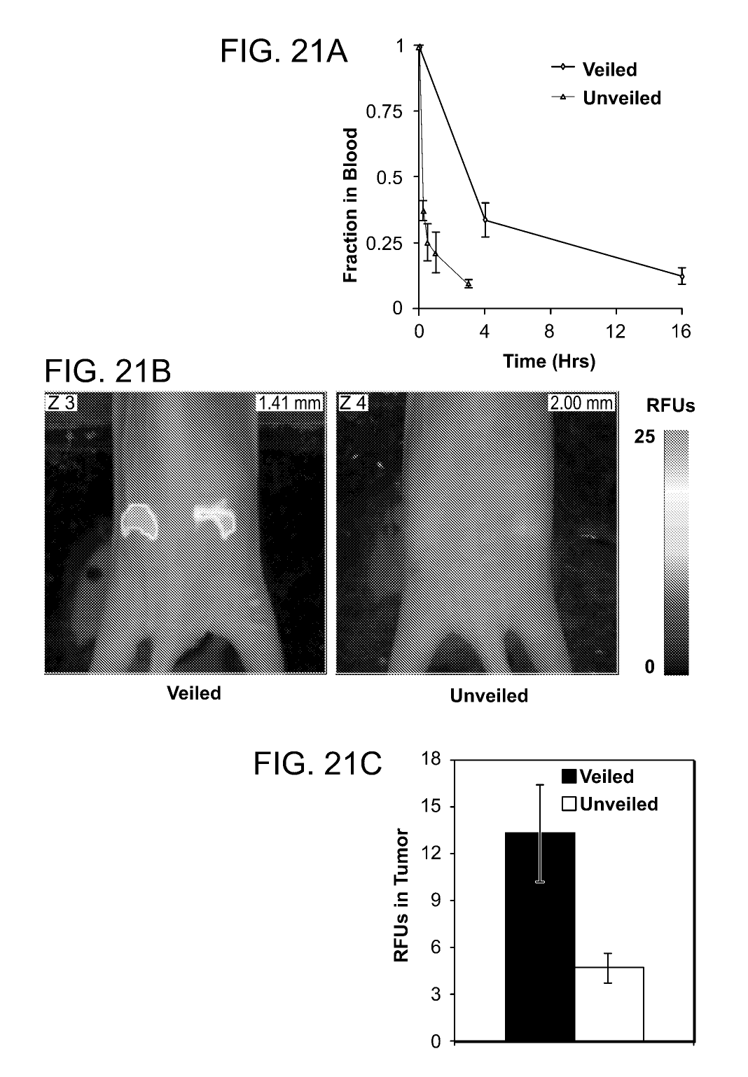

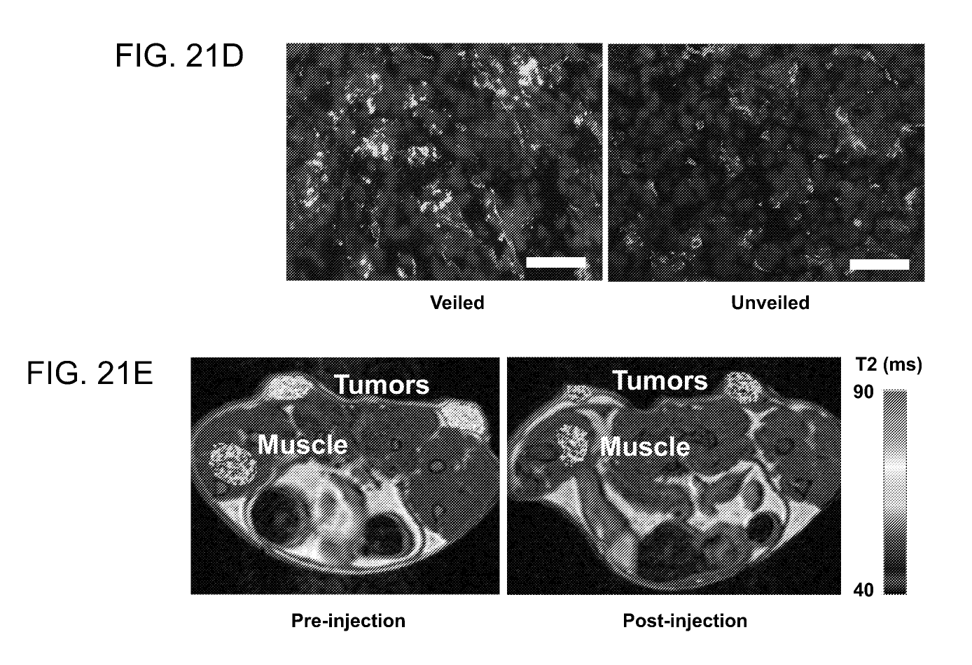

After completing these proof-of-principle experiments in vitro, we sought to investigate whether the different surface properties of veiled and unveiled particles would modify blood circulation times in vivo by systemically administering (via intravenous tail injections) veiled and unveiled nanoparticles in mice. MMP-cleaved particles had significantly lower half-lives and are cleared from the blood approximately 8 times faster than veiled particles, with more than 25% of PEG-shielded nanoparticles still in the blood at 4 hours compared to unveiled particles that had 25% remaining after only 30 minutes (FIG. 21A). The advantage of this improvement in circulation time is clearly demonstrated by the 3-fold increase in passive accumulation of veiled particles over unveiled particles in tumors as measured by fluorescence molecular tomography (FMT, FIGS. 21B,C). After 48 hours much of the injected dose has cleared from the blood so that fluorescent signal in the tumor is due primarily to extravasated particles. Histological analysis of tumors confirmed increased accumulation of veiled particles as compared to unveiled particles and shows that particles have moved beyond vascular borders (FIG. 21D). Ultimately, these results translated to post-injection knockdown of T2 relaxation times in tumors, but not normal muscle, by veiled nanoparticles administered in xenograft mice (FIG. 21E).

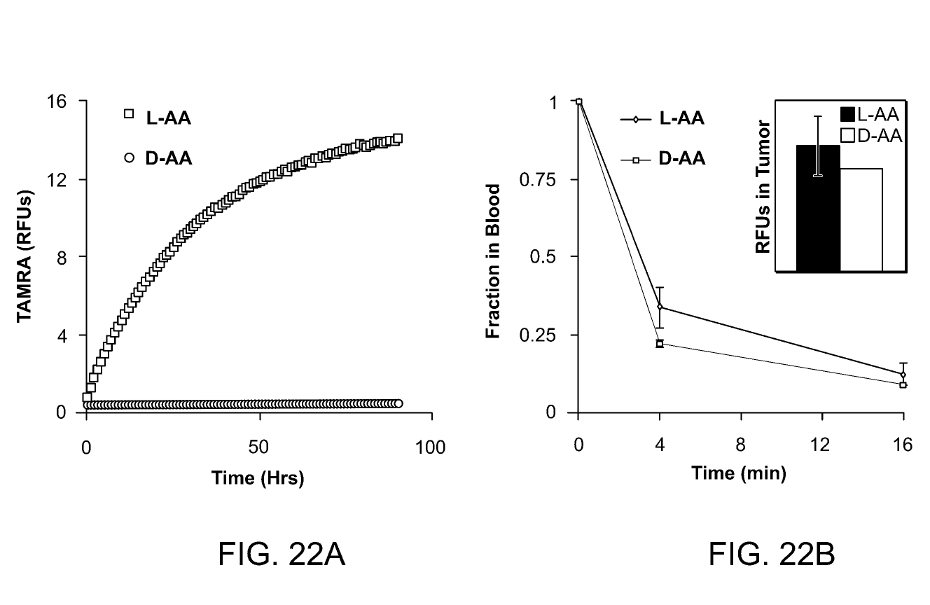

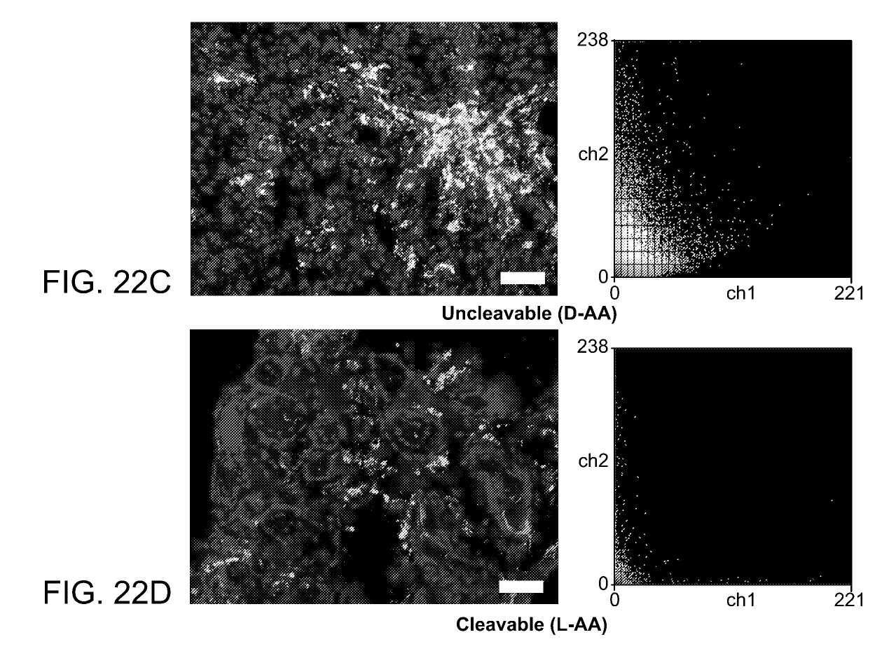

While these results generally exhibit the ability of veiled nanoparticles to passively accumulate in tumors and enable both fluorescent and magnetic-resonance tumoral imaging, we also aimed to show activation of particles by endogenous MMP expression in tumor xenografts. Toward this end, two populations of particles were synthesized and injected intravenously into xenograft mice: one cleavable with an L-AA peptide linker, and one uncleavable with a D-AA linker (FIG. 22A). The circulation times of the cleavable and uncleavable particles are closely matched, suggesting that cleavable PEG remains intact on the particle in the blood (FIG. 22B). Whole-body FMT imaging revealed similar levels of accumulation of cleavable and uncleavable probes in tumor xenografts after 48 hours, further indicating that the cleavable particle retained a biodistribution profile similar to the uncleavable version prior to exposure to the extracellular milieu of the tumor (FIG. 22B). To investigate the removal of peptide-PEG from cleavable particles by proteases in the tumor, we performed colocalization analysis on fluorescent micrographs of peptide-PEG (TAMRA-labeled) and nanoparticles (VT-680-labeled) from histological sections of tumors harvested 48 hours after injection. As expected, the fluorescence signal from the uncleavable nanoparticle was highly correlated with signal from peptide-PEG with an average Mander's Coefficient of 0.6 and a standard deviation of 0.22. The cleavable particle was significantly less correlated with an average Mander's Coefficient of 0.11 and a standard deviation of 0.13, implying that the polymer coating had been cleaved from these particles in the tumor (FIG. 22C).

The removal of the polymer in the tumor highlights a key-enabling feature of this system, which allows bioactive domains to be revealed that have been veiled in the vascular space. In this paper we have built on previous work in which the entropic penalty of PEG coatings was used to veil and unveil ligands mediating particle-particle interactions by extending this strategy to veil and unveil particle-cell interactions. Additionally we have shown that removable polymer coatings provide favorable tumor targeting properties in vivo. In the future, the incorporation of core particles carrying drug cargo or bioactive domains mediating cell-binding or signaling in this strategy could enable a functional read-out of protease-initiated unveiling in the tumor and ultimately lead to improved therapy.

### Example 14

### Coating Particles Helps Increase Particle Stability, Half-Life, and Circulation Times

C32, a poly-β amino ester constructed from bioconjugation of amino and acrylate monomers, is a vector used for gene transfer with advantages such as biodegradability and low toxicity (Anderson et al., 2004, Proc. Natl. Acad. Sci., USA, 101: 16028; incorporated herein by reference). However, for potent therapeutic efficacy, stability at physiological pH for an appreciable amount of time is typically desirable for systemic circulation and subsequent targeting of malignant sites in vivo. In an in vitro model of physiological conditions, C32-DNA nanoparticles were found to have a stable half-life of approximately 30 minutes with total particle degradation at 3 hours as shown by transfection efficiency of pGFP into MDA-435 tumor cells. With a relatively short half-life at physiological pH, applications of this gene vector are limited as prolonged circulation times are required for effective in vivo delivery to neoplastic sites. Therefore, use of polymers such as C32 to generate drug delivery particles in accordance did not appear to be promising.

However, the present invention encompasses the recognition that cloaking a polymeric particle (e.g. C32-containing particle) might extend its half-life and increase circulation times. The present invention encompasses the recognition that cloaking might increase the effectiveness of drug delivery nanoparticles comprising polymers such as C32. In particular, the present invention encompasses the unexpected result that protection from hydrolytic degradation can be accomplished using a hydrophilic polymer, such as polyethylene glycol (PEG). Therefore, an anionic, protease cleavable peptide was devised to electrostatically coat the characteristically cationic surface of C32-DNA nanoparticles. Including an anionic poly-glutamic acid and a MMP-2 substrate domain, this peptide was further functionalized by the bioconjugation of a 10 kDa polyethylene glycol tail to the MMP-2 substrate. When allowed to complex with C32-DNA nanoparticles, stabilization of the nanoplex is observed under physiological conditions at 3 hours. In addition, transfection efficiency is preserved, as demonstrated by the cleavage of the L amino acid substrate MMP-2 substrate and PEG domain, while the uncleavable D amino acid substrate particles remained at low transfection efficiency. Increasing transfection efficiency is noted with increasing L amino acid peptide-PEG coating ratios. While not wishing to be bound by any one theory, this may be due to increased steric hindrance and reduced protease degradation afforded by a more complete coverage of the C32-DNA nanoparticle surface from degradative enzymes and hydrolysis before enzymatic activation and subsequent cell transfection.

### Materials and Methods

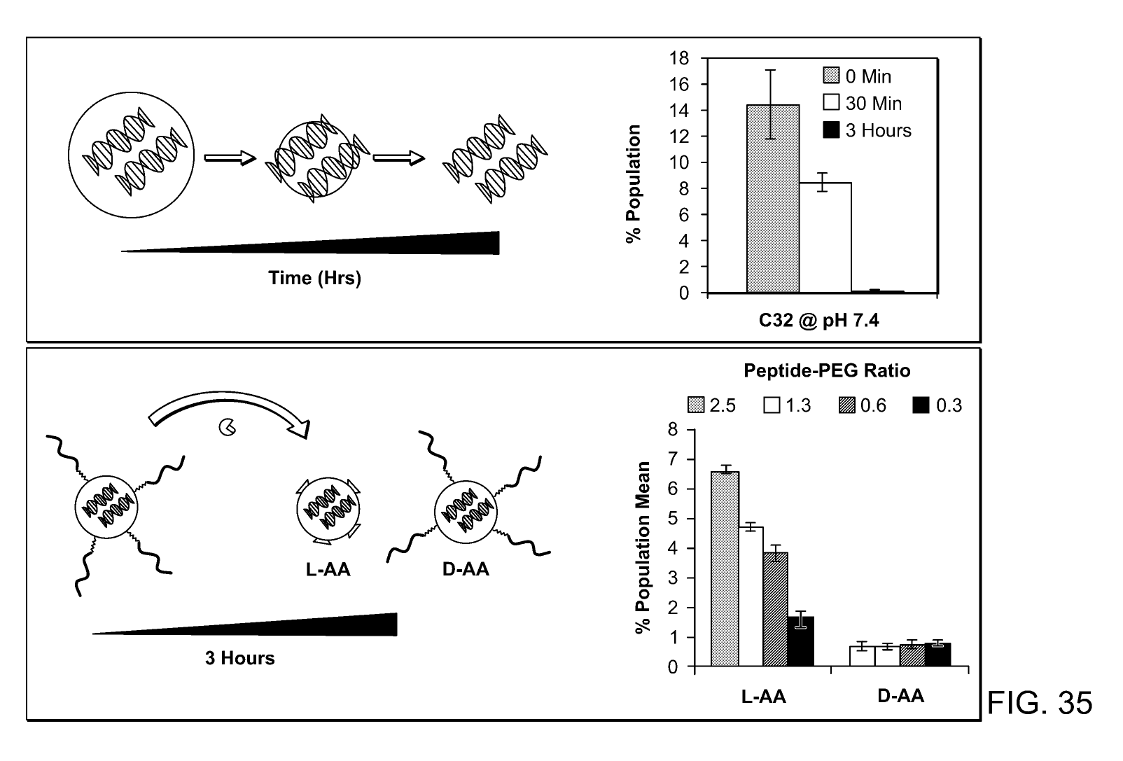

FIG. 35 (top panel): 1 mg/ml GFP DNA was diluted into 25 mM NaAC (pH) to make 0.038 mg/mL DNA solution. 100 mg/mL C32 polymer was diluted into 25 mM NaAC (pH) to make 1.52 mg/mL C32 solution. Equal volumes of DNA and C32 solutions were mixed and vortexed for 10 seconds, and allowed to incubate for 10 minutes.

In this experiment, the time after 10 minutes is referred to as “0 hours,” at which point the DNA-C32 complex solution was divided into three parts: 0 hours, 0.5 hours, and 3 hours. For each timepoint, after incubation is complete, 10×HEPES salt and 1 N NaOH were used to adjust the pH of the solution to pH 7.2. Immediately afterward, the solution to FIB with serum was mixed in a 1:5 volume ratio, vortexed for 10 seconds, and put over a clear half-96-well plate which had MDA-435 tumor cells at 70% confluency. After 72 hours, fluorescence-activated cell-sorting (FACS) was used to detect the average GFP levels of each well. Results are presented in FIG. 35.

As shown in the bottom panel of FIG. 35 (bottom panel), there were two types of 10 kD pep-PEGs used in this experiment: dAA and 1AA; all the procedures below apply to both. Using one-half gradient dilution, 10 kD pep-PEGs in 25 mM NaAC (pH 5) were made at four concentrations: 0.0475 mg/ml (2.5×), 0.0247 mg/mL (1.3×), 0.0114 mg/mL (0.6×), 0.0057 mg/mL (0.3×). After DNA and C32 conjugated for 10 minutes, the mixture was divided into four equal parts. Each 10 kD pep-PEGs solution was combined with an equal amount of C32-DNA. Each C32-DNA-PEG solution was vortexed for 10 seconds and allowed to incubate for 10 minutes. For each part, after the conjugating time is up, 10×HEPES salt and 1 N NaOH were used to bring the pH of the solutions up to pH 7.2. At this time, a small amount of collagenase solution was added into each C32-DNA-PEG sample so that the final collagenase concentration was 80 μg/ml in each sample. Immediately afterward, the solution was mixed with FIB with serum in a 1:5 volume ratio, vortexed for 10 seconds, then put over a clear half-96-well plate which had MDA-435 tumor cells at 70% confluency. Transfecting solutions were incubated with the MDA cells at 37° C. After 72 hours, FACS was used to detect the average GFP levels of each well.

### EQUIVALENTS

Those skilled in the art will recognize, or be able to ascertain using no more than routine experimentation, many equivalents to the specific embodiments, described herein. The scope of the present invention is not intended to be limited to the above Description, but rather is as set forth in the appended claims.

Those skilled in the art will recognize, or be able to ascertain using no more than routine experimentation, many equivalents to the specific embodiments in accordance with the invention described herein. The scope of the present invention is not intended to be limited to the above Description, but rather is as set forth in the appended claims.

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process. Furthermore, it is to be understood that the invention encompasses all variations, combinations, and permutations in which one or more limitations, elements, clauses, descriptive terms, etc., from one or more of the listed claims is introduced into another claim. For example, any claim that is dependent on another claim can be modified to include one or more limitations found in any other claim that is dependent on the same base claim. Furthermore, where the claims recite a composition, it is to be understood that methods of using the composition for any of the purposes disclosed herein are included, and methods of making the composition according to any of the methods of making disclosed herein or other methods known in the art are included, unless otherwise indicated or unless it would be evident to one of ordinary skill in the art that a contradiction or inconsistency would arise.

Where elements are presented as lists, e.g. in Markush group format, it is to be understood that each subgroup of the elements is also disclosed, and any element(s) can be removed from the group. It should it be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not been specifically set forth in haec verba herein. It is also noted that the term “comprising” is intended to be open and permits the inclusion of additional elements or steps.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

In addition, it is to be understood that any particular embodiment of the present invention that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the invention (e.g., any nanoparticle type, property, or material composition; any agent to be delivered; any modulating entity; any protective entity; any method of production; any method of use; etc.) can be excluded from any one or more claims, for any reason, whether or not related to the existence of prior art.

1. A conjugate comprising:
a nanoparticle;
at least one modulating entity; and
at least one agent to be delivered.

2. The conjugate of claim 1, wherein the nanoparticle comprises a polymeric matrix.

3-5. (canceled)

6. The conjugate of claim 1, wherein the nanoparticle is a non-polymeric particle.

7-9. (canceled)

10. The conjugate of claim 1, wherein the modulating entity is a targeting entity.

11. The conjugate of claim 10, wherein the targeting entity is selected from the group consisting of peptides, nucleic acids, small molecules, carbohydrates, and lipids.

12-20. (canceled)

21. The conjugate of claim 1, wherein the modulating entity is polyethylene glycol (PEG).

22. (canceled)

23. The conjugate of claim 1, wherein the agent to be delivered is a nucleic acid.

24-27. (canceled)

28. The conjugate of claim 1, wherein the agent to be delivered is a protein.

29-30. (canceled)

31. The conjugate of claim 1, wherein the agent to be delivered is a small molecule.

32-35. (canceled)

36. The conjugate of claim 1, wherein the agent to be delivered is useful for treating liver diseases.

37-46. (canceled)

47. The conjugate of claim 1, wherein the nanoparticle is covalently associated with the modulating entity.

48. (canceled)

49. The conjugate of claim 47, wherein the covalent association is mediated by a linker.

50. (canceled)

51. The conjugate of claim 49, wherein the linker is a protease-cleavable linker.

52. The conjugate of claim 51, wherein the protease-cleavable linker is cleaved by a protease, wherein the protease is preferentially expressed in tumor cells relative to non-tumor cells.

53-66. (canceled)

67. A conjugate comprising:
a nanoparticle;
a modulating entity;
polyethylene glycol (PEG); and
an agent to be delivered.

68-72. (canceled)

73. A method comprising steps of:
providing a subject suffering from, susceptible to, or displaying one or more symptoms of a disease, disorder, or condition; and
administering the conjugate of claim 1 to the subject such that the disease, disorder, or condition is treated.

74. The method of claim 73, wherein the disease, disorder, or condition is cancer.

75. The method of claim 73, wherein the disease, disorder, or condition is a liver disease.

76-86. (canceled)

87. A method comprising steps of:
providing:
a nanoparticle;
at least one modulating entity; and
at least one agent to be delivered; and
contacting the nanoparticle, at least one modulating entity, and at least one agent to be delivered such that the nanoparticle, at least one modulating entity, and at least one agent become physically associated with one another.

88. A method comprising steps of:
providing:
a nanoparticle;
at least one modulating entity; and
at least one agent to be delivered; and
contacting the nanoparticle and the at least one modulating entity to allow formation of a complex comprising the nanoparticle and the modulating entity; and
contacting the complex with the agent to be delivered.


SEQUENCE LISTING (10 sequences)

SEQ ID NO: 1 - protein, 9 aa, Artificial Sequence
  Heparan sulfate proteoglycans
CARSKNKDC

SEQ ID NO: 2 - protein, 9 aa, Artificial Sequence
  TAT peptide
RKKRRQRRR

SEQ ID NO: 3 - protein, 16 aa, Artificial Sequence
  TAT peptide
RQIKIQFZQRRMKWKK

SEQ ID NO: 4 - protein, 6 aa, Artificial Sequence
  Peptide sequences cleavable by proteases
HSSKLQ

SEQ ID NO: 5 - protein, 5 aa, Artificial Sequence
  Peptide sequences cleavable by proteases
PICFF

SEQ ID NO: 6 - protein, 7 aa, Artificial Sequence
  Peptide sequences cleavable by proteases
GPLGVRG

SEQ ID NO: 7 - protein, 10 aa, Artificial Sequence
  Peptide sequences cleavable by proteases
GVSQNYPIVG

SEQ ID NO: 8 - protein, 10 aa, Artificial Sequence
  Peptide sequences cleavable by proteases
LVLASSSFGY

SEQ ID NO: 9 - protein, 4 aa, Artificial Sequence
  Peptide sequences cleavable by proteases
DEVD

SEQ ID NO: 10 - protein, 4 aa, Artificial Sequence
  Peptide sequences cleavable by proteases
WEHD